(19) United States
(12) Patent Application Publication
(10) Pub. No.: US 2023/0089828 A1
(43) Pub. Date: Mar. 23, 2023

(54) USE OF EARLY APOPTOTIC CELLS FOR TREATING COVID-19

(71) Applicant: ENLIVEX THERAPEUTICS R&D LTD, Nes-Ziona (IL)

(72) Inventors: Shai NOVIK, Ramat Hasharon (IL); Dror MEVORACH, Jerusalem (IL)

(73) Assignee: ENLIVEX THERAPEUTICS R&D LTD, Nes-Ziona (IL)

(21) Appl. No.: 17/904,223

(22) PCT Filed: Feb. 21, 2021
(86) PCT No.: PCT/IL2021/050198

§ 371 Date: Aug. 15, 2022

Related U.S. Application Data
(60) Provisional application No. 62/980,457, filed on Feb. 24, 2020.
(60) Provisional application No. 63/085,711, filed on Sep. 30, 2020.
(60) Provisional application No. 63/086,079, filed on Oct. 1, 2020.
(60) Provisional application No. 63/145,524, filed on Feb. 4, 2021.

(51) Int. Cl. A61K 35/17; A61P 31/14; A61K 9/00

(52) U.S. Cl. CPC A61K 35/17; A61P 31/14; A61K 9/0019

(57) ABSTRACT

Compositions disclosed herein, and methods of use thereof included those for treating or preventing SARS-CoV-2 vims infection in a subject in need, including methods of extending of the survival of a subject suffering from COVID-19, and reduction of organ dysfunction or failure due to COVID-19 or associated symptoms. Methods of treating or preventing COVID-19 in a subject in need includes administering compositions comprising early apoptotic cells or early apoptotic cell supernatants. Compositions and methods of use thereof may reduce the negative proinflammatory effect accompanying COVID-19 and symptoms thereof. Further, anti-inflammatory cytokine release may be increased. In certain instances, compositions may include additional agents.

### FIELD OF INTEREST

Disclosed herein are compositions comprising early apoptotic cells or a supernatant thereof, for treating COVID-19. The use treating COVID-19 may inhibit or reduce the incidence of cytokine release syndrome (CRS) or a cytokine storm in a subject suffering from COVID19. Compositions disclosed herein may be used for treating the symptoms of a SARS-CoV-2 infection in a subject, and for increasing survival of a COVID-19 subject. Compositions used may be administered alone or in combination with other therapies.

### BACKGROUND

COVID-19, the name given to the clinical syndrome associated with the newly recognized virus SARS-CoV-2, has become pandemic with mortality estimated at between 1-3% (based on reports from China) and complications among hospitalized patients leading to up to 15-25% admissions to intensive care units. The clinical presentation of COVID-19 includes both upper and lower respiratory tract infection, but patients may also be asymptomatic. A diagnostic PCR assay was rapidly developed in Hong Kong and Berlin that accurately detects SARS-CoV-2 in samples from nose and throat swabs or sputum of hospitalized patients, and is used by public-health authorities around the world. To avoid cross-reactivity with SARS-CoV or other coronaviruses, the test detects a region of the gene encoding RNA-dependent RNA polymerase that is unique to SARS-CoV-2. Not all patients need hospitalization but due to high index of infection spread, all detected patients are put in isolation to prevent transmission of the infection to others.

The development of vaccines is undoubtedly an important step and several MERS vaccines were already in clinical trials when word of the new outbreak spread. However, developing and testing the correct viral protein that will be effective (and probably not 100% protective) will take some time. In the meantime, anti-viral agents are being tested. These include a combination of two human immunodeficiency virus (HIV) antivirals. Lopinavir and ritonavir have been taking center stage as potential therapies for COVID-19, and there are at least three registered randomized clinical trial testing the lopinavir-ritonavir combination in Chinese patients infected with SARS-CoV-2 (NCT04255017, NCT04252885 and NCT04251871), with the results of one being negative, as published in the New England of Medicine in March 2020. A handful of other HIV antivirals are currently in clinical testing against SARS-CoV-2, including darunavir-cobicistat, which was donated by the US pharmaceutical company Johnson & Johnson to the Shanghai Public Health Clinical Center.

Nucleoside analogues are being considered too and remdesivir was used to treat the first US patient infected with SARS-CoV-2, who recovered. It is also in phase 3 trials in Wuhan patients infected with SARS-CoV-2, overseen by the China-Japan Friendship Hospital in Beijing (NCT04252664 and NCT04257656). However, SARS-CoV-2 virus has its own proteases, including Corona main protease, M-pro, and the HIV antivirals are designed and tailored to specifically to block the activity of HIV proteases in order to avoid off-target effects on human cells. This makes HIV antivirals less likely to bind SARS-CoV-2 proteases as well. Chloroquine was recently suggested as an additional anti-viral medication. In addition, even if anti-viral therapy will be found to be efficacious against SARS-CoV-2, it is unclear if this will be the treatment of choice in patients admitted to the ICU.

The term “cytokine storm” calls up vivid images of an immune system gone awry and an inflammatory response flaring out of control. The term has captured the attention of the public and the scientific community alike and is increasingly being used in both the popular media and the scientific literature. Indeed, a few publications have indicated an important part of the complications in COVID19 are related to a cytokine storm (Huang et al. (2020) Lancet vol. 395:497-506; Mehta et al. (2020) Lancet vol. 395:1033-1034).

Cytokine release syndrome (CRS) is a dangerous and sometimes life-threatening side effect, in which cells produce a systemic inflammatory response in which there is a rapid and massive release of cytokines into the bloodstream, leading to dangerously low blood pressure, high fever and shivering.

In severe cases of CRS, patients experience a cytokine storm (a.k.a. cytokine cascade or hypercytokinemia), in which there is a positive feedback loop between cytokines and white blood cells with highly elevated levels of cytokines. This can lead to potentially life-threatening complications including cardiac dysfunction, adult respiratory distress syndrome, neurologic toxicity, renal and/or hepatic failure, pulmonary edema and disseminated intravascular coagulation.

For example, six patients in a recent phase I trial who were administered the monoclonal antibody TGN1412, which binds to the CD28 receptor on T-cells, exhibited severe cases of cytokine storm and multi-organ failure. This happened despite the fact that the TGN1412 dose was 500-times lower than that found to be safe in animals (St. Clair E W: The calm after the cytokine storm: Lessons from the TGN1412 trial. J Clin Invest 118: 1344-1347, 2008).

Cytokine storms are also a problem after other infectious and non-infectious stimuli. In a cytokine storm, numerous proinflammatory cytokines, such as interleukin-1 (IL-1), IL-6, g-interferon (g-IFN), and tumor necrosis factor-α (TNFα), are released, resulting in hypotension, hemorrhage, and, ultimately, multiorgan failure. The relatively high death rate in young people, with presumably healthy immune systems, in the 1918 H1N1 influenza pandemic and the more recent bird flu H5N1 infection are attributed to cytokine storms. This syndrome has been also known to occur in advanced or terminal cases of severe acute respiratory syndrome (SARS), Epstein-Barr virus-associated hemophagocytic lymphohistiocytosis, sepsis, gram-negative sepsis, malaria and numerous other infectious diseases, including Ebola infection.

The immune system usually works to fight any germs (bacteria, viruses, fungi or parasites) to prevent infection. If an infection does occur, the immune system will try to fight it, although it may need help from medication such as antibiotics, antivirals, antifungals and antiparasitics.

Apoptotic cells present one pathway of physiological cell death, most commonly occurring via apoptosis, which elicits a series of molecular homeostatic mechanisms comprising recognition, an immune response and a removal process. Moreover, early apoptotic cells are immunomodulatory cells capable of directly and indirectly inducing immune tolerance to dendritic cells and macrophages. Apoptotic cells have been shown to modulate dendritic cells and macrophages and to render them tolerogenic and inhibit proinflammatory activities such as secretion of proinflammatory cytokines and expression of costimulatory molecules.

As many as 3×10⁸ cells undergo apoptosis every hour in the human body. One of the primary “eat me” signals expressed by apoptotic cells is phosphatidylserine (PtdSer) membrane exposure. Apoptotic cells themselves are major contributors to the “non-inflammatory” nature of the engulfment process, some by secreting thrombospondin-1 (TSP-1) or adenosine monophosphate and possibly other immune modulating “calm-down” signals that interact with macrophages and DCs. Apoptotic cells also produce “find me” and “tolerate me” signals to attract and immunomodulate macrophages and DCs that express specific receptors for some of these signals.

The pro-homeostatic nature of apoptotic cell interaction with the immune system is illustrated in known apoptotic cell signaling events in macrophages and DCs that are related to Toll-like receptors (TLRs), NF-κB, inflammasome, lipid-activated nuclear receptors, Tyro3, Axl, and Mertk receptors. In addition, induction of signal transducers, activation of transcription 1, and suppression of cytokine signaling lead to immune system silencing and DC tolerance (Trahtemberg, U., and Mevorach, D. (2017). Apoptotic cells induced signaling for immune homeostasis in macrophages and dendritic cells. Front. Immunol. 8; article 1356).

As summarized recently (Trahtemberg and Mevorach, 2017, ibid), early apoptotic cells may have a beneficial effect on aberrant immune response, with downregulation of both anti- and pro-inflammatory cytokines derived from PAMPs and DAMPs, in both animal and in vitro models. In that regard a phase 1b clinical trial of immune modulation in patients with sepsis was recently completed, with the main results being that Allocetra-OTS, an early apoptotic cell infusion proved to be safe and had a significant immuno-modulating effect, leading to resolution of the cytokine storm.

Interestingly, in a recent study by Zou et al (2020) Lancet vol. 395:1054-1062, of 191 patients (135 from Jinyintan Hospital and 56 from Wuhan Pulmonary Hospital) with COVID-19 of whom 137 were discharged and 54 died in hospital. 91 (48%) patients had a comorbidity, with hypertension being the most common (58 [30%] patients), followed by diabetes (36 [19%] patients) and coronary heart disease (15 [8%] patients). Multivariable regression showed increasing odds of in-hospital death in these COVID-19 patients associated with older age and higher Sequential Organ Failure Assessment (SOFA) score on admission. The authors pointed out the potential risk factors besides older age, are high SOFA score and d-dimer greater than 1 μg/ml.

Symptoms observed in moderate to severe COVID19 patients may include a comparable underlying immunological mechanism of action similar to the one that was recently shown in sepsis, but that knowledge is unknown. Forty previous trials using monoclonal antibodies against a single cytokine in septic patients have failed (Cohen et al 2012) in sepsis pointing out that there is a need to modify the cytokine storm rather than treating with a single anti-cytokine.

There remains an unmet need for compositions and methods of treatment of COVID-19 and associated symptoms, such as respiratory infections and multi-organ failure, in subjects infected with the SARS-CoV-2 virus.

The methods of use described herein including the use of early apoptotic mononuclear-enriched cells, which address this need and address increasing the survival time of a subject suffering from SARS-CoV-2 virus infection (COVID-19).

### SUMMARY

In one aspect disclosed herein is a method of treating COVID-19 in a subject infected by SARS-CoV-2 virus, said method comprising administering a composition comprising an early apoptotic mononuclear-cell-enriched population to the subject, wherein said administration treats COVID-19. In a related aspect, treating comprises treating, inhibiting, reducing the incidence of, ameliorating, or alleviating a symptom of COVID-19.

In a further related aspect, a COVID-19 symptom comprises organ failure, organ dysfunction, organ damage, a cytokine storm, a cytokine release syndrome, or a combination thereof. In yet another further related aspect, an organ comprises a lung, a heart, a kidney, or a liver, or any combination thereof. In still another further related aspect, organ dysfunction, failure, or damage comprises lung dysfunction, failure, or damage. In certain aspects, lung dysfunction comprises acute respiratory distress syndrome (ARDS) or pneumonia. In a further related aspect, organ failure comprises acute multiple organ failure. In a related aspect, treating organ failure comprises reducing, slowing, inhibiting, reversing, or repairing said organ failure, or a combination thereof.

In a related aspect, a method of treating COVID-19 increases survival time of a COVID-19 subject, compared with a COVID-19 subject not administered said early apoptotic mononuclear-cell-enriched population. In a further related aspect, the COVID-19 comprises moderate or severe COVID-19. In a further related aspect, the COVID-19 comprises moderate, severe, or critical COVID-19. In a further related aspect, the COVID-19 comprises severe or critical COVID-19. In a further related aspect, the COVID-19 comprises severe COVID-19. In a further related aspect, the COVID-19 comprises critical COVID-19.

In a related aspect, early apoptotic mononuclear-cell-enriched populations used in methods of treating COVID-19 comprises (a) an apoptotic population stable for greater than 24 hours; (b) a decreased number of non-quiescent non-apoptotic cells, a suppressed cellular activation of any living non-apoptotic cells, or a reduced proliferation of any living non-apoptotic cells, or (c) a pooled population of early apoptotic mononuclear-enriched cells, or (d) any combination thereof.

In a related aspect for methods of treating COVID-19, administering comprises a single infusion of the early apoptotic mononuclear-cell-enriched population. In a further related aspect, administering comprises multiple infusions of the early apoptotic mononuclear-cell-enriched population. In yet a further related aspect, administering comprises intravenous (IV) administration.

In a related aspect for methods of treating COVID-19, an early apoptotic mononuclear-cell-enriched population comprises early apoptotic cells irradiated after induction of apoptosis.

In a related aspect, a method of treating COVID-19 includes a step of administering an additional therapy in addition to administration of early apoptotic cells. In a further related aspect, the additional therapy is administered prior to, concurrent with, or following the step of administering the early apoptotic mononuclear-cell-enriched population.

In a related aspect, a method of treating COVID-19 comprises rebalancing the immune response of the subject. In a further related aspect, rebalancing comprises reducing the secretion of one or more proinflammatory cytokines, anti-inflammatory cytokines, chemokine, or immune modulator, or a combination thereof. In yet a further related aspect, rebalancing comprises increasing the secretion of one or more anti-inflammatory cytokine or chemokine, or combination thereof. In still a further related aspect, rebalancing comprises reducing secretion of one or more pro- or anti-inflammatory cytokine or chemokine or immune modulator, and increasing one or more anti-inflammatory cytokine or chemokine.

In a related aspect, treatment of COVID-19 with early apoptotic mononuclear-enriched cells reduces the subject's stay in an intensive care unit (ICU), compared with a subject not administered early apoptotic mononuclear-enriched cells. In another related aspect, treatment of COVID-19 with early apoptotic mononuclear-enriched cells reduces hospitalization time for said subject, compared with a subject not administered early apoptotic mononuclear-enriched cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter disclosed herein is particularly pointed out and distinctly claimed in the concluding portion of the specification. The compositions and methods disclosed herein, however, both as to organization and method of operation, together with objects, features, and advantages thereof, may best be understood by reference to the following detailed description when read with the accompanying drawings.

FIG. 1 presents a flow chart of the steps presenting some embodiments of a manufacturing process of early apoptotic cell populations, wherein anti-coagulants were included in the process (See the Examples, e.g., Example 1). The peripheral blood mononuclear cells (PBMC) collected may in some embodiments be autologous and in other embodiments be allogeneic, wherein non-matched mononuclear cells may be used in some embodiments. Additional step includes irradiating the cells following induction of apoptosis and pooling unmatched cells if multiple sources of cells were used.

FIGS. 2A-2B. Potency Test. FIGS. 2-2B present the results of a potency test that shows the inhibition of maturation of dendritic cells (DCs) following interaction with apoptotic cells, measured by expression of HLA-DR. FIG. 2A. HLA DR mean fluorescence of fresh final product A (t0). FIG. 2B. HLA DR mean fluorescence of final product A, following 24 h at 2-8° C.

FIGS. 3A-3B. Potency Test. FIGS. 3A-3B present the results of a potency test that shows the inhibition of maturation of dendritic cells (DCs) following interaction with apoptotic cells, measured by expression of CD86. FIG. 4A. CD86 Mean fluorescence of fresh final product A (t0). FIG. 3B. CD86 Mean fluorescence of final product A, following 24 h at 2-8° C.

FIG. 4. Schematic of Phase II COVID-19 Trial for Safety and Evaluation of Efficacy.

FIGS. 5A-5L. Phase I COVID-19 positive biomarkers' profile over time (per day). Markers included WBC (FIG. 5A), Neutrophil % (FIG. 5B), Neutrophil Count (FIG. 5C), Lymphocyte % (FIG. 5D), Lymphocyte count (FIG. 5E), Platelet Count (FIG. 5F), CRP (FIG. 5G), Ferritin (FIG. 5H), D-dimer (FIG. 5I), CPK (FIG. 5J), Creatinine (FIG. 5K), and LDH (FIG. 5L). Red circle indicates healthy controls, and patients are represented by the square (patient 1), upward triangle (patient 2), downward triangle (patient 3), diamond (patient 5), and open circle (patient 6).

FIGS. 6A-6H. Phase I COVID-19 positive cytokine profile over time (per day). Cytokines measured included IL-6 (FIG. 6A), IL-18 (FIG. 6B), IFN-α (FIG. 6C), IFN-γ (FIG. 6D), IL-10 (FIG. 6E), IL-2Ra (FIG. 6F), IL-8 (FIG. 6G), and IL-7 (FIG. 6H). Red circle indicates healthy controls, and patients are represented by the square (patient 1), upward triangle (patient 2), downward triangle (patient 3), diamond (patient 5), and open circle (patient 6). Red circle indicates healthy controls, and patients are represented by the square (patient 1), upward triangle (patient 2), downward triangle (patient 3), diamond (patient 5), and open circle (patient 6).

FIGS. 7A-7O show the interim measurements of Phase II COVID-19 positive biomarkers' profile over time (per day). Markers included CRP (FIG. 7A), Ferritin (FIG. 7B), D-dimer (FIG. 7C), CPK (FIG. 7D), Creatinine (FIG. 7E), WBC (FIG. 7F), Neutrophil % (FIG. 7G), Neutrophil Count (FIG. 7H), Lymphocyte % (FIG. 7I), Lymphocyte Count (FIG. 7J), Aspartate transaminase (AST) (FIG. 7K), Alanine aminotransferase (ALT) (FIG. 7L), Alkaline phosphatase (ALP) (FIG. 7M), Total Bilirubin (FIG. 7N), and Lactate dehydrogenase (LDH) (FIG. 7O). Red circle indicates reference range, and patients are represented by the square (patient 01-001), upward triangle (patient 01-002), downward triangle (patient 01-003), diamond (patient 01-004), open circle (patient 01-005), open square (patient 01-007), open upward triangle (patient 01-008), and open downward triangle (patient 01-009).

### DETAILED DESCRIPTION

In the following detailed description, numerous specific details are set forth in order to provide a thorough understanding of the methods disclosed herein. However, it will be understood by those skilled in the art that these methods may be practiced without these specific details. In other instances, well-known methods, procedures, and components have not been described in detail so as not to obscure the methods disclosed herein.

The clinical presentation of COVID-19 starts with an estimated incubation period of up to 14 days from the time of exposure. The spectrum of illness can range from asymptomatic infection to severe pneumonia with acute respiratory distress syndrome (ARDS) and death. Severe cases of COVID-19 may be associated with acute respiratory distress syndrome and elevations in multiple inflammatory cytokines that provoke a cytokine storm, and/or exacerbation of underlying comorbidities. Among persons with COVID-19 cases have been reported to be mild (no pneumonia or mild pneumonia), severe (defined as dyspnea, respiratory frequency ≥30 breaths/min, SpO₂≤93%, PaO₂/FiO₂<300 mmHg, and/or lung infiltrates >50% within 24 to 48 hours), and critical (defined as respiratory failure, septic shock, and/or multiple organ dysfunction or failure). In addition to pulmonary disease, patients with COVID-19 may also experience cardiac, hepatic, renal, and central nervous system disease.

COVID-19 is the name of the new disease found in a subject infected with a severe acute respiratory syndrome coronavirus 2 (SARS-CoV-2). In some embodiments, the terms “COVID-19”, “COVID19”, “COVID”, and “Corona” may be used interchangeably having all the same qualities and meanings in reference to the disease in a human subject as the result of a SARS-CoV-2 infection. In some embodiments, the terms “SARS-CoV-2”, “Corona virus”, and “Corona” may be used interchangeably having all the same qualities and meanings in reference to the virus that causes the COVID-19 disease. One skilled in the art would appreciate from the context whether reference is being made to the disease or the virus.

According to the NIH Guidelines, “patients with COVID-19 may express high levels of an array of inflammatory cytokines, often in the setting of deteriorating hemodynamic or respiratory status. This is often referred to as “cytokine release syndrome” or “cytokine storm,” although these are imprecise terms. Intensivists need to consider the full differential diagnosis of shock to exclude other treatable causes of shock (e.g., bacterial sepsis due to pulmonary or extrapulmonary sources, hypovolemic shock due to a gastrointestinal hemorrhage that is unrelated to COVID-19, cardiac dysfunction related to COVID-19 or comorbid atherosclerotic disease, stress-related adrenal insufficiency).” (NIH: COVID-19 Treatment Guidelines; https://www.covid19treatmentguidelines.nih.gov/).

Organ failure in COVID-19, is considered the result of an exaggerated response of the immune system (“cytokine storm”) in the human body to an infection by a virus or a bacterium.

This exaggerated immune response results in organ damage. The immune attacks typically occur in vital organs such as lungs, heart, kidney, liver and others. The organs are distressed, and they begin to slowly dysfunction, and could move into organ failure, multiple organ failure, and mortality. A cytokine storm was recently reported in patients with COVID-19 that were hospitalized in the ICU (Huang et al. www.thelancet.com Published online Jan. 24, 2020 https://doi.org/10.1016/S0140-6736(20)30183-5) and patients admitted to ICU had higher plasma levels of cytokines and chemokines.

There is no approved treatment for COVID-19 at this time.

In some embodiments, disclosed herein are methods of treating COVID-19 in a subject infected by the SARS-CoV-2 virus, comprising administering a composition comprising an early apoptotic mononuclear-cell-enriched population to the subject, wherein said administration treats COVID-19. In some embodiments, treating comprises treating at least one symptom of COVID-19.

In some embodiments, disclosed herein are compositions comprising early apoptotic cells. In some embodiments, disclosed herein are compositions comprising early apoptotic cells in combination with an additional agent. In some embodiments, the additional agent comprises a therapeutic agent for treating COVID-19 and symptoms thereof.

In some embodiments, this disclosure provides methods of production of a pharmaceutical composition comprising a pooled mononuclear apoptotic cell preparation comprising pooled individual mononuclear cell populations in an early apoptotic state, wherein said composition comprises a decreased percent of living non-apoptotic cells, a preparation having a suppressed cellular activation of any living non-apoptotic cells, or a preparation having reduced proliferation of any living non-apoptotic cells, or any combination thereof. In another embodiment, the methods provide a pharmaceutical composition comprising a pooled mononuclear apoptotic cell preparation comprising pooled individual mononuclear cell populations in an early apoptotic state, wherein said composition comprises a decreased percent of non-quiescent non-apoptotic cells.

In some embodiments, disclosed herein is a method of treating COVID-19 in a subject infected by SARS-CoV-2, comprising a step of administering an early apoptotic mononuclear-enriched cell population to said subject, wherein said method treats COVID-19. In some embodiments, methods of treating herein comprise treating, inhibiting, reducing the incidence of, ameliorating, or alleviating a symptom of COVID-19.

In some embodiments, disclosed herein are methods of inhibiting or reducing the incidence of cytokine release syndrome (CRS) or cytokine storm in a subject. In another embodiment, methods disclosed herein decrease or prevent cytokine production in a subject thereby inhibiting or reducing the incidence of cytokine release syndrome (CRS) or cytokine storm in a subject. In another embodiment, the methods disclosed herein of inhibiting or reducing the incidence of cytokine release syndrome (CRS) or cytokine storm in a subject comprise the step of administering a composition comprising early apoptotic mononuclear-enriched cells to the subject. In yet another embodiment, methods disclosed herein for decreasing or inhibiting cytokine production in a subject comprise the step of administering a composition comprising early mononuclear-enriched apoptotic cells to the subject.

In some embodiments, disclosed herein are methods of decreasing or inhibiting cytokine production in a subject experiencing cytokine release syndrome or cytokine storm or vulnerable to cytokine release syndrome or cytokine storm comprising the step of administering an early apoptotic cell supernatant, as disclosed herein, or a composition comprising said apoptotic cell supernatant. In another embodiment, an early apoptotic cell supernatant comprises an apoptotic cell-phagocyte supernatant.

In some embodiments, a method of inhibiting or reducing the incidence of a cytokine release syndrome (CRS) or a cytokine storm in a subject comprises the step of administering a composition comprising early apoptotic cells or an early apoptotic supernatant to said subject. In another embodiment, a method of inhibiting or reducing the incidence of a cytokine release syndrome (CRS) or a cytokine storm in a subject, decreases or inhibits production of at least one pro-inflammatory cytokine in the subject.

In another embodiment, this disclosure provides methods of use of a pooled mononuclear early apoptotic cell preparation comprising mononuclear cells in an early apoptotic state, as described herein, for treating a symptom of COVID-19, comprising a cytokine release syndrome (CRS), a cytokine storm, reduced organ function, or organ failure, or a combination thereof in a subject in need thereof. In another embodiment, disclosed herein is a pooled mononuclear apoptotic cell preparation, wherein use of such a cell preparation in certain embodiments does not require matching donors and recipients, for example by HLA typing.

### Cytokine Storm and Cytokine Release Syndrome

In certain embodiments the cytokine release syndrome (CRS), severe CRS (sCRS), or cytokine storm occurs as a result of a SARS-CoV-2 infection. In one embodiment, a cytokine storm, cytokine cascade, or hypercytokinemia is a more severe form of cytokine release syndrome.

Various studies noted the accumulation of activated macrophages in the lungs and dysregulated activation of the mononuclear phagocyte (MNP) compartment, which contributes to COVID-19-associated hyperinflammation. These cells were shown to be elevated in bronchoalveolar fluid from patients with mild to severe COVID-19. Moreover, MNP composition was further characterized by a depletion of tissue-resident alveolar macrophages and an abundance of inflammatory monocyte-derived macrophages in patients with severe disease. These cells have a strong interferon gene signature.

Based on some recent studies, it appears that COVID-19 complications are not a classical CRS associated symptom, but the pathogenicity of infiltrating macrophages could extend beyond the promotion of acute inflammation. So, still, controlling the response of activated macrophages seems to be a key in the treatment of mild to severe or critical COVID-19 patients. In certain embodiments, administering Allocetra-OTC (early apoptotic cells) plays a role in the treatment of CRS associated symptoms of COVID-19. In some embodiments, treatment with Allocetra-OTC ameliorates activated macrophages and reduces CRS. (See for example Merad and Martin (2020) Pathological inflammation in patients with COVID-19: a key role for monocytes and macrophages. Nature Reviews Immunology, 20: 355-362, incorporated herein in its entiretyFresp.)

In some embodiment, an agent for decreasing harmful cytokine release comprises early apoptotic cells or a composition comprising said early apoptotic cells. In another embodiment, an agent for decreasing harmful cytokine release comprises an early apoptotic cell supernatant or a composition comprising said supernatant. In another embodiment, the additional agent for decreasing harmful cytokine release comprises a CTLA-4 blocking agent. In another embodiment, the additional agent for decreasing harmful cytokine release comprises apoptotic cells or apoptotic cell supernatants or compositions thereof, and a CTLA-4 blocking agent. In another embodiment, the additional agent for decreasing harmful cytokine release comprises an alpha-1 anti-trypsin or fragment thereof or analogue thereof. In another embodiment, the additional agent for decreasing harmful cytokine release comprises early apoptotic cells or early apoptotic cell supernatants or compositions thereof, and an alpha-1 anti-tryp sin or fragment thereof or analogue thereof. In another embodiment, the additional agent for decreasing harmful cytokine release comprises a tellurium-based compound. In another embodiment, the additional agent for decreasing harmful cytokine release comprises early apoptotic cells or early apoptotic cell supernatants or compositions thereof, and a tellurium-based compound. In another embodiment, the additional agent for decreasing harmful cytokine release comprises an immune modulating agent. In another embodiment, the additional agent for decreasing harmful cytokine release comprises early apoptotic cells or early apoptotic cell supernatants or compositions thereof, and an immune modulating agent. In another embodiment, the additional agent for decreasing harmful cytokine release comprises Treg cells. In another embodiment, the additional agent for decreasing harmful cytokine release comprises early apoptotic cells or early apoptotic cell supernatants or compositions thereof, and Treg cells.

A skilled artisan would appreciate that decreasing toxic cytokine release or toxic cytokine levels comprises decreasing or inhibiting production of toxic cytokine levels in a subject, or inhibiting or reducing the incidence of cytokine release syndrome or a cytokine storm in a subject. In another embodiment toxic cytokine levels are reduced during CRS or a cytokine storm. In another embodiment, decreasing or inhibiting the production of toxic cytokine levels comprises treating CRS or a cytokine storm. In another embodiment, decreasing or inhibiting the production of toxic cytokine levels comprises preventing CRS or a cytokine storm. In another embodiment, decreasing or inhibiting the production of toxic cytokine levels comprises alleviating CRS or a cytokine storm. In another embodiment, decreasing or inhibiting the production of toxic cytokine levels comprises ameliorating CRS or a cytokine storm. In another embodiment, the toxic cytokines comprise pro-inflammatory cytokines. In another embodiment, pro-inflammatory cytokines comprise IL-6. In another embodiment, pro-inflammatory cytokines comprise IL-1(3. In another embodiment, pro-inflammatory cytokines comprise TNF-α. In another embodiment, pro-inflammatory cytokines comprise IL-6, IL-1(3, or TNF-α, or any combination thereof.

In one embodiment, cytokine release syndrome is characterized by elevated levels of several inflammatory cytokines and adverse physical reactions in a subject such as low blood pressure, high fever and shivering. In another embodiment, inflammatory cytokines comprise IL-6, IL-1(3, and TNF-α. In another embodiment, CRS is characterized by elevated levels of IL-6, IL-1(3, or TNF-α, or any combination thereof. In another embodiment, CRS is characterized by elevated levels of IL-8, or IL-13, or any combination thereof. In another embodiment, a cytokine storm is characterized by increases in TNF-alpha, IFN-gamma, IL-1beta, IL-2, IL-6, IL-8, IL-10, IL-13, GM-CSF, IL-5, fracktalkine, or a combination thereof or a subset thereof. In yet another embodiment, IL-6 comprises a marker of CRS or cytokine storm.

In another embodiment, cytokines increased in CRS or a cytokine storm in humans and mice may comprise any combination of cytokines listed in Tables 1 and 2 below.

TABLE 1
Panel of Cytokines Increased in CRS or Cytokine Storm in Humans and/or Mice

| | Human | Mouse model (pre- | | | |
|---|---|---|---|---|---|
| | model | clinical) | | | |
| Cytokine | (clinical | Mouse | Not | | Notes/ |
| (Analyte) | trials) | origin | specified | Cells secreting this cytokine | other |
| Flt-3L | * | | | DC (?) | |
| Fractalkine | * | | | APC, Endothelial cells (?) | =CX3CL1, |
| | | | | | Neurotactin |
| | | | | | (Mouse) |
| M-CSF | | | | | =CSF1 |
| GM-CSF | * | | * (in vitro) | T cell, MØ | |
| IFN- | * | | | T cell, MØ, Monocyte | |
| alpha | | | | | |
| IFN-beta | ? | | ? | T cell, MØ, Monocyte | |
| IFN- | * | | * (in vitro) | cytotoxic T cells, helper T cells, | |
| gamma | | | | NK cells, MØ, Monocyte, DC | |
| IL-1 | * | | | Monocyte, MØ, Epithel | |
| alpha | | | | | |
| IL-1 beta | * | | * | Macrophages, DCs, fibroblasts, | |
| | | | | endothelial cells, hepatocytes | |
| IL-1 R | * | | | | |
| alpha | | | | | |
| IL-2 | * | | * (in vitro) | T cells | |
| IL-2R | * | | | lymphocytes | |
| alpha | | | | | |
| IL-4 | * | | * (in vitro) | Th2 cells | |
| IL-5 | * | | * | T cells | |
| IL-6 | * | * | * | monocytes/macrophages, dendritic | |
| | | | | cells, T cells, fibroblasts, | |
| | | | | keratinocytes, endothelial cells, | |
| | | | | adipocytes, myocytes, mesangial | |
| | | | | cells, and osteoblasts | |
| IL-7 | * | | * | In vitro by BM stromal cells | |
| IL-8 | * | | | Macrophages, monocytes | |
| IL-9 | * | | | T cells, T helper | |
| IL-10 | * | * | * (in vitro) | monocytes/macrophages, mast | |
| | | | | cells, B cells, regulatory T cells, | |
| | | | | and helper T cells | |
| IL-12 | * | | * | MØ, Monocyte, DC, activated | = p70 |
| | | | | lymphocytes, neutrophils | (p40 + p35) |
| IL-13 | * | | | T cells | |

In some embodiments, cytokines Flt-3L, Fractalkine, GM-CSF, IFN-γ, IL-1(3, IL-2, IL-2Rα, IL-4, IL-5, IL-6, IL-7, IL-8, IL-9, IL-10, IL-12, and IL-13 of Table 1 are considered to be significant in CRS or cytokine storm. In another embodiment, IFN-α, IFN-β, IL-1, and IL-1Ra of Table 1 appear to be important in CRS or cytokine storm. In another embodiment, M-CSF has unknown importance. In another embodiment, any cytokine listed in Table 1, or combination thereof, may be used as a marker of CRS or cytokine storm.

TABLE 2
Panel of Cytokines Increased in CRS or Cytokine Storm in Humans and/or Mice

| | Human | Mouse model (pre- | | | |
|---|---|---|---|---|---|
| | model | clinical) | | | |
| Cytokine | (clinical | Mouse | Not | | Notes/ |
| (Analyte) | trials) | origin | specified | Cells secreting this cytokine | other |
| IL-15 | * | | * | Fibroblasts, monocytes (?) | 22 |
| IL-17 | * | | * | T cells | |
| IL-18 | | | | Macrophages | |
| IL-21 | * | | | T helper cells, NK cells | |
| IL-22 | * | | | activated DC and T cells | |
| IL-23 | | | | | |
| IL-25 | | | | | Protective? |
| IL-27 | * | | | APC | |
| IP-10 | * | | | Monocytes (?) | |
| MCP-1 | * | | | Endothel, fibroblast, epithel, | =CXCL10 |
| | | | | monocytes | |
| MCP-3 | * | | | PBMCs, MØ (?) | =CCL2 |
| MIP-1α | * | | * (in vitro) | T cells | =CXCL9 |
| MIP-1β | * | | | T cells | =CCL3 |
| PAF | ? | | | platelets, endothelial cells, | =CCL4 |
| | | | | neutrophils, monocytes, and | |
| | | | | macrophages, mesangial cells | |
| PGE2 | * | | * | Gastrointestinal mucosa and other | |
| RANTES | * | | | Monocytes | |
| TGF-β | * | | * | MØ, lymphocytes, endothel, | =CCL5 |
| | | | | platelets . . . | |
| TNF-α | * | * | * (in vitro) | Macrophages, NK cells, T cells | |
| TNF-αR1 | * | | | | |
| HGF | | | | | |
| MIG | * | | | T cell chemoattractant, induced by | |
| | | | | IFN-γ | |

In one embodiment, IL-15, IL-17, IL-18, IL-21, IL-22, IP-10, MCP-1, MIP-1a, MIP-1(3, and TNF-α of Table 2 are considered to be significant in CRS or cytokine storm. In another embodiment, IL-27, MCP-3, PGE2, RANTES, TGF-β, TNF-αR1, and MIG of Table 2 appear to be important in CRS or cytokine storm. In another embodiment, IL-23 and IL-25 have unknown importance. In another embodiment, any cytokine listed in Table 2, or combination thereof, may be used as a marker of CRS or cytokine storm. In another embodiment, mouse cytokines IL-10, IL-1(3, IL-2, IP-10, IL-4, IL-5, IL-6, IFNα, IL-9, IL-13, IFN-γ, IL-12p70, GM-CSF, TNF-α, MIP-1α, MIP-1β, IL-17A, IL-15/IL-15R and IL-7 appear to be important in CRS or cytokine storm.

A skilled artisan would appreciate that the term “cytokine” may encompass cytokines (e.g., interferon gamma (IFN-γ), granulocyte macrophage colony stimulating factor, tumor necrosis factor alpha), chemokines (e.g., MIP 1 alpha, MIP 1 beta, RANTES), and other soluble mediators of inflammation, such as reactive oxygen species and nitric oxide.

In one embodiment, increased release of a particular cytokine, whether significant, important or having unknown importance, does not a priori mean that the particular cytokine is part of a cytokine storm. In one embodiment, an increase of at least one cytokine is not the result of a cytokine storm or CRS. In some embodiments, an increase of at least one cytokine is as a result of SARS-CoV-2 infection and or symptoms associated with COVID-19

In another embodiment, cytokine release syndrome is characterized by any or all of the following symptoms: Fever with or without rigors, malaise, fatigue, anorexia, myalgias, arthalgias, nausea, vomiting, headache Skin Rash, Nausea, vomiting, diarrhea, Tachypnea, hypoxemia Cardiovascular Tachycardia, widened pulse pressure, hypotension, increased cardiac output (early), potentially diminished cardiac output (late), Elevated D-dimer, hypofibrinogenemia with or without bleeding, Azotemia Hepatic Transaminitis, hyperbilirubinemia, Headache, mental status changes, confusion, delirium, word finding difficulty or frank aphasia, hallucinations, tremor, dymetria, altered gait, seizures. In another embodiment, a cytokine storm is characterized by IL-2 release and lymphoproliferation.

In another embodiment, cytokine storm leads to potentially life-threatening complications including cardiac dysfunction, adult respiratory distress syndrome, neurologic toxicity, renal and/or hepatic failure, and disseminated intravascular coagulation.

A skilled artisan would appreciate that the characteristics of a cytokine release syndrome (CRS) or cytokine storm are estimated to occur a few days to several weeks following the trigger for the CRS or cytokine storm.

In one embodiment, measurement of cytokine levels or concentration, as an indicator of cytokine storm, may be expressed as-fold increase, percent (%) increase, net increase or rate of change in cytokine levels or concentration. In another embodiment, absolute cytokine levels or concentrations above a certain level or concentration may be an indication of a subject undergoing or about to experience a cytokine storm.

A skilled artisan would appreciate that the term “cytokine level” may encompass a measure of concentration, a measure of fold change, a measure of percent (%) change, or a measure of rate change. Further, the methods for measuring cytokines in blood, saliva, serum, urine, and plasma are well known in the art.

In one embodiment, despite the recognition that cytokine storm is associated with elevation of several inflammatory cytokines, IL-6 levels may be used as a common measure of cytokine storm and/or as a common measure of the effectiveness of a treatment for cytokine storms. A skilled artisan would appreciate that other cytokines may be used as markers of a cytokine storm, for example TNF-α, IB-1α, IL-8, IL-13, or INF-γ. Further, that assay methods for measuring cytokines are well known in the art. A skilled artisan would appreciate that methods affecting a cytokine storm may similarly affect cytokine release syndrome.

In one embodiment, disclosed herein is a method of decreasing or inhibiting cytokine production in a subject experiencing cytokine release syndrome or a cytokine storm. In another embodiment, disclosed herein is a method of decreasing or inhibiting cytokine production in a subject vulnerable to experiencing cytokine release syndrome or a cytokine storm. In another embodiment, methods disclosed herein decrease or inhibit cytokine production in a subject experiencing cytokine release syndrome or a cytokine storm, wherein production of any cytokine or group of cytokines listed in Tables 1 and/or 2 is decreased or inhibited. In another embodiment, cytokine IL-6 production is decreased or inhibited. In another embodiment, cytokine IL-beta1 production is decreased or inhibited. In another embodiment, cytokine IL-8 production is decreased or inhibited. In another embodiment, cytokine IL-13 production is decreased or inhibited. In another embodiment, cytokine TNF-alpha production is decreased or inhibited. In another embodiment, cytokines IL-6 production, IL-1beta production, or TNF-alpha production, or any combination thereof is decreased or inhibited.

In one embodiment, cytokine release syndrome is graded. In another embodiment, Grade 1 describes cytokine release syndrome in which symptoms are not life threatening and require symptomatic treatment only, e.g., fever, nausea, fatigue, headache, myalgias, malaise. In another embodiment, Grade 2 symptoms require and respond to moderate intervention, such as oxygen, fluids or vasopressor for hypotension. In another embodiment, Grade 3 symptoms require and respond to aggressive intervention. In another embodiment, Grade 4 symptoms are life-threatening symptoms and require ventilator and patients display organ toxicity.

In another embodiment, a cytokine storm is characterized by IL-6 and interferon gamma release. In another embodiment, a cytokine storm is characterized by release of any cytokine or combination thereof, listed in Tables 1 and 2. In another embodiment, a cytokine storm is characterized by release of any cytokine or combination thereof, known in the art.

In one embodiment, a method of inhibiting or reducing the incidence of a cytokine release syndrome (CRS) or a cytokine storm in a subject comprises administering an early apoptotic cell population or an early apoptotic cell supernatant or compositions thereof. In another embodiment, the early apoptotic cell population or an early apoptotic cell supernatant or compositions thereof may aid in the inhibition or reducing the incidence of the CRS or cytokine storm. In another embodiment, the early apoptotic cell population or an early apoptotic cell supernatant or compositions thereof may aid in treating the CRS or cytokine storm. In another embodiment, the early apoptotic cell population or an early apoptotic cell supernatant or compositions thereof may aid in preventing the CRS or cytokine storm. In another embodiment, the early apoptotic cell population or an early apoptotic cell supernatant or compositions thereof may aid in ameliorating the CRS or cytokine storm. In another embodiment, the apoptotic cell population or an apoptotic cell supernatant or compositions thereof may aid in alleviating the CRS or cytokine storm.

In one embodiment, a method of inhibiting or reducing the incidence of a cytokine release syndrome (CRS) or a cytokine storm in a subject, and being administered an early apoptotic cell population or an early apoptotic cell supernatant or compositions thereof, comprises administering an additional agent. In another embodiment, the additional agent may aid in the inhibition or reducing the incidence of the CRS or cytokine storm. In another embodiment, the additional agent may aid in treating the CRS or cytokine storm. In another embodiment, the additional agent may aid in preventing the CRS or cytokine storm. In another embodiment, the additional agent may aid in ameliorating the CRS or cytokine storm. In another embodiment, the additional agent may aid in alleviating the CRS or cytokine storm.

In one embodiment, a method of inhibiting or reducing the incidence of a cytokine release syndrome (CRS) or a cytokine storm in a subject comprises administering an additional agent. In one embodiment, a method of inhibiting or reducing the incidence of a cytokine release syndrome (CRS) or a cytokine storm in a subject comprises administering an additional agent. In another embodiment, the additional agent may aid the COVID-19 therapy. In one embodiment, a method of inhibiting or reducing the incidence of a cytokine release syndrome (CRS) or a cytokine storm in a subject comprises administering an additional agent.

In another embodiment, the additional agent may aid in the inhibition or reducing the incidence of the CRS or cytokine storm. In another embodiment, the additional agent may aid in treating the CRS or cytokine storm. In another embodiment, the additional agent may aid in preventing the CRS or cytokine storm. In another embodiment, the additional agent may aid in ameliorating the CRS or cytokine storm. In another embodiment, the additional agent may aid in alleviating the CRS or cytokine storm.

In some embodiments, the additional agent for decreasing harmful cytokine release comprises a CTLA-4 blocking agent. In another embodiment, the additional agent for decreasing harmful cytokine release comprises an alpha-1 anti-trypsin or fragment thereof or analogue thereof. In another embodiment, the additional agent for decreasing harmful cytokine release comprises a tellurium-based compound. In another embodiment, the additional agent for decreasing harmful cytokine release comprises an immune modulating agent.

Alpha-1-Antitrypsin (AAT)

Alpha-1-antitrypsin (AAT) is a circulating 52-kDa glycoprotein that is produced mainly by the liver. AAT is primarily known as a serine protease inhibitor and is encoded by the gene SERPINA1. AAT inhibits neutrophil elastase, and inherited deficiency in circulating AAT results in lung-tissue deterioration and liver disease. Serum AAT concentrations in healthy individuals increase twofold during inflammation.

There is a negative association between AAT levels and the severity of several inflammatory diseases. For example, reduced levels or activity of AAT have been described in patients with HIV infection, diabetes mellitus, hepatitis C infection-induced chronic liver disease, and several types of vasculitis.

Increasing evidence demonstrates that human serum derived alpha-1-anti-trypsin (AAT) reduces production of pro-inflammatory cytokines, induces anti-inflammatory cytokines, and interferes with maturation of dendritic cells.

Indeed, the addition of AAT to human peripheral blood mononuclear cells (PBMC) inhibits LPS induced release of TNF-α and IL-10 but increases IL-1 receptor antagonist (IL-1Ra) and IL-10 production.

AAT reduces in vitro IL-1β-mediated pancreatic islet toxicity, and AAT monotherapy prolongs islet allograft survival, promotes antigen-specific immune tolerance in mice, and delays the development of diabetes in non-obese diabetic (NOD) mice. AAT was shown to inhibit LPS-induced acute lung injury in experimental models. Recently, AAT was shown to reduce the size of infarct and the severity of heart failure in a mouse model of acute myocardial ischemia-reperfusion injury.

Monotherapy with clinical-grade human AAT (hAAT) reduced circulating pro-inflammatory cytokines, diminished Graft vs Host Disease (GvHD) severity, and prolonged animal survival after experimental allogeneic bone marrow transfer (Tawara et al., Proc Natl Acad Sci USA. 2012 Jan. 10; 109(2):564-9), incorporated herein by reference. AAT treatment reduced the expansion of alloreactive T effector cells but enhanced the recovery of T regulatory T-cells, (Tregs) thus altering the ratio of donor T effector to T regulatory cells in favor of reducing the pathological process. In vitro, AAT suppressed LPS-induced in vitro secretion of proinflammatory cytokines such as TNF-α and IL-1(3, enhanced the production of the anti-inflammatory cytokine IL-10, and impaired NF-κB translocation in the host dendritic cells. Marcondes, Blood. 2014 (October 30; 124(18):2881-91) incorporated herein by reference show that treatment with AAT not only ameliorated GvHD but also preserved and perhaps even enhanced the graft vs leukemia (GVL) effect.

Tellurium-Based Compounds

Tellurium is a trace element found in the human body. Various tellurium compounds, have immune-modulating properties, and have been shown to have beneficial effects in diverse preclinical and clinical studies. A particularly effective family of tellurium-containing compounds is disclosed for example, in U.S. Pat. Nos. 4,752,614; 4,761,490; 4,764,461 and 4,929,739. The immune-modulating properties of this family of tellurium-containing compounds is described, for example, in U.S. Pat. Nos. 4,962,207, 5,093,135, 5,102,908 and 5,213,899, which are all incorporated by reference as if fully set forth herein.

One promising compound is ammonium trichloro(dioxyethylene-O,O′)tellurate, which is also referred to herein and in the art as AS101. AS101, as a representative example of the family of tellurium-containing compound discussed hereinabove, exhibits antiviral (Nat. Immun. Cell Growth Regul. 7(3):163-8, 1988; AIDS Res Hum Retroviruses. 8(5):613-23, 1992), and tumoricidal activity (Nature 330(6144):173-6, 1987; J. Clin. Oncol. 13(9):2342-53, 1995; J. Immunol. 161(7):3536-42, 1998). Further, AS101 is characterized by low toxicity.

In one embodiment, a composition comprising tellurium-containing immune-modulator compounds may be used in methods disclosed herein, where the tellurium-based compound stimulates the innate and acquired arm of the immune response. For example, it has been shown that AS101 is a potent activator of interferon (IFN) in mice (J. Natl. Cancer Inst. 88(18):1276-84, 1996) and humans (Nat. Immun. Cell Growth Regul. 9(3):182-90, 1990; Immunology 70(4):473-7, 1990; J. Natl. Cancer Inst. 88(18):1276-84, 1996.)

In another embodiment, tellurium-based compounds induce the secretion of a spectrum of cytokines, such as IL-1α, IL-6 and TNF-α.

In another embodiment, a tellurium-based compound comprises a tellurium-based compound known in the art to have immune-modulating properties. In another embodiment, a tellurium-based compound comprises ammonium trichloro(dioxyethylene-O,O′)tellurate. In another embodiment, a tellurium-based compound inhibits or reduces a cytokine release syndrome (CRS) of a cytokine storm.

In one embodiment, a tellurium-based compound inhibits the secretion of at least one cytokine. In another embodiment, a tellurium-based compound reduces the secretion of at least one cytokine.

In another embodiment, disclosed herein is a method of decreasing or inhibiting cytokine production in a subject experiencing cytokine release syndrome or cytokine storm or vulnerable to cytokine release syndrome or cytokine storm, comprising the step of administering a composition comprising a tellurium-based compound to said subject.

In one embodiment, a tellurium-based compound is administered alone to control cytokine release. In another embodiment, both a tellurium-based compound and apoptotic cells or a composition thereof, or apoptotic cell supernatants or a composition thereof, are administered to control cytokine release.

Immuno-Modulatory Agents

A skilled artisan would appreciate that immune-modulating agents may encompass extracellular mediators, receptors, mediators of intracellular signaling pathways, regulators of translation and transcription, as well as immune cells. In one embodiment, an additional agent disclosed herein is an immune-modulatory agent known in the art. In another embodiment, use in the methods disclosed here of an immune-modulatory agent reduces the level of at least one cytokine. In another embodiment, use in the methods disclosed here of an immune-modulatory agent reduces or inhibits CRS or a cytokine storm. In some embodiments, use in the methods disclosed herein of an immune-modulatory agent is for treating, preventing, inhibiting the growth, delaying disease progression, reducing the tumor load, or reducing the incidence of a tumor or a cancer, or any combination thereof.

In one embodiment, an immune-modulatory agent comprises compounds that block, inhibit or reduce the release of cytokines or chemokines. In another embodiment, an immune-modulatory agent comprises compounds that block, inhibit or reduce the release of IL-21 or IL-23, or a combination thereof. In another embodiment, an immune-modulatory agent comprises an antiretroviral drug in the chemokine receptor-5 (CCRS) receptor antagonist class, for example maraviroc. In another embodiment, an immune-modulatory agent comprises an anti-DNAM-1 antibody. In another embodiment, an immune-modulatory agent comprises damage/pathogen-associated molecules (DAMPs/PAMPs) selected from the group comprising heparin sulfate, ATP, and uric acid, or any combination thereof. In another embodiment, an immune-modulatory agent comprises a sialic acid binding Ig-like lectin (Siglecs). In another embodiment, an immune-modulatory agent comprises a cellular mediator of tolerance, for example regulatory CD4⁺ CD25⁺ T cells (Tregs) or invariant natural killer T cells (iNK T-cells). In another embodiment, an immune-modulatory agent comprises dendritic cells. In another embodiment, an immune-modulatory agent comprises monocytes. In another embodiment, an immune-modulatory agent comprises macrophages. In another embodiment, an immune-modulatory agent comprises JAK2 or JAK3 inhibitors selected from the group comprising ruxolitinib and tofacitinib. In another embodiment, an immune-modulatory agent comprises an inhibitor of spleen tyrosine kinase (Syk), for example fostamatinib. In another embodiment, an immune-modulatory agent comprises histone deacetylase inhibitor vorinostat acetylated STAT3. In another embodiment, an immune-modulatory agent comprises neddylation inhibitors, for example MLN4924. In another embodiment, an immune-modulatory agent comprises an miR-142 antagonist. In another embodiment, an immune-modulatory agent comprises a chemical analogue of cytidine, for example Azacitidine. In another embodiment, an immune-modulatory agent comprises an inhibitor of histone deacetylase, for example Vorinostat. In another embodiment, an immune-modulatory agent comprises an inhibitor of histone methylation. In another embodiment, an immune-modulatory agent comprises an antibody. In another embodiment, the antibody is rituximab (RtX)

In another embodiment, compositions and methods as disclosed herein utilize combination therapy of early apoptotic cells with one or more CTLA-4-blocking agents such as Ipilimumab.

In another embodiment, CTLA-4 is a potent inhibitor of T-cell activation that helps to maintain self-tolerance. In another embodiment, administration of an anti-CTLA-4 blocking agent, which in another embodiment, is an antibody, produces a net effect of T-cell activation.

In some embodiment, a viral or bacterial infection causes the cytokine release syndrome or cytokine storm in the subject. In one embodiment, the infection is a SARS-CoV-2 infection. In one embodiment, the infection is an influenza infection. In one embodiment, the influenza infection is H1N1. In another embodiment, the influenza infection is an H5N1 bird flu. In another embodiment, the infection is severe acute respiratory syndrome (SARS). In another embodiment, the subject has Epstein-Barr virus-associated hemophagocytic lymphohistiocytosis (HLH). In another embodiment, the infection is sepsis. In one embodiment, the sepsis is caused by a gram-negative bacterium. In another embodiment, the infection is malaria. In another embodiment, the infection is an Ebola virus infection. In another embodiment, the infection is variola virus. In another embodiment, the infection is a systemic Gram-negative bacterial infection. In another embodiment, the infection is Jarisch-Herxheimer syndrome.

In one embodiment, the cause of the cytokine release syndrome or cytokine storm in a subject is hemophagocytic lymphohistiocytosis (HLH). In another embodiment, HLH is sporadic HLH. In another embodiment, HLH is macrophage activation syndrome (MAS). In another embodiment, the cause of the cytokine release syndrome or cytokine storm in a subject is MAS.

In one embodiment, the cause of the cytokine release syndrome or cytokine storm in a subject is chronic arthritis. In another embodiment, the cause of the cytokine release syndrome or cytokine storm in a subject is systemic Juvenile Idiopathic Arthritis (sJIA), also known as Still's Disease.

In one embodiment, the cause of the cytokine release syndrome or cytokine storm in a subject is Cryopyrin-associated Periodic Syndrome (CAPS). In another embodiment, CAPS comprises Familial Cold Auto-inflammatory Syndrome (FCAS), also known as Familial Cold Urticaria (FCU). In another embodiment, CAPS comprises Muckle-Well Syndrome (MWS). In another embodiment, CAPS comprises Chronic Infantile Neurological Cutaneous and Articular (CINCA) Syndrome. In yet another embodiment, CAPS comprises FCAS, FCU, MWS, or CINCA Syndrome, or any combination thereof. In another embodiment, the cause of the cytokine release syndrome or cytokine storm in a subject is FCAS. In another embodiment, the cause of the cytokine release syndrome or cytokine storm in a subject is FCU. In another embodiment, the cause of the cytokine release syndrome or cytokine storm in a subject is MWS. In another embodiment, the cause of the cytokine release syndrome or cytokine storm in a subject is CINCA Syndrome. In still another embodiment, the cause of the cytokine release syndrome or cytokine storm in a subject is FCAS, FCU, MWS, or CINCA Syndrome, or any combination thereof.

In another embodiment, the cause of the cytokine release syndrome or cytokine storm in a subject is a cryopyrinopathy comprising inherited or de novo gain of function mutations in the NLRP3 gene, also known as the CIASI gene.

In one embodiment, the cause of the cytokine release syndrome or cytokine storm in a subject is a hereditary auto-inflammatory disorder.

In one embodiment, the trigger for the release of inflammatory cytokines is a lipopolysaccharide (LPS), Gram-positive toxins, fungal toxins, glycosylphosphatidylinositol (GPI) or modulation of RIG-1 gene expression.

In another embodiment, the subject has cytokine release syndrome or cytokine storm secondary to receipt of a therapy.

### COVID-19

In some embodiments, disclosed herein is a method of treating COVID-19 in a subject infected by SARS-CoV-2 virus, comprising administering a composition comprising an early apoptotic mononuclear-enriched cell population to said subject, wherein said administering treats COVID-19 in said subject. In some embodiments, a method of treating COVID-19 comprises treating, inhibiting, reducing the incidence of, ameliorating, or alleviating a symptom of COVID-19. In some embodiment, methods of treating comprise treating a COVID-19 subject, wherein said subject additional is of an older age, and or suffering from another disease, for example but not limited to cancer, diabetes, hypertension, cardiovascular disease, chronic respiratory disease, renal disease, and obesity, etc.

Transmission of SARS-CoV-2 virus occurs primarily through respiratory secretions, and, to a lesser extent, contact with contaminated surfaces. Most transmissions are thought to occur through droplets; covering coughs and sneezes.

Following a subject's exposure to the SARS-CoV-2 virus, the estimated incubation period for COVID-19 is up to 14 days from the time of exposure, with a median incubation period of 4 to 5 days. Thus, in some embodiments, treatment of COVID-19 comprising treating a subject prior to the appearance of symptoms. This may be especially important for “at-risk” subject, wherein an at-risk subject comprises someone who may be immune deficient or immune suppressed, or may be suffering from an additional disease. In some embodiments, an at-risk subject is suffering a cancer, diabetes, a lung deficiency, etc.

In some embodiments, COVID-19 comprises an asymptomatic infection, pneumonia, or severe pneumonia with acute respiratory distress syndrome (ARDS). Thus, there is a wide range of COVID-19 symptoms. In some embodiments, COVID-19 is mild comprising no pneumonia or symptoms of mild pneumonia. In some embodiments, COVID-19 is severe comprising symptoms including dyspnea, respiratory frequency ≥30 breaths/min, SpO2≤93%, PaO₂/FiO₂<300 mmHg, and/or lung infiltrates >50% within 24 to 48 hours. In some embodiments, COVID-19 is considered critical comprising symptoms of respiratory failure, septic shock, and/or multiple organ dysfunction or failure.

In some embodiments, organ dysfunction or organ failure comprises lung dysfunction or failure. In some embodiments, organ dysfunction or organ failure comprises lung damage. In some embodiments, lung dysfunction comprises acute respiratory distress syndrome (ARDS). In some embodiments, lung failure comprises respiratory failure. In some embodiments, lung dysfunction comprises pneumonia, which may be mild or severe. In some embodiments, lung dysfunction comprises respiratory complications.

In some embodiments, organ dysfunction or organ failure comprises multiple organ dysfunction or multiple organ failure. In some embodiments, multiple organ dysfunction or failure comprises acute dysfunction or failure. In some embodiments, multiple organ dysfunction or failure comprises chronic dysfunction or failure. In some embodiments, multiple organ dysfunction or failure comprises dysfunction and or failure of at least two organs. In some embodiments, multiple organ dysfunction or failure comprises dysfunction and or failure of at least three organs. In some embodiments, multiple organ dysfunction or failure comprises dysfunction and or failure of at least four organs. In some embodiments, multiple organ dysfunction or failure comprises dysfunction and or failure of a lung, heart, kidney, or liver, or a combination thereof.

In some embodiments, COVID-19 symptoms comprise any of fever, cough, shortness of breath, muscle aches, headaches, diarrhea, dizziness, rhinorrhea, anosmia, dysgeusia, sore throat, abdominal pain, anorexia, vomiting, pneumonia, mild pneumonia, dyspnea, respiratory frequency ≥30 breaths/min, SpO₂≤93%, PaO₂/FiO₂<300 mmHg, lung infiltrates, respiratory distress, ARDS respiratory failure, septic shock, organ dysfunction or failure, or multiple organ dysfunction or failure. In some embodiments, COVID-19 symptoms comprise fever, cough, shortness of breath, muscle aches, headaches, diarrhea, dizziness, rhinorrhea, anosmia, dysgeusia, sore throat, abdominal pain, anorexia, vomiting, pneumonia, mild pneumonia, dyspnea, respiratory frequency ≥30 breaths/min, SpO₂≤93%, PaO₂/FiO₂<300 mmHg, lung infiltrates, respiratory distress, ARDS respiratory failure, septic shock, organ dysfunction or failure, or multiple organ dysfunction or failure, or any combination thereof.

Lung symptoms may be diagnosed using methods well known in the art for example chest X-rays or computed tomography (CT) of the chest.

In some embodiments, a COVID-19 subject comprises a patient experiencing a mild illness defined by a variety of signs and symptoms comprising fever, cough, sore throat, malaise, headache, muscle pain, without shortness of breath, dyspnea on exertion, or abnormal imaging.

In some embodiments, a COVID-19 subject comprises a patient experiencing a moderate COVID-19 illness is defined as evidence of lower respiratory disease by clinical assessment or imaging with SpO₂≥94% on room air at sea level. A skilled clinician would appreciate that pulmonary disease can rapidly progress in patients with moderate COVID-19. If pneumonia or early stages of a cytokine storm are suspected, in some embodiments, methods of use disclosed herein comprising administering an early apoptotic mononuclear-enriched population comprise administration of a composition comprising early apoptotic mononuclear-enriched population as prophylactic measure prior to the appearance of more severe symptoms. Similarly, if pneumonia or early stages of a cytokine storm are suspected, in some embodiments, methods of use disclosed herein comprising administering an early apoptotic supernatant comprise administration of a composition comprising an early apoptotic supernatant as prophylactic measure prior to the appearance of more severe symptoms.

In some embodiments, a COVID-19 subject comprises a patient experiencing a severe illness, wherein said subject has SpO₂<94% on room air at sea level, respiratory rate >30, PaO₂/FiO₂<300 mmHg, or lung infiltrates >50%. A skilled clinician would appreciate that severe COVID-19 can rapidly progress in patients with COVID-19. If pneumonia or early stages of a cytokine storm are suspected or evidenced, in some embodiments, methods of use disclosed herein comprising administering an early apoptotic mononuclear-enriched population comprise administration of a composition comprising early apoptotic mononuclear-enriched population as prophylactic measure prior to the appearance of even more severe symptoms. Similarly, if pneumonia or early stages of a cytokine storm are suspected, in some embodiments, methods of use disclosed herein comprising administering an early apoptotic supernatant comprise administration of a composition comprising an early apoptotic supernatant as prophylactic measure prior to the appearance of even more severe symptoms.

In some embodiments, a COVID-19 subject comprises a patient experiencing a critical illness, wherein said subject is suffering from respiratory failure, septic shock, and/or multiple organ dysfunction or failure. In some embodiments, severe cases of COVID-19 may be associated with acute respiratory distress syndrome, septic shock that may represent virus-induced distributive shock, cardiac dysfunction, elevations in multiple inflammatory cytokines that provoke a cytokine storm, and/or exacerbation of underlying comorbidities. In addition to pulmonary disease, patients with COVID-19 may also experience cardiac, hepatic, renal, and central nervous system disease. A skilled clinician would appreciate that critical COVID-19 can rapidly progress in patients with COVID-19 to death.

The status of COVID-19 may be evaluating using methods well known in the art, for example pulmonary imagining (chest x-ray, ultrasound, or, if indicated, CT) and ECG, if indicated. Laboratory evaluation includes a CBC with differential and a metabolic profile, including liver and renal function tests.

In some embodiments, severe COVID-19 illness comprises acute respiratory distress syndrome, septic shock that may represent virus-induced distributive shock, cardiac dysfunction, elevations in multiple inflammatory cytokines that provoke a cytokine storm, and/or exacerbation of underlying comorbidities. In some embodiments, severe COVID-19 illness comprises pulmonary disease, in combination with cardiac, hepatic, renal, and central nervous system disease.

In some embodiments, critical COVID-19 comprises respiratory failure, septic shock, and/or multiple organ dysfunction or failure. In some embodiments, critical COVID-19 comprises respiratory failure, septic shock, and/or multiple organ dysfunction or failure, in combination with cardiac, hepatic, renal, and central nervous system disease. In some embodiments, critical COVID-19 comprises respiratory failure, septic shock, and/or multiple organ dysfunction or failure, in combination with thromboembolic events.

In some embodiments, a COVID-19 patient may express high levels of an array of inflammatory cytokines, often in the setting of deteriorating hemodynamic or respiratory status. This is often referred to as “cytokine release syndrome” or “cytokine storm,”, as described in detail herein.

In some embodiments, COVID-19 is associated with a potentially severe inflammatory syndrome in children (multisystem inflammatory syndrome in children or MIS-C).

In some embodiments, a COVID-19 patient may experience cardiac dysfunction, including for example but not limited to myocarditis and pericardial dysfunction.

In some embodiments, a severe COVID-19 illness comprises renal and hepatic dysfunction in combination with pulmonary dysfunction and or failure.

In some embodiments, symptoms of COVID-19 disease include sepsis. Thus, methods disclosed herein for treating COVID-19 would further treat, reduce the incidence of, ameliorate, or alleviate sepsis in a subject in need, said methods comprising the step of administering a composition comprising an early apoptotic cell population or supernatant thereof to said subject in combination with an antibiotic, wherein said administering treats, reduces the incidence of, ameliorates, or alleviates sepsis in said subject as well as treating COVID-19.

In some embodiments, sepsis comprises severe sepsis. In some embodiments, sepsis comprises mild sepsis. In some embodiments, sepsis comprises acute sepsis. In some embodiments, sepsis comprises highly aggressive sepsis.

In some embodiments, the source of sepsis comprises pneumonia. In some embodiments, the source of sepsis comprises endovascular Methicillin-resistant Staphylococcus aureus (MRSA). In some embodiments, the source of sepsis comprises a urinary tract infection (UTI). In some embodiments, the source of sepsis comprises a biliary tract infection.

### Apoptotic Cells

In some embodiments, compositions of early apoptotic cells comprise a population of mononuclear apoptotic cell comprising mononuclear cells in an early-apoptotic state, wherein said mononuclear apoptotic cell population comprises: a decreased percent of non-quiescent non-apoptotic viable cells; a suppressed cellular activation of any living non-apoptotic cells; or a reduced proliferation of any living non-apoptotic cells; or any combination thereof.

This disclosure provides in some embodiments, a pooled mononuclear apoptotic cell preparation comprising mononuclear cells in an early apoptotic state, wherein said pooled mononuclear apoptotic cells preparation comprises pooled individual mononuclear cell populations, and wherein said pooled mononuclear apoptotic cell preparation comprises a decreased percent of living non-apoptotic cells, a suppressed cellular activation of any living non-apoptotic cells, or a reduced proliferation of any living non-apoptotic cells, or any combination thereof. In another embodiment, the pooled mononuclear apoptotic cells have been irradiated. In another embodiment, this disclosure provides a pooled mononuclear apoptotic cell preparation that in some embodiments, uses the white blood cell fraction (WBC) obtained from donated blood. Often this WBC fraction is discarded at blood banks or is targeted for use in research.

In some embodiments, a cell population disclosed herein is inactivated. In another embodiment, inactivation comprises irradiation. In another embodiment, inactivation comprises T-cell receptor inactivation. In another embodiment, inactivation comprises T-cell receptor editing. In another embodiment, inactivation comprises suppressing or eliminating an immune response in said preparation. In another embodiment, inactivation comprises suppressing or eliminating cross-reactivity between multiple individual populations comprised in the preparation. In other embodiment, inactivation comprises reducing or eliminating T-cell receptor activity between multiple individual populations comprised in the preparation. In another embodiment, an inactivated cell preparation comprises a decreased percent of living non-apoptotic cells, suppressed cellular activation of any living non-apoptotic cells, or a reduce proliferation of any living non-apoptotic cells, or any combination thereof.

In another embodiment, an inactivated cell population comprises a reduced number of non-quiescent non-apoptotic cells compared with a non-radiated cell preparation. In some embodiments, an inactivated cell population comprises 50 percent (%) of living non-apoptotic cells. In some embodiments, an inactivated cell population comprises 40% of living non-apoptotic cells. In some embodiments, an inactivated cell population comprises 30% of living non-apoptotic cells. In some embodiments, an inactivated cell population comprises 20% of living non-apoptotic cells. In some embodiments, an inactivated cell population comprises 100% of living non-apoptotic cells. In some embodiments, an inactivated cell population comprises 0% of living non-apoptotic cells.

In some embodiments, disclosed herein is a method of preparing an inactivated early apoptotic cell population. In some embodiments, disclosed herein is a method for producing a population of mononuclear apoptotic cell comprising a decreased percent of non-quiescent non-apoptotic viable cells; a suppressed cellular activation of any living non-apoptotic cells; or a reduced proliferation of any living non-apoptotic cells; or any combination thereof, said method comprising the following steps: obtaining a mononuclear-enriched cell population of peripheral blood; freezing said mononuclear-enriched cell population in a freezing medium comprising an anticoagulant; thawing said mononuclear-enriched cell population; incubating said mononuclear-enriched cell population in an apoptosis inducing incubation medium comprising methylprednisolone at a final concentration of about 10-100 μg/mL and an anticoagulant; resuspending said apoptotic cell population in an administration medium; and inactivating said mononuclear-enriched population, wherein said inactivation occurs following induction, wherein said method produces a population of mononuclear apoptotic cell comprising a decreased percent of non-quiescent non-apoptotic cells; a suppressed cellular activation of any living non-apoptotic cells; or a reduced proliferation of any living non-apoptotic cells; or any combination thereof. In some embodiments, early apoptotic mononuclear-cell-enriched population comprises early apoptotic cells irradiated after induction of apoptosis.

In another embodiment, the irradiation comprises gamma irradiation or UV irradiation. In yet another embodiment, the irradiated preparation has a reduced number of non-quiescent non-apoptotic cells compared with a non-irradiated cell preparation.

In another embodiment, the pooled mononuclear apoptotic cells have undergone T-cell receptor inactivation. In another embodiment, the pooled mononuclear apoptotic cells have undergone T-cell receptor editing.

In some embodiments, pooled blood comprises 3^{rd} party blood from HLA matched or HLA unmatched sources, with respect to a recipient.

In certain embodiments, early apoptotic mononuclear-cell-enriched population used in the methods described herein, comprises (a) an apoptotic population stable for greater than 24 hours; (b) an apoptotic population comprising a decreased number of non-quiescent non-apoptotic cells, a suppressed cellular activation of any living non-apoptotic cells, or a reduced proliferation of any living non-apoptotic cells, or (c) a pooled population of early apoptotic mononuclear-enriched cells, or (d) any combination thereof.

Production of apoptotic cells (“ApoCells”) for use in compositions and methods as disclosed herein, has been described in WO 2014/087408, which is incorporated by reference herein in its entirety, and is described in brief in Example 1 below. In another embodiment, early apoptotic cells for use in compositions and methods as disclosed herein are produced in any way that is known in the art. In another embodiment, early apoptotic cells for use in compositions and methods disclosed herein are autologous with a subject undergoing therapy. In another embodiment, early apoptotic cells for use in compositions and methods disclosed herein are allogeneic with a subject undergoing therapy. In another embodiment, a composition comprising early cells comprises apoptotic cells as disclosed herein or as is known in art.

A skilled artisan would appreciate that the term “autologous” may encompass a tissue, cell, nucleic acid molecule or polypeptide in which the donor and recipient is the same person.

A skilled artisan would appreciate that the term “allogeneic” may encompass a tissue, cell, nucleic acid molecule or polypeptide that is derived from separate individuals of the same species. In some embodiments, allogeneic donor cells are genetically distinct from the recipient.

In some embodiments, obtaining a mononuclear-enriched cell composition according to the production method disclosed herein is effected by leukapheresis. A skilled artisan would appreciate that the term “leukapheresis” may encompass an apheresis procedure in which leukocytes are separated from the blood of a donor. In some embodiments, the blood of a donor undergoes leukapheresis and thus a mononuclear-enriched cell composition is obtained according to the production method disclosed herein. It is to be noted, that the use of at least one anticoagulant during leukapheresis is required, as is known in the art, in order to prevent clotting of the collected cells.

In some embodiments, the leukapheresis procedure is configured to allow collection of mononuclear-enriched cell composition according to the production method disclosed herein. In some embodiments, cell collections obtained by leukapheresis comprise at least 65% mononuclear cells. In other embodiments, cell collections obtained by leukapheresis comprise at least at least 70%, or at least 80% mononuclear cells. In some embodiments, blood plasma from the cell-donor is collected in parallel to obtaining of the mononuclear-enriched cell composition. In some embodiments, about 300-600 ml of blood plasma from the cell-donor are collected in parallel to obtaining the mononuclear-enriched cell composition according to the production method disclosed herein. In some embodiments, blood plasma collected in parallel to obtaining the mononuclear-enriched cell composition according to the production method disclosed herein is used as part of the freezing and/or incubation medium. Additional detailed methods of obtaining an enriched population of apoptotic cells for use in the compositions and methods as disclosed herein may be found in WO 2014/087408, which is incorporated herein by reference in its entirety.

In some embodiments, the early apoptotic cells for use in the methods disclosed herein comprise at least 85% mononuclear cells. In further embodiments, the early apoptotic cells for use in the methods disclosed herein contains at least 85% mononuclear cells, 90% mononuclear cells or alternatively over 90% mononuclear cells. In some embodiments, the early apoptotic cells for use in the methods disclosed herein comprise at least 90% mononuclear cells. In some embodiments, the early apoptotic cells for use in the methods disclosed herein comprise at least 95% mononuclear cells.

It is to be noted that, in some embodiments, while the mononuclear-enriched cell preparation at cell collection comprises at least 65%, preferably at least 70%, most preferably at least 80% mononuclear cells, the final pharmaceutical population, following the production method of the early apoptotic cells for use in the methods disclosed herein, comprises at least 85%, preferably at least 90%, most preferably at least 95% mononuclear cells.

In certain embodiments, the mononuclear-enriched cell preparation used for production of the composition of the early apoptotic cells for use in the methods disclosed herein comprises at least 50% mononuclear cells at cell collection. In certain embodiments, disclosed herein is a method for producing the pharmaceutical population wherein the method comprises obtaining a mononuclear-enriched cell preparation from the peripheral blood of a donor, the mononuclear-enriched cell preparation comprising at least 50% mononuclear cells. In certain embodiments, disclosed herein is a method for producing the pharmaceutical population wherein the method comprises freezing a mononuclear-enriched cell preparation comprising at least 50% mononuclear cells.

In some embodiments, the cell preparation comprises at least 85% mononuclear cells, wherein at least 40% of the cells in the preparation are in an early-apoptotic state, wherein at least 85% of the cells in the preparation are viable cells. In some embodiments, the apoptotic cell preparation comprises no more than 15% CD15^{high} expressing cells.

A skilled artisan would appreciate that the term “early-apoptotic state” may encompass cells that show early signs of apoptosis without late signs of apoptosis. Examples of early signs of apoptosis in cells include exposure of phosphatidylserine (PS) and the loss of mitochondrial membrane potential. Examples of late events include propidium iodide (PI) admission into the cell and the final DNA cutting. In order to document that cells are in an “early apoptotic” state, in some embodiments, PS exposure detection by Annexin-V and PI staining are used, and cells that are stained with Annexin V but not with PI or with low PI staining are considered to be “early apoptotic cells” (An⁺PI⁻). In some embodiments, minimal PI staining comprising less than or equal to (≤)15% PI+ cells within the population of cells. In some embodiments, minimal PI staining comprising ≤10% PI+ cells within the population of cells. In some embodiments, minimal PI staining comprising ≤5% PI+ cells within the population of cells.

In another embodiment, cells that are stained by both Annexin-V FITC and high PI are considered to be “late apoptotic cells”. In some embodiments, high PI staining comprises greater than (>) 15% PI+ cells within the population of cells. In some embodiments, high PI staining comprises greater than or equal to (≥) 16% PI+ cells within the population of cells. In another embodiment, cells that do not stain for either Annexin-V or PI are considered non-apoptotic viable cells.

In some embodiments, at least 40% of the cells in a preparation are in an early apoptotic state. In some embodiments, at least 45% of the cells in a preparation are in an early apoptotic state. In some embodiments, at least 50% of the cells in a preparation are in an early apoptotic state. In some embodiments, at least 55% of the cells in a preparation are in an early apoptotic state. In some embodiments, at least 60% of the cells in a preparation are in an early apoptotic state. In some embodiments, at least 65% of the cells in a preparation are in an early apoptotic state. In some embodiments, at least 70% of the cells in a preparation are in an early apoptotic state. In some embodiments, at least 75% of the cells in a preparation are in an early apoptotic state. In some embodiments, at least 80% of the cells in a preparation are in an early apoptotic state. In some embodiments, at least 85% of the cells in a preparation are in an early apoptotic state. In some embodiments, at least 90% of the cells in a preparation are in an early apoptotic state. In some embodiments, at least 95% of the cells in a preparation are in an early apoptotic state.

In some embodiments, an early apoptotic cell preparation comprises less than or equal to (≤) 15% PI⁺ cells. In some embodiments, an early apoptotic cell preparation comprises ≤10% PI⁺ cells. In some embodiments, an early apoptotic cell preparation comprises ≤9% PI⁺ cells. In some embodiments, an early apoptotic cell preparation comprises ≤8% PI⁺ cells. In some embodiments, an early apoptotic cell preparation comprises ≤7% PI⁺ cells. In some embodiments, an early apoptotic cell preparation comprises ≤6% PI⁺ cells. In some embodiments, an early apoptotic cell preparation comprises ≤5% PI⁺ cells. In some embodiments, an early apoptotic cell preparation comprises ≤4% PI⁺ cells. In some embodiments, an early apoptotic cell preparation comprises ≤3% PI⁺ cells. In some embodiments, an early apoptotic cell preparation comprises ≤2% PI⁺ cells. In some embodiments, an early apoptotic cell preparation comprises ≤1% PI⁺ cells.

In some embodiments, at least 40% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 45% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 50% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 55% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 60% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 65% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 70% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 75% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 80% of the cells in a preparation are in an early apoptotic state (An⁺), wherein ≤15%, 10%, 9%, 8%, 7%, 6%, 5%, 4%, 3%, 2%, 1%, or 0% of the cells are PI⁺. In some embodiments, at least 85% of the cells in a preparation are in an early apoptotic state (An⁺), wherein <15% or ≤14%, 10%, 9%, 8%, 7%, 6%, 5%, 4%, 3%, 2%, 1%, or 0% of the cells are PI⁺. In some embodiments, at least 90% of the cells in a preparation are in an early apoptotic state (An⁺), wherein <10%, or ≤9%, 8%, 7%, 6%, 5%, 4%, 3%, 2%, 1%, or 0% of the cells are PI⁺. In some embodiments, at least 95% of the cells in a preparation are in an early apoptotic state (An⁺), wherein <5%, ≤4%, 3%, 2%, 1%, or 0% of the cells are PI⁺.

A skilled artisan would appreciate that in some embodiments the terms “early apoptotic cells”, “early apoptotic mononuclear-enriched cell”, “apoptotic cell”, “Allocetra”, “ALC”, and “ApoCell”, and grammatical variants thereof, may be used interchangeably having all the same qualities and meanings. The skilled artisan would appreciate that the compositions and methods described herein, in some embodiments comprise early apoptotic cells. In some embodiments, as described herein, early apoptotic cells are HLA matched to a recipient (a subject in need of a composition comprising the early apoptotic cells). In some embodiments, as described herein, early apoptotic cells are not matched to a recipient (a subject in need of a composition comprising the early apoptotic cells. In some embodiments, early apoptotic cells are unmatched from a foreign donor. In some embodiments, the early apoptotic cells not matched to a recipient of a composition comprising the early apoptotic cells (a subject in need) are irradiated as described herein in detail. In some embodiments, irradiated not matched cells are termed “Allocetra-OTS” or “ALC-OTS”.

In some embodiments, early apoptotic cells comprise cells in an early apoptotic state. In another embodiment, early apoptotic cells comprise cells wherein at least 90% of said cells are in an early apoptotic state. In another embodiment, early apoptotic cells comprise cells wherein at least 80% of said cells are in an early apoptotic state. In another embodiment, early apoptotic cells comprise cells wherein at least 70% of said cells are in an early apoptotic state. In another embodiment, early apoptotic cells comprise cells wherein at least 60% of said cells are in an early apoptotic state. In another embodiment, early apoptotic cells comprise cells wherein at least 50% of said cells are in an early apoptotic state.

In some embodiments, the composition comprising early cells further comprises an anti-coagulant.

In some embodiments, early apoptotic cells are stable. A skilled artisan would appreciate that in some embodiments, stability encompasses maintaining early apoptotic cell characteristics over time, for example, maintaining early apoptotic cell characteristics upon storage at about 2-8° C. In some embodiments, stability comprises maintaining early apoptotic cell characteristic upon storage at freezing temperatures, for example temperatures at or below 0° C.

In some embodiments, the mononuclear-enriched cell population obtained according to the production method of the early apoptotic cells for use in the methods disclosed herein undergoes freezing in a freezing medium. In some embodiments, the freezing is gradual. In some embodiments, following collection the cells are maintained at room temperature until frozen. In some embodiments, the cell-preparation undergoes at least one washing step in washing medium following cell-collection and prior to freezing.

As used herein, the terms “obtaining cells” and “cell collection” may be used interchangeably. In some embodiments, the cells of the cell preparation are frozen within 3-6 hours of collection. In some embodiments, the cell preparation is frozen within up to 6 hours of cell collection. In some embodiments, the cells of the cell preparations are frozen within 1, 2, 3, 4, 5, 6, 7, 8 hours of collection. In other embodiments, the cells of the cell preparations are frozen up to 8, 12, 24, 48, 72 hours of collection. In other embodiments, following collection the cells are maintained at 2-8° C. until frozen.

In some embodiments, freezing according to the production of an early apoptotic cell population comprises: freezing the cell preparation at about −18° C. to −25° C. followed by freezing the cell preparation at about −80° C. and finally freezing the cell preparation in liquid nitrogen until thawing. In some embodiments, the freezing according to the production of an early apoptotic cell population comprises: freezing the cell preparation at about −18° C. to −25° C. for at least 2 hours, freezing the cell preparation at about −80° C. for at least 2 hours and finally freezing the cell preparation in liquid nitrogen until thawing. In some embodiments, the cells are kept in liquid nitrogen for at least 8, 10 or 12 hours prior to thawing. In some embodiments, the cells of the cell preparation are kept in liquid nitrogen until thawing and incubation with apoptosis-inducing incubation medium. In some embodiments, the cells of the cell preparation are kept in liquid nitrogen until the day of hematopoietic stem cell transplantation. In non-limiting examples, the time from cell collection and freezing to preparation of the final population may be between 1-50 days, alternatively between 6-30 days. In alternative embodiments, the cell preparation may be kept in liquid nitrogen for longer time periods, such as at least several months.

In some embodiments, the freezing according to the production of an early apoptotic cell population comprises freezing the cell preparation at about −18° C. to −25° C. for at least 0.5, 1, 2, 4 hours. In some embodiments, the freezing according to the production of an early apoptotic cell population comprises freezing the cell preparation at about −18° C. to −25° C. for about 2 hours. In some embodiments, the production of an early apoptotic cell population comprises freezing the cell preparation at about −80° C. for at least 0.5, 1, 2, 4, 12 hours.

In some embodiments, the mononuclear-enriched cell composition may remain frozen at least 1, 2, 3, 4, 5, 6, 7, 8, 9, 10, 11, 12, 20 months. In some embodiments, the mononuclear-enriched cell composition may remain frozen at least 0.5, 1, 2, 3, 4, 5 years. In certain embodiments, the mononuclear-enriched cell composition may remain frozen for at least 20 months.

In some embodiments, the mononuclear-enriched cell composition is frozen for at least 8, 10, 12, 18, 24 hours. In certain embodiments, freezing the mononuclear-enriched cell composition is for a period of at least 8 hours. In some embodiments, the mononuclear-enriched cell composition is frozen for at least about 10 hours. In some embodiments, the mononuclear-enriched cell composition is frozen for at least about 12 hours. In some embodiments, the mononuclear-enriched cell composition is frozen for about 12 hours. In some embodiments, the total freezing time of the mononuclear-enriched cell composition (at about −18° C. to −25° C., at about −80° C. and in liquid nitrogen) is at least 8, 10, 12, 18, 24 hours.

In some embodiments, the freezing at least partly induces the early-apoptotic state in the cells of the mononuclear-enriched cell composition. In some embodiments, the freezing medium comprises RPMI 1640 medium comprising L-glutamine, Hepes, Hes, dimethyl sulfoxide (DMSO) and plasma. In some embodiments, the plasma in the freezing medium is an autologous plasma of the donor which donated the mononuclear-enriched cells of the population. In some embodiments, the freezing medium comprises RPMI 1640 medium comprising 2 mM L-glutamine, 10 mM Hepes, 5% Hes, 10% dimethyl sulfoxide and 20% v/v plasma.

In some embodiments, the freezing medium comprises an anti-coagulant. In certain embodiments, at least some of the media used during the production of an early apoptotic cell population, including the freezing medium, the incubation medium and the washing media comprise an anti-coagulant. In certain embodiments, all media used during the production of an early apoptotic cell population which comprise an anti-coagulant comprise the same concentration of anti-coagulant. In some embodiments, anti-coagulant is not added to the final suspension medium of the cell population.

In some embodiments, addition of an anti-coagulant at least to the freezing medium improves the yield of the cell-preparation. In other embodiments, addition of an anti-coagulant to the freezing medium improves the yield of the cell-preparation in the presence of a high triglyceride level. As used herein, improvement in the yield of the cell-preparation relates to improvement in at least one of: the percentage of viable cells out of cells frozen, the percentage of early-state apoptotic cells out of viable cells and a combination thereof.

In some embodiments, early apoptotic cells are stable for at least 24 hours. In another embodiment, early apoptotic cells are stable for 24 hours. In another embodiment, early apoptotic cells are stable for more than 24 hours. In another embodiment, early apoptotic cells are stable for at least 36 hours. In another embodiment, early apoptotic cells are stable for 48 hours. In another embodiment, early apoptotic cells are stable for at least 36 hours. In another embodiment, early apoptotic cells are stable for more than 36 hours. In another embodiment, early apoptotic cells are stable for at least 48 hours. In another embodiment, early apoptotic cells are stable for 48 hours. In another embodiment, early apoptotic cells are stable for at least 48 hours. In another embodiment, early apoptotic cells are stable for more than 48 hours. In another embodiment, early apoptotic cells are stable for at least 72 hours. In another embodiment, early apoptotic cells are stable for 72 hours. In another embodiment, early apoptotic cells are stable for more than 72 hours.

A skilled artisan would appreciate that the term “stable” encompasses apoptotic cells that remain PS-positive (Phosphatidylserine-positive) with only a very small percent of PI-positive (Propidium iodide-positive). PI-positive cells provide an indication of membrane stability wherein a PI-positive cells permits admission into the cells, showing that the membrane is less stable. In some embodiments, stable early apoptotic cells remain in early apoptosis for at least 24 hours, for at least 36 hours, for at least 48 hours, or for at least 72 hours. In another embodiment, stable early apoptotic cells remain in early apoptosis for 24 hours, for 36 hours, for 48 hours, or for 72 hours. In another embodiment, stable early apoptotic cells remain in early apoptosis for more than 24 hours, for more than 36 hours, for more than 48 hours, or for more than 72 hours. In another embodiment, stable early apoptotic cells maintain their state for an extended time period.

In some embodiments, an apoptotic cell population is devoid of cell aggregates. In some embodiments, an apoptotic cell population is devoid of large cell aggregates. In some embodiments, an apoptotic cell population has a reduced number of cell aggregates compared to an apoptotic cell population prepared without adding an anticoagulant in a step other than cell collection (leukapheresis) from the donor. In some embodiments, an apoptotic cell population or a composition thereof, comprises an anticoagulant.

In some embodiments, early apoptotic cells are devoid of cell aggregates, wherein said apoptotic cells were obtained from a subject with high blood triglycerides. In some embodiments, blood triglycerides levels of the subject are above 150 mg/dL. In some embodiments, an apoptotic cell population is devoid of cell aggregates, wherein said apoptotic cell population is prepared from cells obtained from a subject with normal blood triglycerides. In some embodiments, blood triglycerides levels of the subject are equal to or below 150 mg/dL. In some embodiments, cell aggregates produce cell loss during apoptotic cell production methods.

A skilled artisan would appreciate that the terms “aggregates” or “cell aggregates” may encompass the reversible clumping of blood cells under low shear forces or at stasis. Cell aggregates can be visually observed during the incubation steps of the production of the apoptotic cells. Cell aggregation can be measured by any method known in the art, for example by visually imaging samples under a light microscope or using flow cytometry.

In some embodiments, the anti-coagulant is selected from the group comprising: heparin, acid citrate dextrose (ACD) Formula A and a combination thereof. In some embodiments, the anti-coagulant is selected from the group consisting of: heparin, acid citrate dextrose (ACD) Formula A and a combination thereof.

In some embodiments of methods of preparing an early apoptotic cell population and compositions thereof, an anticoagulant is added to at least one medium used during preparation of the population. In some embodiments, the at least one medium used during preparation of the population is selected from the group consisting of: the freezing medium, the washing medium, the apoptosis inducing incubation medium, and any combinations thereof.

In some embodiments, the anti-coagulant is selected from the group consisting of: Heparin, ACD Formula A and a combination thereof. It is to be noted that other anti-coagulants known in the art may be used, such as, but not limited to Fondaparinaux, Bivalirudin and Argatroban.

In some embodiments, at least one medium used during preparation of the population contains 5% of ACD formula A solution comprising 10 U/ml heparin. In some embodiments, anti-coagulant is not added to the final suspension medium of the cell population. As used herein, the terms “final suspension medium” and “administration medium” are used interchangeably having all the same qualities and meanings.

In some embodiments, at least one medium used during preparation of the population comprises heparin at a concentration of between 0.1-2.5 U/ml. In some embodiments, at least one medium used during preparation of the population comprises ACD Formula A at a concentration of between 1%-15% v/v. In some embodiments, the freezing medium comprises an anti-coagulant. In some embodiments, the incubation medium comprises an anti-coagulant. In some embodiments, both the freezing medium and incubation medium comprise an anti-coagulant. In some embodiments the anti-coagulant is selected from the group consisting of: heparin, ACD Formula A and a combination thereof.

In some embodiments, the heparin in the freezing medium is at a concentration of between 0.1-2.5 U/ml. In some embodiments, the ACD Formula A in the freezing medium is at a concentration of between 1%-15% v/v. In some embodiments, the heparin in the incubation medium is at a concentration of between 0.1-2.5 U/ml. In some embodiments, the ACD Formula A in the incubation medium is at a concentration of between 1%-15% v/v. In some embodiments, the anticoagulant is a solution of acid-citrate-dextrose (ACD) formula A. In some embodiments, the anticoagulant added to at least one medium used during preparation of the population is ACD Formula A containing heparin at a concentration of 10 U/ml.

In some embodiments, the apoptosis inducing incubation medium used in the production of an early apoptotic cell population comprises an anti-coagulant. In some embodiments, both the freezing medium and apoptosis inducing incubation medium used in the production of an early apoptotic cell population comprise an anti-coagulant. Without wishing to be bound by any theory or mechanism, in order to maintain a high and stable cell yield in different cell compositions, regardless of the cell collection protocol, in some embodiments addition of anti-coagulants comprising adding the anticoagulant to both the freezing medium and the apoptosis inducing incubation medium during production of the apoptotic cell population. In some embodiments, a high and stable cell yield within the composition comprises a cell yield of at least 30%, preferably at least 40%, typically at least 50% cells of the initial population of cells used for induction of apoptosis.

In some embodiments, both the freezing medium and the incubation medium comprise an anti-coagulant. In some embodiments, addition of an anti-coagulant both to the incubation medium and freezing medium results in a high and stable cell-yield between different preparations of the population regardless of cell-collection conditions, such as, but not limited to, the timing and/or type of anti-coagulant added during cell collection. In some embodiments, addition of an anti-coagulant both to the incubation medium and freezing medium results in a high and stable yield of the cell-preparation regardless of the timing and/or type of anti-coagulant added during leukapheresis. In some embodiments, production of the cell-preparation in the presence of a high triglyceride level results in a low and/or unstable cell-yield between different preparations. In some embodiments, producing the cell-preparation from the blood of a donor having high triglyceride level results in a low and/or unstable cell-yield of the cell preparation. In some embodiments, the term “high triglyceride level” refers to a triglyceride level which is above the normal level of a healthy subject of the same sex and age. In some embodiments, the term “high triglyceride level” refers to a triglyceride level above about 1.7 milimole/liter. As used herein, a high and stable yield refers to a cell yield in the population which is high enough to enable preparation of a dose which will demonstrate therapeutic efficiency when administered to a subject. In some embodiments, therapeutic efficiency refers to the ability to treat, prevent or ameliorate an immune disease, an autoimmune disease or an inflammatory disease in a subject. In some embodiments, a high and stable cell yield is a cell yield of at least 30%, possibly at least 40%, typically at least 50% of cells in the population out of cells initially frozen.

In some embodiments, in case the cell-preparation is obtained from a donor having a high triglyceride level, the donor will take at least one measure selected from the group consisting of: taking triglyceride-lowering medication prior to donation, such as, but not limited to: statins and/or bezafibrate, fasting for a period of at least 8, 10, 12 hours prior to donation, eating an appropriate diet to reduce blood triglyceride level at least 24, 48, 72 hours prior to donating and any combination thereof.

In some embodiments, cell yield in the population relates to cell number in the composition out of the initial number of cells subjected to apoptosis induction. As used herein, the terms “induction of early apoptotic state” and “induction of apoptosis” may be used interchangeably.

In some embodiments, the mononuclear-enriched cell composition is incubated in incubation medium following freezing and thawing. In some embodiments, there is at least one washing step between thawing and incubation. As used herein, the terms “incubation medium” and “apoptosis inducing incubation medium” are used interchangeably. In some embodiments, the incubation medium comprises RPMI 1640 medium supplemented with L-glutamine, Hepes methylprednisolone and plasma. In some embodiments, the washing medium comprises 2 mM L-glutamine, 10 mM Hepes and 10% v/v blood plasma. In some embodiments, the blood plasma in in the incubation medium is derived from the same donor from whom the cells of the cell preparations are derived. In some embodiments, the blood plasma is added to the incubation medium on the day of incubation. In some embodiments, incubation is performed at 37° C. and 5% CO2.

In some embodiments, the incubation medium comprises methylprednisolone. In some embodiments, the methylprednisolone within the incubation medium further induces the cells in the mononuclear-enriched cell composition to enter an early-apoptotic state. In some embodiments, the cells in the mononuclear-enriched cell composition are induced to enter an early-apoptotic state both by freezing and incubating in the presence of methylprednisolone. In some embodiments, the production of an early apoptotic cell population advantageously allows induction of an early-apoptosis state substantially without induction of necrosis, wherein the cells remain stable at said early-apoptotic state for about 24 hours following preparation.

In some embodiments, the incubation medium comprises methylprednisolone at a concentration of about 10-100 μg/ml. In some embodiments, the incubation medium comprises methylprednisolone at a concentration of about 40-60 μg/ml, alternatively about 45-55 μg/ml. In some embodiments, the incubation medium comprises methylprednisolone at a concentration of 50 μg/ml.

In some embodiments, the incubation is for about 2-12 hours, possibly 4-8 hours, typically for about 5-7 hours. In some embodiments, the incubation is for about 6 hours. In some embodiments, the incubation is for at least 6 hours. In a preferred embodiment, the incubation is for 6 hours.

In some embodiments, the incubation medium comprises an anti-coagulant. In some embodiments, addition of an anti-coagulant to the incubation medium improves the yield of the cell-preparation. In some embodiments, the anti-coagulant in the incubation medium is of the same concentration as within the freezing medium. In some embodiments, the incubation medium comprises an anti-coagulant selected from the group consisting of: heparin, ACD Formula A and a combination thereof. In some embodiments, the anti-coagulant used in the incubation medium is ACD Formula A containing heparin at a concentration of 10 U/ml.

In some embodiments, the incubation medium comprises heparin. In some embodiments, the heparin in the incubation medium is at a concentration of between 0.1-2.5 U/ml. In some embodiments, the heparin in the incubation medium is at a concentration of between 0.1-2.5 U/ml, possibly between 0.3-0.7 U/ml, typically about 0.5 U/ml. In certain embodiments, the heparin in the incubation medium is at a concentration of about 0.5 U/ml.

In some embodiments, the incubation medium comprises ACD Formula A. In some embodiments, the ACD Formula A in the incubation medium is at a concentration of between 1%-15% v/v. In some embodiments, the ACD Formula A in the incubation medium is at a concentration of between 1%-15% v/v, possibly between 4%-7% v/v, typically about 5% v/v. In some embodiments, the ACD Formula A in the incubation medium is at a concentration of about 5% v/v.

In some embodiments, improvement in the yield of the cell-preparation comprises improvement in the number of the early-apoptotic viable cells of the preparation out of the number of frozen cells from which the preparation was produced.

In some embodiments, addition of an anti-coagulant to the freezing medium contributes to a high and stable yield between different preparations of the pharmaceutical population. In preferable embodiments, addition of an anti-coagulant at least to the freezing medium and incubation medium results in a high and stable yield between different preparations of the pharmaceutical composition, regardless to the cell collection protocol used.

In some embodiments, the freezing medium comprises an anti-coagulant selected from the group consisting of: heparin, ACD Formula A and a combination thereof. In some embodiments, the anti-coagulant used in the freezing medium is ACD Formula A containing heparin at a concentration of 10 U/ml. In some embodiments, the freezing medium comprises 5% v/v of ACD Formula A solution comprising heparin at a concentration of 10 U/ml.

In some embodiments, the freezing medium comprises heparin. In some embodiments, the heparin in the freezing medium is at a concentration of between 0.1-2.5 U/ml. In some embodiments, the heparin in the freezing medium is at a concentration of between 0.1-2.5 U/ml, possibly between 0.3-0.7 U/ml, typically about 0.5 U/ml. In certain embodiments, the heparin in the freezing medium is at a concentration of about 0.5 U/ml.

In some embodiments, the freezing medium comprises ACD Formula A. In some embodiments, the ACD Formula A in the freezing medium is at a concentration of between 1%-15% v/v. In some embodiments, the ACD Formula A in the freezing medium is at a concentration of between 1%-15% v/v, possibly between 4%-7% v/v, typically about 5% v/v. In some embodiments, the ACD Formula A in the freezing medium is at a concentration of about 5% v/v.

In some embodiments, addition of an anti-coagulant to the incubation medium and/or freezing medium results in a high and stable cell yield within the population regardless of the triglyceride level in the blood of the donor. In some embodiments, addition of an anti-coagulant to the incubation medium and/or freezing medium results in a high and stable cell yield within the composition disclosed herein when obtained from the blood of a donor having normal or high triglyceride level. In some embodiments, addition of an anti-coagulant at least to the incubation medium, results in a high and stable cell yield within the composition regardless of the triglyceride level in the blood of the donor. In some embodiments, addition of an anti-coagulant to the freezing medium and incubation medium results in a high and stable cell yield within the composition regardless of the triglyceride level in the blood of the donor.

In some embodiments, the freezing medium and/or incubation medium and/or washing medium comprise heparin at a concentration of at least 0.1 U/ml, possibly at least 0.3 U/ml, typically at least 0.5 U/ml. In some embodiments, the freezing medium and/or incubation medium and/or washing medium comprise ACD Formula A at a concentration of at least 1% v/v, possibly at least 3% v/v, typically at least 5% v/v.

In some embodiments, the mononuclear-enriched cell composition undergoes at least one washing step following cell collection and prior to being re-suspended in the freezing medium and frozen. In some embodiments, the mononuclear-enriched cell composition undergoes at least one washing step following freezing and thawing. In some embodiments, washing steps comprise centrifugation of the mononuclear-enriched cell composition followed by supernatant extraction and re-suspension in washing medium.

In some embodiments, the mononuclear-enriched cell composition undergoes at least one washing step between each stage of the production of an early apoptotic cell population. In some embodiments, anti-coagulant is added to washing media during washing steps throughout the production of an early apoptotic cell population. In some embodiments, the mononuclear-enriched cell composition undergoes at least one washing step following incubation. In some embodiments, the mononuclear-enriched cell composition undergoes at least one washing step following incubation using PBS. In some embodiments, anti-coagulant is not added to the final washing step prior to re-suspension of the cell-preparation in the administration medium. In some embodiments, anti-coagulant is not added to the PBS used in the final washing step prior to re-suspension of the cell-preparation in the administration medium. In certain embodiments, anti-coagulant is not added to the administration medium.

In some embodiments, the cell concentration during incubating is about 5×10⁶ cells/ml.

In some embodiments, the mononuclear-enriched cell composition is suspended in an administration medium following freezing, thawing and incubating, thereby resulting in the pharmaceutical population. In some embodiments, the administration medium comprises a suitable physiological buffer. Non-limiting examples of a suitable physiological buffer are: saline solution, Phosphate Buffered Saline (PBS), Hank's Balanced Salt Solution (HBSS), and the like. In some embodiments, the administration medium comprises PBS. In some embodiments, the administration medium comprises supplements conducive to maintaining the viability of the cells. In some embodiments, the mononuclear-enriched cell composition is filtered prior to administration. In some embodiments, the mononuclear-enriched cell composition is filtered prior to administration using a filter of at least 200 μm.

In some embodiments, the mononuclear-enriched cell population is re-suspended in an administration medium such that the final volume of the resulting cell-preparation is between 100-1000 ml, possibly between 200-800 ml, typically between 300-600 ml.

In some embodiments, cell collection refers to obtaining a mononuclear-enriched cell composition. In some embodiments, washing steps performed during the production of an early apoptotic cell population are performed in a washing medium. In certain embodiments, washing steps performed up until the incubation step of the production of an early apoptotic cell population are performed in a washing medium. In some embodiments, the washing medium comprises RPMI 1640 medium supplemented with L-glutamine and Hepes. In some embodiments, the washing medium comprises RPMI 1640 medium supplemented with 2 mM L-glutamine and 10 mM Hepes.

In some embodiments, the washing medium comprises an anti-coagulant. In some embodiments, the washing medium comprises an anti-coagulant selected from the group consisting of: heparin, ACD Formula A and a combination thereof. In some embodiments, the concentration of the anti-coagulant in the washing medium is the same concentration as in the freezing medium. In some embodiments, the concentration of the anti-coagulant in the washing medium is the same concentration as in the incubation medium. In some embodiments, the anti-coagulant used in the washing medium is ACD Formula A containing heparin at a concentration of 10 U/ml.

In some embodiments, the washing medium comprises heparin. In some embodiments, the heparin in the washing medium is at a concentration of between 0.1-2.5 U/ml. In some embodiments, the heparin in the washing medium is at a concentration of between 0.1-2.5 U/ml, possibly between 0.3-0.7 U/ml, typically about 0.5 U/ml. In certain embodiments, the heparin in the washing medium is at a concentration of about 0.5 U/ml.

In some embodiments, the washing medium comprises ACD Formula A. In some embodiments, the ACD Formula A in the washing medium is at a concentration of between 1%-15% v/v. In some embodiments, the ACD Formula A in the washing medium is at a concentration of between 1%-15% v/v, possibly between 4%-7% v/v, typically about 5% v/v. In some embodiments, the ACD Formula A in the washing medium is at a concentration of about 5% v/v.

In some embodiments, the mononuclear-enriched cell composition is thawed several hours prior to the intended administration of the population to a subject. In some embodiments, the mononuclear-enriched cell composition is thawed at about 33° C.-39° C. In some embodiments, the mononuclear-enriched cell composition is thawed for about 30-240 seconds, preferably 40-180 seconds, most preferably 50-120 seconds.

In some embodiments, the mononuclear-enriched cell composition is thawed at least 10 hours prior to the intended administration of the population, alternatively at least 20, 30, 40 or 50 hours prior to the intended administration of the population. In some embodiments, the mononuclear-enriched cell composition is thawed at least 15-24 hours prior to the intended administration of the population. In some embodiments, the mononuclear-enriched cell composition is thawed at least about 24 hours prior to the intended administration of the population. In some embodiments, the mononuclear-enriched cell composition is thawed at least 20 hours prior to the intended administration of the population. In some embodiments, the mononuclear-enriched cell composition is thawed 30 hours prior to the intended administration of the population. In some embodiments, the mononuclear-enriched cell composition is thawed at least 24 hours prior to the intended administration of the population. In some embodiments, the mononuclear-enriched cell composition undergoes at least one step of washing in the washing medium before and/or after thawing.

In some embodiments, the composition further comprises methylprednisolone. At some embodiments, the concentration of methylprednisolone does not exceed 30 μg/ml.

In some embodiments, the apoptotic cells are used at a high dose. In some embodiments, the apoptotic cells are used at a high concentration. In some embodiments, human apoptotic polymorphonuclear neutrophils (PMNs) are used. In some embodiments, a group of cells, of which 50% are apoptotic cells, are used. In some embodiments, early apoptotic cells are verified by May-Giemsa-stained cytopreps. In some embodiments, viability of cells are assessed by trypan blue exclusion. In some embodiments, the apoptotic and necrotic status of the cells are confirmed by annexin V/propidium iodide staining with detection by FACS.

In some embodiments, early apoptotic cells disclosed herein comprise no necrotic cells. In some embodiments, early apoptotic cells disclosed herein comprise less than 1% necrotic cells. In some embodiments, early apoptotic cells disclosed herein comprise less than 2% necrotic cells. In some embodiments, early apoptotic cells disclosed herein comprise less than 3% necrotic cells. In some embodiments, early apoptotic cells disclosed herein comprise less than 4% necrotic cells. In some embodiments, early apoptotic cells disclosed herein comprise less than 5% necrotic cells.

In some embodiments, the apoptotic cells are prepared from cells obtained from a subject other than the subject that will receive said apoptotic cells. In some embodiments, the methods as disclosed herein comprise an additional step that is useful in overcoming rejection of allogeneic donor cells, including one or more steps described in U.S. Patent Application 20130156794, which is incorporated herein by reference in its entirety. In some embodiments, the methods comprise the step of full or partial lymphodepletion prior to administration of the apoptotic cells, which in some embodiments, are allogeneic apoptotic cells. In some embodiments, the lymphodepletion is adjusted so that it delays the host versus graft reaction for a period sufficient to allow the allogeneic apoptotic cells to control cytokine release. In some embodiments, the methods comprise the step of administering agents that delay egression of the allogeneic apoptotic T-cells from lymph nodes, such as 2-amino-2-[2-(4-octylphenyl)ethyl]propane-1,3-diol (FTY720), 5-[4-phenyl-5-(trifluoromethyl)thiophen-2-yl]-3-[3-(trifluoromethyl)pheny-1]1,2,4-oxadiazole (SEW2871), 3-(2-(−hexylphenylamino)-2-oxoethylamino)propanoic acid (W123), 2-ammonio-4-(2-chloro-4-(3-phenoxyphenylthio)phenyl)-2-(hydroxymethyl)but-yl hydrogen phosphate (KRP-203 phosphate) or other agents known in the art, may be used as part of the compositions and methods as disclosed herein to allow the use of allogeneic apoptotic cells having efficacy and lacking initiation of graft vs host disease. In another embodiment, MHC expression by the allogeneic apoptotic T-cells is silenced to reduce the rejection of the allogeneic cells.

In some embodiments, methods comprise producing a population of mononuclear apoptotic cell comprising a decreased percent of non-quiescent non-apoptotic viable cells; a suppressed cellular activation of any living non-apoptotic cells; or a reduced proliferation of any living non-apoptotic cells; or any combination thereof, said method comprising the following steps, obtaining a mononuclear-enriched cell population of peripheral blood; freezing said mononuclear-enriched cell population in a freezing medium comprising an anticoagulant; thawing said mononuclear-enriched cell population; incubating said mononuclear-enriched cell population in an apoptosis inducing incubation medium comprising methylprednisolone at a final concentration of about 10-100 μg/mL and an anticoagulant; resuspending said apoptotic cell population in an administration medium; and inactivating said mononuclear-enriched population, wherein said inactivation occurs following apoptotic induction, wherein said method produces a population of mononuclear apoptotic cell comprising a decreased percent of non-quiescent non-apoptotic cells; a suppressed cellular activation of any living non-apoptotic cells; or a reduced proliferation of any living non-apoptotic cells; or any combination thereof.

In some embodiments, the methods comprise the step of irradiating a population of apoptotic cells derived from a subject prior to administration of the population of apoptotic cells to the same subject (autologous ApoCells). In some embodiments, the methods comprise the step of irradiating apoptotic cells derived from a subject prior to administration of the population of apoptotic cells to a recipient (allogeneic ApoCells).

In some embodiments, cells are irradiated in a way that will decrease proliferation and/or activation of residual viable cells within the apoptotic cell population. In some embodiments, cells are irradiated in a way that reduces the percent of viable non-apoptotic cells in a population. In some embodiments, the percent of viable non-apoptotic cells in an inactivated early apoptotic cell population is reduced to less than 50% of the population. In some embodiments, the percent of viable non-apoptotic cells in an inactivated early apoptotic cell population is reduced to less than 40% of the population. In some embodiments, the percent of viable non-apoptotic cells in an inactivated early apoptotic cell population is reduced to less than 30% of the population. In some embodiments, the percent of viable non-apoptotic cells in an inactivated early apoptotic cell population is reduced to less than 20% of the population. In some embodiments, the percent of viable non-apoptotic cells in an inactivated early apoptotic cell population is reduced to less than 10% of the population. In some embodiments, the percent of viable non-apoptotic cells in an inactivated early apoptotic cell population is reduced to 0% of the population.

In another embodiment, the irradiated apoptotic cells preserve all their early apoptotic-, immune modulation-, stability-properties. In another embodiment, the irradiation step uses UV radiation. In another embodiment, the radiation step uses gamma radiation. In another embodiment, the apoptotic cells comprise a decreased percent of living non-apoptotic cells, comprise a preparation having a suppressed cellular activation of any living non-apoptotic cells present within the apoptotic cell preparation, or comprise a preparation having reduced proliferation of any living non-apoptotic cells present within the apoptotic cell preparation, or any combination thereof.

In some embodiments, irradiation of apoptotic cells does not increase the population of dead cells (PI+) compared with apoptotic cells not irradiated. In some embodiments, irradiation of apoptotic cells does not increase the population of dead cells (PI+) by more than about 1% compared with apoptotic cells not irradiated. In some embodiments, irradiation of apoptotic cells does not increase the population of dead cells (PI+) by more than about 2% compared with apoptotic cells not irradiated. In some embodiments, irradiation of apoptotic cells does not increase the population of dead cells (PI+) by more than about 3% compared with apoptotic cells not irradiated. In some embodiments, irradiation of apoptotic cells does not increase the population of dead cells (PI+) by more than about 4% compared with apoptotic cells not irradiated. In some embodiments, irradiation of apoptotic cells does not increase the population of dead cells (PI+) by more than about 5% compared with apoptotic cells not irradiated. In some embodiments, irradiation of apoptotic cells does not increase the population of dead cells (PI+) by more than about 6% compared with apoptotic cells not irradiated. In some embodiments, irradiation of apoptotic cells does not increase the population of dead cells (PI+) by more than about 7% compared with apoptotic cells not irradiated. In some embodiments, irradiation of apoptotic cells does not increase the population of dead cells (PI+) by more than about 8% compared with apoptotic cells not irradiated. In some embodiments, irradiation of apoptotic cells does not increase the population of dead cells (PI+) by more than about 9% compared with apoptotic cells not irradiated. In some embodiments, irradiation of apoptotic cells does not increase the population of dead cells (PI+) by more than about 10% compared with apoptotic cells not irradiated. In some embodiments, irradiation of apoptotic cells does not increase the population of dead cells (PI+) by more than about 15% compared with apoptotic cells not irradiated. In some embodiments, irradiation of apoptotic cells does not increase the population of dead cells (PI+) by more than about 20%, 25%, 30%, 35%, 40%, 45%, or 50% compared with apoptotic cells not irradiated.

In some embodiments, a cell population comprising a reduced or non-existent fraction of living non-apoptotic cells may in one embodiment provide a mononuclear early apoptotic cell population that does not have any living/viable cells. In some embodiments, a cell population comprising a reduced or non-existent fraction of living non-apoptotic cells may in one embodiment provide a mononuclear apoptotic cell population that does not elicit GVHD in a recipient.

In some embodiments, use of irradiated ApoCells removes the possible graft versus leukemia effect use of an apoptotic population (that includes a minor portion of viable cells) may cause, demonstrating that the effects result from the apoptotic cells and not from a viable proliferating population of cells with cellular activity, present within the apoptotic cell population.

In another embodiment, the methods comprise the step of irradiating apoptotic cells derived from WBCs from a donor prior to administration to a recipient. In some embodiments, cells are irradiated in a way that will avoid proliferation and/or activation of residual viable cells within the apoptotic cell population. In another embodiment, the irradiated apoptotic cells preserve all their early apoptotic-, immune modulation-, stability-properties. In another embodiment, the irradiation step uses UV radiation. In another embodiment, the radiation step uses gamma radiation. In another embodiment, the apoptotic cells comprise a decreased percent of living non-apoptotic cells, comprise a preparation having a suppressed cellular activation of any living non-apoptotic cells present within the apoptotic cell preparation, or comprise a preparation having reduced proliferation of any living non-apoptotic cells present within the apoptotic cell preparation, or any combination thereof.

In some embodiments, early apoptotic cells comprise a pooled mononuclear apoptotic cell preparation. In some embodiments, a pooled mononuclear apoptotic cell preparation comprises mononuclear cells in an early apoptotic state, wherein said pooled mononuclear apoptotic cells comprise a decreased percent of living non-apoptotic cells, a preparation having a suppressed cellular activation of any living non-apoptotic cells, or a preparation having reduced proliferation of any living non-apoptotic cells, or any combination thereof. In another embodiment, the pooled mononuclear apoptotic cells have been irradiated. In another embodiment, disclosed herein is a pooled mononuclear apoptotic cell preparation that in some embodiments, originates from the white blood cell fraction (WBC) obtained from donated blood.

In some embodiments, the apoptotic cell preparation is irradiated. In another embodiment, said irradiation comprises gamma irradiation or UV irradiation. In yet another embodiment, the irradiated preparation has a reduced number of non-apoptotic cells compared with a non-irradiated apoptotic cell preparation. In another embodiment, the irradiated preparation has a reduced number of proliferating cells compared with a non-irradiated apoptotic cell preparation. In another embodiment, the irradiated preparation has a reduced number of potentially immunologically active cells compared with a non-irradiated apoptotic cell population.

In some embodiments, pooled blood comprises 3rd party blood not matched between donor and recipient.

A skilled artisan would appreciate that the term “pooled” may encompass blood collected from multiple donors, prepared and possibly stored for later use. This combined pool of blood may then be processed to produce a pooled mononuclear apoptotic cell preparation. In another embodiment, a pooled mononuclear apoptotic cell preparation ensures that a readily available supply of mononuclear apoptotic cells is available. In another embodiment, cells are pooled just prior to the incubation step wherein apoptosis is induced. In another embodiment, cells are pooled following the incubation step at the step of resuspension. In another embodiment, cells are pooled just prior to an irradiation step. In another embodiment, cells are pooled following an irradiation step. In another embodiment, cells are pooled at any step in the methods of preparation.

In some embodiments, a pooled apoptotic cell preparation is derived from cells present in between about 2 and 25 units of blood. In another embodiment, said pooled apoptotic cell preparation is comprised of cells present in between about 2-5, 2-10, 2-15, 2-20, 5-10, 5-15, 5-20, 5-25, 10-15, 10-20, 10-25, 6-13, or 6-25 units of blood. In another embodiment, said pooled apoptotic cell preparation is comprised of cells present in about 2, 3, 4, 5, 6, 7, 8, 9, 10, 11, 12, 13, 14, 15, 16, 17, 18, 19, 20, 21, 22, 23, 24 or 25 units of blood. The number of units of blood needed is also dependent upon the efficiency of WBC recovery from blood. For example, low efficiency WBC recovery would lead to the need for additional units, while high efficiency WBC recovery would lead to fewer units needed. In some embodiments, each unit is a bag of blood. In another embodiment, a pooled apoptotic cell preparation is comprised of cells present in at least 25 units of blood, at least 50 units of blood, or at least 100 units of blood.

In some embodiments, the units of blood comprise white blood cell (WBC) fractions from blood donations. In another embodiment, the donations may be from a blood center or blood bank. In another embodiment, the donations may be from donors in a hospital gathered at the time of preparation of the pooled apoptotic cell preparation. In another embodiment, units of blood comprising WBCs from multiple donors are saved and maintained in an independent blood bank created for the purpose of compositions and methods thereof as disclosed herein. In another embodiment, a blood bank developed for the purpose of compositions and methods thereof as disclosed herein, is able to supply units of blood comprising WBC from multiple donors and comprises a leukapheresis unit.

In some embodiments, the units of pooled WBCs are not restricted by HLA matching. Therefore, the resultant pooled apoptotic cell preparation comprises cell populations not restricted by HLA matching. Accordingly, in certain embodiments a pooled mononuclear apoptotic cell preparation comprises allogeneic cells.

An advantage of a pooled mononuclear apoptotic cell preparation that is derived from pooled WBCs not restricted by HLA matching, is a readily available source of WBCs and reduced costs of obtaining WBCs.

In some embodiments, pooled blood comprises blood from multiple donors independent of HLA matching. In another embodiment, pooled blood comprises blood from multiple donors wherein HLA matching with the recipient has been taken into consideration. For example, wherein 1 HLA allele, 2 HLA alleles, 3 HLA alleles, 4 HLA alleles, 5 HLA alleles, 6 HLA alleles, or 7 HLA alleles have been matched between donors and recipient. In another embodiment, multiple donors are partially matched, for example some of the donors have been HLA matched wherein 1 HLA allele, 2 HLA alleles, 3 HLA alleles, 4 HLA alleles, 5 HLA alleles, 6 HLA alleles, or 7 HLA alleles have been matched between some of the donors and recipient. Each possibility comprises an embodiment as disclosed herein.

In certain embodiments, some viable non-apoptotic cells (apoptosis resistant) may remain following the induction of apoptosis step described below (Example 1). The presence of these viable non-apoptotic cells is, in some embodiments, is observed prior to an irradiation step. These viable non-apoptotic cells may be able to proliferate or be activated. In some embodiments, the pooled mononuclear apoptotic cell preparation derived from multiple donors may be activated against the host, activated against one another, or both.

In some embodiments, an irradiated cell preparation as disclosed herein has suppressed cellular activation and reduced proliferation compared with a non-irradiated cell preparation. In another embodiment, the irradiation comprises gamma irradiation or UV irradiation. In another embodiment, an irradiated cell preparation has a reduced number of non-apoptotic cells compared with a non-irradiated cell preparation. In another embodiment, the irradiation comprises about 15 Grey units (Gy). In another embodiment, the irradiation comprises about 20 Grey units (Gy). In another embodiment, the irradiation comprises about 25 Grey units (Gy). In another embodiment, the irradiation comprises about 30 Grey units (Gy). In another embodiment, the irradiation comprises about 35 Grey units (Gy). In another embodiment, the irradiation comprises about 40 Grey units (Gy). In another embodiment, the irradiation comprises about 45 Grey units (Gy). In another embodiment, the irradiation comprises about 50 Grey units (Gy). In another embodiment, the irradiation comprises about 55 Grey units (Gy). In another embodiment, the irradiation comprises about 60 Grey units (Gy). In another embodiment, the irradiation comprises about 65 Grey units (Gy). In another embodiment, the irradiation comprises up to 2500 Gy. In another embodiment, an irradiated pooled apoptotic cell preparation maintains the same or a similar apoptotic profile, stability and efficacy as a non-irradiated pooled apoptotic cell preparation.

In some embodiments, a pooled mononuclear apoptotic cell preparation as disclosed herein is stable for up to 24 hours. In another embodiment, a pooled mononuclear apoptotic cell preparation is stable for at least 24 hours. In another embodiment, a pooled mononuclear apoptotic cell preparation is stable for more than 24 hours. In yet another embodiment, a pooled mononuclear apoptotic cell preparation as disclosed herein is stable for up to 36 hours. In still another embodiment, a pooled mononuclear apoptotic cell preparation is stable for at least 36 hours. In a further embodiment, a pooled mononuclear apoptotic cell preparation is stable for more than 36 hours. In another embodiment, a pooled mononuclear apoptotic cell preparation as disclosed herein is stable for up to 48 hours. In another embodiment, a pooled mononuclear apoptotic cell preparation is stable for at least 48 hours. In another embodiment, a pooled mononuclear apoptotic cell preparation is stable for more than 48 hours.

In some embodiments, methods of producing the pooled cell preparation comprising an irradiation step preserves the early apoptotic, immune modulation, and stability properties observed in an apoptotic preparation derived from a single match donor wherein the cell preparation may not include an irradiation step. In another embodiment, a pooled mononuclear apoptotic cell preparation as disclosed herein does not elicit a graft versus host disease (GVHD) response.

Irradiation of the cell preparation is considered safe in the art. Irradiation procedures are currently performed on a routine basis to donated blood to prevent reactions to WBC.

In another embodiment, the percent of apoptotic cells in a pooled mononuclear apoptotic cell preparation as disclosed herein is close to 100%, thereby reducing the fraction of living non-apoptotic cells in the cell preparation. In some embodiments, the percent of apoptotic cells is at least 40%. In another embodiment, the percent of apoptotic cells is at least 50%. In yet another embodiment, the percent of apoptotic cells is at least 60%. In still another embodiment, the percent of apoptotic cells is at least 70%. In a further embodiment, the percent of apoptotic cells is at least 80%. In another embodiment, the percent of apoptotic cells is at least 90%. In yet another embodiment, the percent of apoptotic cells is at least 99%. Accordingly, a cell preparation comprising a reduced or non-existent fraction of living non-apoptotic cells may in one embodiment provide a pooled mononuclear apoptotic cell preparation that does not elicit GVHD in a recipient. Each possibility represents an embodiment as disclosed herein.

Alternatively, in another embodiment, the percentage of living non-apoptotic WBC is reduced by specifically removing the living cell population, for example by targeted precipitation. In another embodiment, the percent of living non-apoptotic cells may be reduced using magnetic beads that bind to phosphatidylserine. In another embodiment, the percent of living non-apoptotic cells may be reduced using magnetic beads that bind a marker on the cell surface of non-apoptotic cells but not apoptotic cells. In another embodiment, the apoptotic cells may be selected for further preparation using magnetic beads that bind to a marker on the cell surface of apoptotic cells but not non-apoptotic cells. In yet another embodiment, the percentage of living non-apoptotic WBC is reduced by the use of ultrasound.

In one embodiment the apoptotic cells are from pooled third-party donors.

In some embodiments, a pooled cell preparation comprises at least one cell type selected from the group consisting of: lymphocytes, monocytes and natural killer cells. In another embodiment, a pooled cell preparation comprises an enriched population of mononuclear cells. In some embodiments, a pooled mononuclear is a mononuclear enriched cell preparation comprises cell types selected from the group consisting of: lymphocytes, monocytes and natural killer cells. In another embodiment, the mononuclear enriched cell preparation comprises no more than 15%, alternatively no more than 10%, typically no more than 5% polymorphonuclear leukocytes, also known as granulocytes (i.e., neutrophils, basophils and eosinophils). In another embodiment, a pooled mononuclear cell preparation is devoid of granulocytes.

In another embodiment, the pooled mononuclear enriched cell preparation comprises no more than 15%, alternatively no more than 10%, typically no more than 5% CD15^{high} expressing cells. In some embodiments, a pooled apoptotic cell preparation comprises less than 15% CD15 high expressing cells.

In some embodiments, the pooled mononuclear enriched cell preparation disclosed herein comprises at least 80% mononuclear cells, at least 85% mononuclear cells, alternatively at least 90% mononuclear cells, or at least 95% mononuclear cells, wherein each possibility is a separate embodiment disclosed herein. According to some embodiments, the pooled mononuclear enriched cell preparation disclosed herein comprises at least 85% mononuclear cells.

In another embodiment, any pooled cell preparation that has a final pooled percent of mononuclear cells of at least 80% is considered a pooled mononuclear enriched cell preparation as disclosed herein. Thus, pooling cell preparations having increased polymorphonuclear cells (PMN) with cell preparations having high mononuclear cells with a resultant “pool” of at least 80% mononuclear cells comprises a preparation as disclosed herein. According to some embodiments, mononuclear cells comprise lymphocytes and monocytes.

A skilled artisan would appreciate that the term “mononuclear cells” may encompass leukocytes having a one lobed nucleus. In another embodiment, a pooled apoptotic cell preparation as disclosed herein comprises less than 5% polymorphonuclear leukocytes.

Surprisingly, the apoptotic cells reduce production of cytokines associated with the cytokine storm including but not limited to IL-6, and interferon-gamma (IFN-γ), alone or in combination. In one embodiment, the apoptotic cells affect cytokine expression levels in macrophages. In another embodiment, the apoptotic cells reduce cytokine expression levels in macrophages. In one embodiment, the apoptotic cells suppress cytokine expression levels in macrophages. In one embodiment, the apoptotic cells inhibit cytokine expression levels in macrophages.

In another embodiment, the effect of apoptotic cells on cytokine expression levels in macrophages, DCs, or a combination thereof, results in reduction of CRS. In another embodiment, the effect of apoptotic cells on cytokine expression levels in macrophages, DCs, or a combination thereof, results in reduction of severe CRS. In another embodiment, the effect of apoptotic cells on cytokine expression levels in macrophages, DCs, or a combination thereof, results in suppression of CRS. In another embodiment, the effect of apoptotic cells on cytokine expression levels in macrophages, DCs, or a combination thereof, results in suppression of severe CRS. In another embodiment, the effect of apoptotic cells on cytokine expression levels in macrophages, DCs, or a combination thereof, results in inhibition of CRS. In another embodiment, the effect of apoptotic cells on cytokine expression levels in macrophages, DCs, or a combination thereof, results in inhibition of severe CRS. In another embodiment, the effect of apoptotic cells on cytokine expression levels in macrophages, DCs, or a combination thereof, results in prevention of CRS. In another embodiment, the effect of apoptotic cells on cytokine expression levels in macrophages, DCs, or a combination thereof, results in prevention of severe CRS.

In another embodiment, the apoptotic cells trigger death of T-cells, but not via changes in cytokine expression levels.

In another embodiment, early apoptotic cells antagonize the priming of macrophages and dendritic cells to secrete cytokines that would otherwise amplify the cytokine storm. In another embodiment, early apoptotic cells increase Tregs which suppress the inflammatory response and/or prevent excess release of cytokines.

In some embodiments, administration of apoptotic cells inhibits one or more pro-inflammatory cytokines. In some embodiments, the pro-inflammatory cytokine comprises IL-1beta, IL-6, TNF-alpha, or IFN-gamma, or any combination thereof. In some embodiments, inhibition of one or more pro-inflammatory cytokines comprises downregulation of pr0-inflammatory cytokines, wherein a reduced amount of one or more pro-inflammatory cytokines is secreted.

In another embodiment, administration of apoptotic cells promotes the secretion of one or more anti-inflammatory cytokines. In some embodiments, the anti-inflammatory cytokine comprises TGF-beta, IL10, or PGE2, or any combination thereof.

In some embodiments, administration of apoptotic cells inhibits one or more pro-inflammatory cytokine and inhibits on or more anti-inflammatory cytokine. In some embodiments, inhibition of one or more pro-inflammatory cytokine and one or more anti-inflammatory cytokine comprises downregulation of the one or more pro-inflammatory cytokines followed by downregulation of one or more anti-inflammatory cytokine, wherein a reduced amount of the one or more pro-inflammatory cytokines and the one or move anti-inflammatory cytokine is secreted. A skilled artisan would appreciate that apoptotic cells may therefore have a beneficial effect on aberrant innate immune response, with downregulation of both anti- and pro-inflammatory cytokines. In some embodiments, this beneficial effect may follow recognition of PAMPs and DAMPs by components of the innate immune system.

In another embodiment, administration of apoptotic cells inhibits dendritic cell maturation following exposure to TLR ligands. In another embodiment, administration of apoptotic cells creates potentially tolerogenic dendritic cells, which in some embodiments, are capable of migration, and in some embodiments, the migration is due to CCR7. In another embodiment, administration of apoptotic cells elicits various signaling events which in one embodiment is TAM receptor signaling (Tyro3, Axl and Mer) which in some embodiments, inhibits inflammation in antigen-presenting cells.

In some embodiments, Tyro-3, Axl, and Mer constitute the TAM family of receptor tyrosine kinases (RTKs) characterized by a conserved sequence within the kinase domain and adhesion molecule-like extracellular domains. In another embodiment, administration of apoptotic cells activates signaling through MerTK. In another embodiment, administration of apoptotic cells activates the phosphatidylinositol 3-kinase (PI3K)/AKT pathway, which in some embodiments, negatively regulates NF-κB. In another embodiment, administration of apoptotic cells negatively regulates the inflammasome which in one embodiment leads to inhibition of pro-inflammatory cytokine secretion, DC maturation, or a combination thereof. In another embodiment, administration of apoptotic cells upregulates expression of anti-inflammatory genes such as Nr4a, Thbs1, or a combination thereof. In another embodiment, administration of apoptotic cells induces a high level of AMP which in some embodiments, is accumulated in a Pannexin1-dependent manner. In another embodiment, administration of apoptotic cells suppresses inflammation.

### Apoptotic Cell Supernatants (ApoSup and ApoSup Mon)

In some embodiments, compositions for use in the methods and treatments as disclosed herein include an apoptotic cell supernatant as disclosed herein.

In some embodiments, the apoptotic cell supernatant is obtained by a method comprising the steps of a) providing apoptotic cells, b) culturing the apoptotic cells of step a), and c) separating the supernatant from the cells.

In some embodiments, early apoptotic cells for use making an apoptotic cell supernatant as disclosed herein are autologous with a subject undergoing therapy. In another embodiment, early apoptotic cells for use in making an apoptotic cell supernatant disclosed herein are allogeneic with a subject undergoing therapy.

The “apoptotic cells” from which the apoptotic cell supernatant is obtained may be cells chosen from any cell type of a subject, or any commercially available cell line, subjected to a method of inducing apoptosis known to the person skilled in the art. The method of inducing apoptosis may be hypoxia, ozone, heat, radiation, chemicals, osmotic pressure, pH shift, X-ray irradiation, gamma-ray irradiation, UV irradiation, serum deprivation, corticoids or combinations thereof, or any other method described herein or known in the art. In another embodiment, the method of inducing apoptosis produces apoptotic cells in an early apoptotic state.

In some embodiments, the apoptotic cells are leukocytes.

In an embodiment, said apoptotic leukocytes are derived from peripheral blood mononuclear cells (PBMC). In another embodiment, said leukocytes are from pooled third-party donors. In another embodiment, said leukocytes are allogeneic.

According to some embodiments, the apoptotic cells are provided by selecting non-adherent leukocytes and submitting them to apoptosis induction, followed by a cell culture step in culture medium. “Leukocytes” used to make the apoptotic cell-phagocyte supernatant may be derived from any lineage, or sub-lineage, of nucleated cells of the immune system and/or hematopoietic system, including but not limited to dendritic cells, macrophages, masT-cells, basophils, hematopoietic stem cells, bone marrow cells, natural killer cells, and the like. The leukocytes may be derived or obtained in any of various suitable ways, from any of various suitable anatomical compartments, according to any of various commonly practiced methods, depending on the application and purpose, desired leukocyte lineage, etc. In some embodiments, the source leukocytes are primary leukocytes. In another embodiment, the source leukocytes are primary peripheral blood leukocytes.

Primary lymphocytes and monocytes may be conveniently derived from peripheral blood. Peripheral blood leukocytes include 70-95 percent lymphocytes, and 5-25 percent monocytes.

Methods for obtaining specific types of source leukocytes from blood are routinely practiced. Obtaining source lymphocytes and/or monocytes can be achieved, for example, by harvesting blood in the presence of an anticoagulant, such as heparin or citrate. The harvested blood is then centrifuged over a Ficoll cushion to isolate lymphocytes and monocytes at the gradient interface, and neutrophils and erythrocytes in the pellet.

Leukocytes may be separated from each other via standard immunomagnetic selection or immunofluorescent flow cytometry techniques according to their specific surface markers, or via centrifugal elutriation. For example, monocytes can be selected as the CD14+ fraction, T-lymphocytes can be selected as CD3+ fraction, B-lymphocytes can be selected as the CD19+ fraction, macrophages as the CD206+ fraction.

Lymphocytes and monocytes may be isolated from each other by subjecting these cells to substrate-adherent conditions, such as by static culture in a tissue culture-treated culturing recipient, which results in selective adherence of the monocytes, but not of the lymphocytes, to the cell-adherent substrate.

Leukocytes may also be obtained from peripheral blood mononuclear cells (PBMCs), which may be isolated as described herein.

One of ordinary skill in the art will possess the necessary expertise to suitably culture primary leukocytes so as to generate desired quantities of cultured source leukocytes as disclosed herein, and ample guidance for practicing such culturing methods is available in the literature of the art.

One of ordinary skill in the art will further possess the necessary expertise to establish, purchase, or otherwise obtain suitable established leukocyte cell lines from which to derive the apoptotic leukocytes. Suitable leukocyte cell lines may be obtained from commercial suppliers, such as the American Tissue Type Collection (ATCC). It will be evident to the person skilled in the art that source leukocytes should not be obtained via a technique which will significantly interfere with their capacity to produce the apoptotic leukocytes.

In another embodiment, the apoptotic cells may be apoptotic lymphocytes. Apoptosis of lymphocytes, such as primary lymphocytes, may be induced by treating the primary lymphocytes with serum deprivation, a corticosteroid, or irradiation. In another embodiment, inducing apoptosis of primary lymphocytes via treatment with a corticosteroid is effected by treating the primary lymphocytes with dexamethasone. In another embodiment, with dexamethasone at a concentration of about 1 micromolar. In another embodiment, inducing apoptosis of primary lymphocytes via irradiation is effected by treating the primary lymphocytes with gamma-irradiation. In another embodiment, with a dosage of about 66 rad. Such treatment results in the generation of apoptotic lymphocytes suitable for the co-culture step with phagocytes.

In a further embodiment, early apoptotic cells may be apoptotic monocytes, such as primary monocytes. To generate apoptotic monocytes the monocytes are subjected to in vitro conditions of substrate/surface-adherence under conditions of serum deprivation. Such treatment results in the generation of non-pro-inflammatory apoptotic monocytes suitable for the co-culture step with phagocytes.

In other embodiments, the apoptotic cells may be any apoptotic cells described herein, including allogeneic apoptotic cells, third party apoptotic cells, and pools of apoptotic cells.

In other embodiments, the apoptotic cell supernatant may be obtained through the co-culture of apoptotic cells with other cells.

Thus, in some embodiments, the apoptotic cell supernatant is an apoptotic cell supernatant obtained by a method comprising the steps of a) providing apoptotic cells, b) providing other cells, c) optionally washing the cells from step a) and b), d) co-culturing the cells of step a) and b), and optionally e) separating the supernatant from the cells.

In some embodiments, the other cells co-cultured with the apoptotic cells are white blood cells.

Thus, in some embodiments, the apoptotic cell supernatant is an apoptotic cell-white blood cell supernatant obtained by a method comprising the steps of a) providing apoptotic cells, b) providing white blood cells, c) optionally washing the cells from step a) and b), d) co-culturing the cells of step a) and b), and optionally e) separating the supernatant from the cells.

In some embodiments, the white blood cells may be phagocytes, such as macrophages, monocytes or dendritic cells.

In some embodiments, the white blood cells may be B cells, T-cells, or natural killer (NK cells).

Thus, in some embodiments, compositions for use in the methods and treatments as disclosed herein include apoptotic cell-phagocyte supernatants as described in WO 2014/106666, which is incorporated by reference herein in its entirety. In another embodiment, early apoptotic cell-phagocyte supernatants for use in compositions and methods as disclosed herein are produced in any way that is known in the art.

In some embodiments, the apoptotic cell-phagocyte supernatant is obtained from a co-culture of phagocytes with apoptotic cells,

In some embodiments, the apoptotic cell-phagocyte supernatant is obtained by a method comprising the steps of a) providing phagocytes, b) providing apoptotic cells, c) optionally washing the cells from step a) and b), d) co-culturing the cells of step a) and b), and optionally e) separating the supernatant from the cells.

The term “phagocytes” denotes cells that protect the body by ingesting (phagocytosing) harmful foreign particles, bacteria, and dead or dying cells. Phagocytes include for example cells called neutrophils, monocytes, macrophages, dendritic cells, and mast T-cells, preferentially dendritic cells and monocytes/macrophages. The phagocytes may be dendritic cells (CD4+ HLA-DR+ Lineage− BDCA1/BDCA3+), macrophages (CD14+ CD206+ HLA-DR+), or derived from monocytes (CD14+). Techniques to distinguish these different phagocytes are known to the person skilled in the art.

In an embodiment, monocytes are obtained by a plastic adherence step. Said monocytes can be distinguished from B and T-cells with the marker CD14+, whereas unwanted B cells express CD19+ and T-cells CD3+. After Macrophage Colony Stimulating Factor (M-CSF) induced maturation the obtained macrophages are in some embodiments, positive for the markers CD14+, CD206+, HLA-DR+.

In an embodiment, said phagocytes are derived from peripheral blood mononuclear cells (PBMC).

Phagocytes may be provided by any method known in the art for obtaining phagocytes. In some embodiments, phagocytes such as macrophages or dendritic cells can be directly isolated from a subject or be derived from precursor cells by a maturation step.

In some embodiments, macrophages may be directly isolated from the peritoneum cavity of a subject and cultured in complete RRPMI medium. Macrophages can also be isolated from the spleen.

Phagocytes are also obtainable from peripheral blood monocytes. In said example, monocytes when cultured differentiate into monocyte-derived macrophages upon addition of, without limitation to, macrophage colony stimulating factor (M-CSF) to the cell culture media.

For example, phagocytes may be derived from peripheral blood mononuclear cells (PBMC). For example, PBMC may be isolated from cytapheresis bag from an individual through Ficoll gradient centrifugation, plated in a cell-adherence step for 90 min in complete RPMI culture medium (10% FBS, 1% Penicillin/Streptomycin). Non-adherent T-cells are removed by a plastic adherence step, and adherent T-cells cultured in complete RPMI milieu supplemented with recombinant human M-CSF. After the culture period, monocyte-derived macrophages are obtained.

Phagocytes can be selected by a cell-adherence step. Said “cell adherence step” means that phagocytes or cells which can mature into phagocytes are selected via culturing conditions allowing the adhesion of the cultured cells to a surface, a cell adherent surface (e.g. a tissue culture dish, a matrix, a sac or bag with the appropriate type of nylon or plastic). A skilled artisan would appreciate that the term “Cell adherent surfaces” may encompass hydrophilic and negatively charged, and may be obtained in any of various ways known in the art, In another embodiment by modifying a polystyrene surface using, for example, corona discharge, or gas-plasma. These processes generate highly energetic oxygen ions which graft onto the surface polystyrene chains so that the surface becomes hydrophilic and negatively charged. Culture recipients designed for facilitating cell-adherence thereto are available from various commercial suppliers (e.g. Corning, Perkin-Elmer, Fisher Scientific, Evergreen Scientific, Nunc, etc.).

B cells, T-cells and NK cells may be provided by any method known in the art for obtaining such cells. In some embodiments, B cells, T-cells or NK cells can be directly isolated from a subject or be derived from precursor cells by a maturation step. In another embodiment, the B, T or NK cells can be from a B, T or NK cell line. One of ordinary skill in the art will possess the necessary expertise to establish, purchase, or otherwise obtain suitable established B cells, T-cells and NK cell lines. Suitable cell lines may be obtained from commercial suppliers, such as the American Tissue Type Collection (ATCC).

In an embodiment, said apoptotic cells and said white blood cells, such as the phagocytes, B, T or NK cells, are cultured individually prior to the co-culture step d).

The cell maturation of phagocytes takes place during cell culture, for example due to addition of maturation factors to the media. In one embodiment said maturation factor is M-CSF, which may be used for example to obtain monocyte-derived macrophages.

The culture step used for maturation or selection of phagocytes might take several hours to several days. In another embodiment said pre-mature phagocytes are cultured for 2, 4, 6, 8, 10, 12, 14, 16, 18, 20, 22, 24, 26, 28, 30, 32, 34, 36, 38, 40, 42, 44, 46, 48, 50, 52, 54, 56, 58 hours in an appropriate culture medium.

The culture medium for phagocytes is known to the person skilled in the art and can be for example, without limitation, RPMI, DMEM, X-vivo and Ultraculture milieus.

In an embodiment, co-culture of apoptotic cells and phagocytes takes place in a physiological solution.

Prior to this “co-culture”, the cells may be submitted to a washing step. In some embodiments, the white blood cells (e.g. the phagocytes) and the apoptotic cells are washed before the co-culture step. In another embodiment, the cells are washed with PBS.

During said co-culture the white blood cells (e.g. the phagocytes such as macrophages, monocytes, or phagocytes, or the B, T or NK cells) and the apoptotic cells may be mixed in a ratio of 10:1, 9:1; 8:1, 7:1, 6:1, 5:1, 4:1, 3:1, 2:1, or 1:1, or in a ratio of (white blood cells:apoptotic cells) 1:2, 1:3, 1:4, 1:5, 1:6, 1:7, 1:8, 1:9, or 1:10. In one example, the ratio of white blood cells to apoptotic cells is 1:5.

The co-culture of the cells might be for several hours to several days. In some embodiments, said apoptotic cells are cultured for 2, 4, 6, 8, 10, 12, 14, 16, 18, 20, 22, 24, 26, 28, 30, 32, 34, 36, 38, 40, 42, 44, 46, 48, 50, 52 hours. A person skilled in the art can evaluate the optimal time for co-culture by measuring the presence of anti-inflammatory compounds, the viable amount of white blood cells and the number of apoptotic cells which have not been eliminated so far. elimination of apoptotic cells by phagocytes is observable with light microscopy due to the disappearance of apoptotic cells.

In some embodiments, the culture of apoptotic cells, such as the co-culture with culture with white blood cells (e.g. phagocytes such as macrophages, monocytes, or phagocytes, or the B, T or NK cells), takes place in culture medium and/or in a physiological solution compatible with administration e.g. injection to a subject.

A skilled artisan would appreciate that a “physiological solution” may encompass a solution which does not lead to the death of white blood cells within the culture time. In some embodiments, the physiological solution does not lead to death over 2, 4, 6, 8, 10, 12, 14, 16, 18, 20, 22, 24, 26, 28, 30, 32, 34, 36, 38, 40, 42, 44, 46, 48, 50, 52 hours. In other embodiment, 48 hours, or 30 hours.

In some embodiments, the white blood cells (e.g. phagocytes such as macrophages, monocytes, or phagocytes, or the B, T or NK cells) and the apoptotic cells are incubated in the physiological solution for at least 30 min. This time of culture allows phagocytosis initiation and secretion of cytokines and other beneficial substances.

In an embodiment, such a physiological solution does not inhibit apoptotic leukocyte elimination by leukocyte-derived macrophages.

At the end of the culture or the co-culture step, the supernatant is optionally separated from the cultured apoptotic cells or the co-cultured cells. Techniques to separate the supernatant from the cells are known in the art. For example, the supernatant can be collected and/or filtered and/or centrifuged to eliminate cells and debris. For example, said supernatant may be centrifuged at 3000 rpm for 15 minutes at room temperature to separate it from the cells.

The supernatant may be “inactivated” prior to use, for example by irradiation. Therefore, the method for preparing the apoptotic cell supernatant may comprise an optional additional irradiation step f). Said “irradiation” step can be considered as a disinfection method that uses X-ray irradiation (25-45 Gy) at sufficiently rate to kill microorganisms, as routinely performed to inactivate blood products.

Irradiation of the supernatant is considered safe in the art. Irradiation procedures are currently performed on a routine basis to donated blood to prevent reactions to WBC.

In an embodiment, the apoptotic cell supernatant is formulated into a pharmaceutical composition suitable for administration to a subject, as described in detail herein.

In some embodiments, the final product is stored at +4° C. In another embodiment, the final product is for use in the next 48 hours.

In some embodiments, the apoptotic cell supernatant, such as an apoptotic cell-phagocyte supernatant, or pharmaceutical composition comprising the supernatant, may be lyophilized, for example for storage at −80° C.

In one specific embodiment, as described in Example 1 of WO 2014/106666, an apoptotic cell-phagocyte supernatant may be made using thymic cells as apoptotic cells. After isolation, thymic cells are irradiated (e.g., with a 35 X-Gray irradiation) and cultured in complete DMEM culture medium for, for example, 6 hours to allow apoptosis to occur. In parallel, macrophages are isolated from the peritoneum cavity, washed and cultured in complete RPMI (10% FBS, Peni-Strepto, EAA, Hepes, NaP and 2-MercaptoEthanol). Macrophages and apoptotic cells are then washed and co-cultured for another 48 hour period in phenol-free X-vivo medium at a 1/5 macrophage/apoptotic cell ratio. Then, supernatant is collected, centrifuged to eliminate debris and may be frozen or lyophilized for conservation. Macrophage enrichment may be confirmed using positive staining for F4/80 by FACS. Apoptosis may be confirmed by FACS using positive staining for Annexin-V and 7AAD exclusion.

In an embodiment, the apoptotic cell supernatant is enriched in TGF-β levels both in active and latent forms of TGF-β, compared to supernatants obtained from either macrophages or apoptotic cells cultured separately. In an embodiment, IL-10 levels are also increased compared to macrophages cultured alone and dramatically increased compared to apoptotic cells cultured alone. In another embodiment, inflammatory cytokines such as IL-6 are not detectable and IL-1 β and TNF are undetectable or at very low levels.

In an embodiment, the apoptotic cell supernatant, when compared to supernatants from macrophages cultured alone or from apoptotic cells cultured alone, has increased levels of IL-1ra, TIMP-1, CXCL1/KC and CCL2/JE/MCP1, which might be implicated in a tolerogenic role of the supernatant to control inflammation, in addition to TGF-β and IL-10.

In another specific embodiment, as described in Example 3 of WO 2014/106666, human apoptotic cell-phagocyte supernatant may be made from the co-culture of macrophages derived from peripheral blood mononuclear cells (PBMC) cultured with apoptotic PBMC. Thus, PBMC are isolated from cytapheresis bag from a healthy volunteer through, for example, Ficoll gradient centrifugation. Then PBMC are plated for 90 min in complete RPMI culture medium (10% FBS, 1% Penicillin/Streptomycin). Then, non-adherent T-cells are removed and rendered apoptotic using, for example, a 35 Gy dose of X-ray irradiation and cultured in complete RPMI milieu for 4 days (including cell wash after the first 48 hrs of culture), in order to allow apoptosis to occur. In parallel, adherent T-cells are cultured in complete RPM′ milieu supplemented with 50 μg/mL of recombinant human M-CSF for 4 days including cell wash after the first 48 hrs. At the end of the 4-day culture period, monocyte-derived macrophages and apoptotic cells are washed and cultured together in X-vivo medium for again 48 hours at a one macrophage to 5 apoptotic cell ratio. Then supernatant from the latter culture is collected, centrifuged to eliminate cells and debris, and may be frozen or lyophilized for conservation and subsequent use.

In an embodiment, as described in WO 2014/106666, human apoptotic cell-phagocyte supernatant may be obtained in 6 days from peripheral blood mononuclear cells (PBMC). Four days to obtain PBMC-derived macrophages using M-CSF addition in the culture, and 2 more days for the co-culture of PBMC-derived macrophages with apoptotic cells, corresponding to the non-adherent PBMC isolated at day 0.

In an embodiment, as described in WO 2014/106666, a standardized human apoptotic cell-phagocyte supernatant may be obtained independently of the donor or the source of PBMC (cytapheresis or buffy coat). The plastic-adherence step is sufficient to obtain a significant starting population of enriched monocytes (20 to 93% of CD14+ cells after adherence on plastic culture dish). In addition, such adherent T-cells demonstrate a very low presence of B and T-cells (1.0% of CD19+ B cells and 12.8% of CD3+ T-cells). After 4 days of culture of adherent T-cells in the presence of M-CSF, the proportion of monocytes derived-macrophages is significantly increased from 0.1% to 77.7% of CD14+CD206+HLA-DR+ macrophages. At that time, monocyte-derived macrophages may be co-cultured with apoptotic non-adherent PBMC (47.6% apoptotic as shown by annexin V staining and 7AAD exclusion) to produce the apoptotic cell-phagocyte supernatant during 48 hours.

In an embodiment, the collected apoptotic cell-phagocyte supernatant, contains significantly more latent TGF than in the culture supernatant of monocyte-derived macrophages alone or monocyte-derived macrophages treated in inflammatory conditions (+LPS), and only contains trace or low level of inflammatory cytokines such as IL-10 or TNF.

In some embodiments, the composition comprising the apoptotic cell supernatant further comprises an anti-coagulant. In some embodiments, the anti-coagulant is selected from the group consisting of: heparin, acid citrate dextrose (ACD) Formula A and a combination thereof.

In another embodiment, an anti-coagulant is added during the process of manufacturing apoptotic cells. In another embodiment, the anti-coagulant added is selected from the group comprising ACD and heparin, or any combination thereof. In another embodiment, ACD is at a concentration of 1%. In another embodiment, ACD is at a concentration of 2%. In another embodiment, ACD is at a concentration of 3%. In another embodiment, ACD is at a concentration of 4%. In another embodiment, ACD is at a concentration of 5%. In another embodiment, ACD is at a concentration of 6%. In another embodiment, ACD is at a concentration of 7%. In another embodiment, ACD is at a concentration of 8%. In another embodiment, ACD is at a concentration of 9%. In another embodiment, ACD is at a concentration of 10%. In another embodiment, ACD is at a concentration of between about 1-10%. In another embodiment, ACD is at a concentration of between about 2-8%. In another embodiment, ACD is at a concentration of between about 3-7%. In another embodiment, ACD is at a concentration of between about 1-5%. In another embodiment, ACD is at a concentration of between about 5-10%. In another embodiment, heparin is at a final concentration of 0.5 U/ml. In another embodiment, heparin is at a final concentration of about 0.1 U/ml-1.0 U/ml. In another embodiment, heparin is at a final concentration of about 0.2 U/ml-0.9 U/ml. In another embodiment, heparin is at a final concentration of about 0.3 U/ml-0.7 U/ml. In another embodiment, heparin is at a final concentration of about 0.1 U/ml-0.5 U/ml. In another embodiment, heparin is at a final concentration of about 0.5 U/ml-1.0 U/ml. In another embodiment, heparin is at a final concentration of about 0.01 U/ml-1.0 U/ml. In another embodiment, heparin is at a final concentration of 0.1 U/ml. In another embodiment, heparin is at a final concentration of 0.2 U/ml. In another embodiment, heparin is at a final concentration of 0.3 U/ml. In another embodiment, heparin is at a final concentration of 0.4 U/ml. In another embodiment, heparin is at a final concentration of 0.5 U/ml. In another embodiment, heparin is at a final concentration of 0.6 U/ml. In another embodiment, heparin is at a final concentration of 0.7 U/ml. In another embodiment, heparin is at a final concentration of 0.8 U/ml. In another embodiment, heparin is at a final concentration of 0.9 U/ml. In another embodiment, heparin is at a final concentration of 1.0 U/ml. In another embodiment, ACD is at a concentration of 5% and heparin is at a final concentration of 0.5 U/ml.

In some embodiments, the composition comprising the apoptotic cell supernatant further comprises methylprednisolone. At some embodiments, the concentration of methylprednisolone does not exceed 30 μg/ml.

In some embodiments, the composition may be used at a total dose or aliquot of apoptotic cell supernatant derived from the co-culture of about 14×10⁹ of CD45+ cells obtained by cytapheresis equivalent to about 200 million of cells per kilogram of body weight (for a 70 kg subject). In an embodiment, such a total dose is administered as unit doses of supernatant derived from about 100 million cells per kilogram body weight, and/or is administered as unit doses at weekly intervals, In another embodiment both of which. Suitable total doses according to this embodiment include total doses of supernatant derived from about 10 million to about 4 billion cells per kilogram body weight. In another embodiment, the supernatant is derived from about 40 million to about 1 billion cells per kilogram body weight. In yet another embodiment the supernatant is derived from about 80 million to about 500 million cells per kilogram body weight. In still another embodiment, the supernatant is derived from about 160 million to about 250 million cells per kilogram body weight. Suitable unit doses according to this embodiment include unit doses of supernatant derived from about 4 million to about 400 million cells per kilogram body weight. In another embodiment, the supernatant is derived from about 8 million to about 200 million cells per kilogram body weight. In another embodiment, the supernatant is derived from about 16 million to about 100 million cells per kilogram body weight. In yet another embodiment, the supernatant is derived from about 32 million to about 50 million cells per kilogram body weight.

Surprisingly, the apoptotic cell supernatants, such as apoptotic cell-phagocyte supernatants, reduces production of cytokines associated with the cytokine storm such as IL-6. Another cytokine, IL-2, is not involved in cytokine release syndrome although is secreted by DCs and macrophages in small quantities.

In some embodiments, the apoptotic cell supernatants, such as apoptotic cell-phagocyte supernatants, affect cytokine expression levels in macrophages and DCs, but do not affect cytokine expression levels in the T-cells themselves.

In another embodiment, the apoptotic cell supernatants trigger death of T-cells, but not via changes in cytokine expression levels.

In another embodiment, early apoptotic cell supernatants, such as apoptotic cell-phagocyte supernatants antagonize the priming of macrophages and dendritic cells to secrete cytokines that would otherwise amplify the cytokine storm. In another embodiment, early apoptotic cell supernatants increase Tregs which suppress the inflammatory response and/or prevent excess release of cytokines.

In some embodiments, administration of apoptotic cell supernatants, such as apoptotic cell-phagocyte supernatants, inhibits one or more pro-inflammatory cytokines. In some embodiments, the pro-inflammatory cytokine comprises IL-1beta, IL-6, TNF-alpha, or IFN-gamma, or any combination thereof. In another embodiment, administration of apoptotic cell supernatants promotes the secretion of one or more anti-inflammatory cytokines. In some embodiments, the anti-inflammatory cytokine comprises TGF-beta, IL10, or PGE2, or any combination thereof.

In another embodiment, administration of apoptotic cell supernatants, such as apoptotic cell-phagocyte supernatants, inhibits dendritic cell maturation following exposure to TLR ligands. In another embodiment, administration of apoptotic cell supernatants creates potentially tolerogenic dendritic cells, which in some embodiments, are capable of migration, and in some embodiments, the migration is due to CCR7. In another embodiment, administration of apoptotic cell supernatants elicits various signaling events which in one embodiment is TAM receptor signaling (Tyro3, Axl and Mer) which in some embodiments, inhibits inflammation in antigen-presenting cells. In some embodiments, Tyro-3, Axl, and Mer constitute the TAM family of receptor tyrosine kinases (RTKs) characterized by a conserved sequence within the kinase domain and adhesion molecule-like extracellular domains. In another embodiment, administration of apoptotic cell supernatants activates signaling through MerTK. In another embodiment, administration of apoptotic cell supernatants activates the phosphatidylinositol 3-kinase (PI3K)/AKT pathway, which in some embodiments, negatively regulates NF-κB. In another embodiment, administration of apoptotic cell supernatants negatively regulates the inflammasome which in one embodiment leads to inhibition of pro-inflammatory cytokine secretion, DC maturation, or a combination thereof. In another embodiment, administration of apoptotic cell supernatants upregulates expression of anti-inflammatory genes such as Nr4a, Thbs1, or a combination thereof. In another embodiment, administration of apoptotic cell supernatants induces a high level of AMP which in some embodiments, is accumulated in a Pannexin1-dependent manner. In another embodiment, administration of apoptotic cell supernatants suppresses inflammation.

### Compositions

As used herein, the terms “composition” and pharmaceutical composition” may in some embodiments, be used interchangeably having all the same qualities and meanings. In some embodiments, disclosed herein is a pharmaceutical composition for the treatment of a condition or disease as described herein.

In some embodiments, disclosed herein are pharmaceutical compositions for reducing or inhibiting the incidence of CRS or a cytokine storm. In another embodiment, disclosed herein are compositions treating COVID-19 in a subject. In another embodiment, compositions for treating COVID-19 in a subject, further comprise reducing or inhibiting the incidence of CRS or a cytokine storm.

In another embodiment, a pharmaceutical composition comprises an early apoptotic cell population. In another embodiment, a pharmaceutical composition comprises an apoptotic supernatant.

In still another embodiment, a pharmaceutical composition for the treatment of COVID-19, as described herein, comprises an effective amount of an early apoptotic cell mononuclear-enriched population, as described herein, in a pharmaceutically acceptable excipient. In still another embodiment, a pharmaceutical composition for the treatment of COVID-19, as described herein, comprises an effective amount of an apoptotic supernatant, as described herein in a pharmaceutically acceptable excipient.

In another embodiment, a composition disclosed herein and used in methods disclosed herein comprises apoptotic cells or an apoptotic cell supernatant, and a pharmaceutically acceptable excipient. In some embodiments, a composition comprising early cells or an apoptotic cell supernatant is used in methods disclosed herein for example for treating COVID-19 in a subject and or symptoms thereof.

In another embodiment, early apoptotic cells comprised in a composition comprise apoptotic cells in an early apoptotic state. In another embodiment, early apoptotic cells comprised in a composition are pooled third party donor cells. In another embodiment, an apoptotic cell supernatant comprised in a composition disclosed herein is collected from early apoptotic cells. In another embodiment, an apoptotic cell supernatant comprised in a composition disclosed herein, is collected pooled third-party donor cells.

In one embodiment, the additional pharmaceutical composition comprises a CTLA-4 blocking agent, which in one embodiment is Ipilimumab. In another embodiment, the additional pharmaceutical composition comprises an alpha-1 anti-trypsin, as disclosed herein, or a fragment thereof, or an analogue thereof. In another embodiment, the additional pharmaceutical composition comprises a tellurium-based compound, a disclosed herein. In another embodiment, the additional pharmaceutical composition comprises an immune modulating agent, as disclosed herein. In another embodiment, the additional pharmaceutical composition comprises a CTLA-4 blocking agent, an alpha-1 anti-trypsin or fragment thereof or analogue thereof, a tellurium-based compound, or an immune modulating compound, or any combination thereof.

In some embodiments, a composition comprises apoptotic cells and an additional agent. In some embodiments, a composition comprises apoptotic cells and an antibody or a functional fragment thereof. In some embodiments, a composition comprises apoptotic cells and a RtX antibody or a functional fragment thereof. In some embodiments, early apoptotic cells and an antibody or a functional fragment thereof may be comprised in separate compositions. In some embodiments, early apoptotic cells and an antibody or a functional fragment thereof may be comprised in the same composition.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of a compound to an organism.

In some embodiments, disclosed herein is a pharmaceutical composition for treating mild COVID-19 or symptoms thereof. In some embodiments, disclosed herein is a pharmaceutical composition for treating moderate COVID-19 or symptoms thereof. In some embodiments, disclosed herein is a pharmaceutical composition for treating severe COVID-19 or symptoms thereof. In some embodiments, disclosed herein is a pharmaceutical composition for treating critical COVID-19 or symptoms thereof. In some embodiments, disclosed herein is a pharmaceutical composition for increasing the survival of a subject suffering from mild COVID-19. In some embodiments, disclosed herein is a pharmaceutical composition for increasing the survival of a subject suffering from moderate COVID-19. In some embodiments, disclosed herein is a pharmaceutical composition for increasing the survival of a subject suffering from severe COVID-19. In some embodiments, disclosed herein is a pharmaceutical composition for increasing the survival of a subject suffering from critical COVID-19.

In some embodiments, a pharmaceutical composition comprises an early apoptotic cell population as described herein. In some embodiments, a pharmaceutical composition comprises an early apoptotic cell population as described herein, and a pharmaceutically acceptable excipient.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In some embodiments, the composition as disclosed herein comprises a therapeutic composition. In some embodiments, the composition as disclosed herein comprises a therapeutic efficacy.

### Formulations

Pharmaceutical compositions disclosed herein comprising early apoptotic cell populations, can be conveniently provided as sterile liquid preparations, e.g., isotonic aqueous solutions, suspensions, emulsions, dispersions, or viscous compositions, which may be buffered to a selected pH, Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the early apoptotic cell population described herein and utilized in practicing the methods disclosed herein, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the disclosure herein, however, any vehicle, diluent, or additive used would have to be compatible with the genetically modified immunoresponsive cells or their progenitors.

The compositions or formulations described herein can be isotonic, i.e., they can have the same osmotic pressure as blood and lacrimal fluid. The desired isotonicity of the compositions as disclosed herein may be accomplished using sodium chloride, or other pharmaceutically acceptable agents such as dextrose, boric acid, sodium tartrate, propylene glycol or other inorganic or organic solutes. Sodium chloride may be preferred particularly for buffers containing sodium ions.

Viscosity of the compositions, if desired, can be maintained at the selected level using a pharmaceutically acceptable thickening agent. Methylcellulose may be preferred because it is readily and economically available and is easy to work with.

Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions or formulations should be selected to be chemically inert and will not affect the viability or efficacy of the early apoptotic cell populations as described herein, for use in the methods disclosed herein. This will present no problem to those skilled in chemical and pharmaceutical principles, or problems can be readily avoided by reference to standard texts or by simple experiments (not involving undue experimentation), from this disclosure and the documents cited herein.

### Method of Use

In some embodiments, disclosed herein is a method of treating COVID-19 in a subject infected by SARS-CoV-2 virus, said method comprising administering a composition comprising an early apoptotic mononuclear-enriched cell population to the subject, wherein said administration treats COVID-19. In certain embodiments, methods of treating comprise treating, inhibiting, reducing the incidence of, ameliorating, or alleviating a symptom of COVID-19. In certain embodiments, methods of treating comprise preventing the appearance of symptoms of COVID-19. In some embodiments, methods of treating COVID-19 comprising administering a composition comprising an early apoptotic mononuclear-cell-enriched population results in a PCR negative result for SARS-CoV-2.

In some embodiments, methods of treating COVID-19 comprising administering a composition comprising an early apoptotic mononuclear-enriched cell population results in reduced stay in a hospital for the COVID-19 subject. In some embodiments, the stay is reduced by about 10%-90% compared with a subject not administered early apoptotic mononuclear-enriched cell population. In some embodiments, the stay is reduced by about 10%-90% compared with a subject not administered early apoptotic mononuclear-enriched cell population. In some embodiments, the stay is reduced by about 10%-50% compared with a subject not administered early apoptotic mononuclear-enriched cell population. In some embodiments, the stay is reduced by about 50%-90% compared with a subject not administered early apoptotic mononuclear-enriched cell population. In some embodiments, the stay is reduced by about 10%, 15%, 20%, 25%, 30%, 35%, 40%, 45%, 50%, 55%, 60%, 65%, 70%, 75%, 80%, 85%, or 90% compared with a subject not administered early apoptotic mononuclear-enriched cell population.

In some embodiments, methods of treating COVID-19 comprising administering a composition comprising an early apoptotic mononuclear-enriched cell population results in reduced stay in the intensive care unit (ICU) of a hospital for the COVID-19 subject. In some embodiments, the stay in ICU is reduced by about 10%-90% compared with a subject not administered early apoptotic mononuclear-enriched cell population. In some embodiments, the stay in ICU is reduced by about 10%-90% compared with a subject not administered early apoptotic mononuclear-enriched cell population. In some embodiments, the stay in ICU is reduced by about 10%-50% compared with a subject not administered early apoptotic mononuclear-enriched cell population. In some embodiments, the stay in ICU is reduced by about 50%-90% compared with a subject not administered early apoptotic mononuclear-enriched cell population. In some embodiments, the stay in ICU is reduced by about 10%, 15%, 20%, 25%, 30%, 35%, 40%, 45%, 50%, 55%, 60%, 65%, 70%, 75%, 80%, 85%, or 90% compared with a subject not administered early apoptotic mononuclear-enriched cell population.

In some embodiments, treating COVID-19 comprises treating a subject suffering from mild COVID-19. In some embodiments, treating COVID-19 comprises treating a subject suffering from moderate COVID-19. In some embodiments, treating COVID-19 comprises treating a subject suffering from severe COVID-19. In some embodiments, treating COVID-19 comprises treating a subject suffering from critical COVID-19.

With knowledge of the rapid progress of COVID-19, in some embodiments, treating COVID-19 comprises treating an asymptomatic subject infected with SARS-CoV-2 prophylactically so that the subject's symptoms are prevented, inhibited, or reduced in their progress towards a more severe form of COVID-19 (mild, moderate, severe, or critical). In some embodiments, treating COVID-19 comprises treating a subject suffering from mild COVID-19 prophylactically so that the subject's symptoms are prevented, inhibited, or reduced in their progress towards a more severe form of COVID-19 (moderate, severe, or critical). In some embodiments, treating COVID-19 comprises treating a subject suffering from moderate COVID-19 prophylactically so that the subject's symptoms are prevented, inhibited, or reduced in their progress towards a more severe form of COVID-19 (severe or critical). In some embodiments, treating COVID-19 comprises treating a subject suffering from severe COVID-19 so that the subject's symptoms are prevented, inhibited, or reduced in their progress towards a more severe form of COVID-19 (critical) or death.

In some embodiments, a method of treating comprises treating symptoms of COVID-19, wherein said symptoms comprises organ failure, organ dysfunction, organ damage, a cytokine storm, or a cytokine release syndrome, or a combination thereof. In some embodiments, methods of treating treat a single symptom. In some embodiments, methods of treating treat at least two symptoms. In some embodiments, methods of treating treat multiple symptoms.

A skilled artisan would appreciate that organ dysfunction may encompass a situation wherein an organ does not perform its expected function. Further, organ failure may encompass organ dysfunction to such a degree that normal homeostasis cannot be maintained without external clinical intervention.

In some embodiments, methods disclosed herein comprise treating COVID-19 in a subject experiencing organ dysfunction or failure, wherein the organ comprises a lung, a heart, a kidney, or a liver, or any combination thereof. In some embodiments, methods disclosed treat a symptom of organ dysfunction, damage, or failure, or a combination thereof. A combination of symptoms may occur when organ dysfunction or failure leads to organ damage. In some embodiments, organ damage is reparable. In some embodiments, organ damage is permanent. In some embodiments, treating organ dysfunction comprises reducing, slowing, inhibiting, reversing, or repairing said organ dysfunction, or any combination thereof. In some embodiments, treating organ damage comprises reducing, slowing, inhibiting, reversing, or repairing said organ damage, or any combination thereof.

In some embodiments, treating organ failure comprises reducing, slowing, inhibiting, reversing, or repairing said organ failure, or any combination thereof.

In some embodiments, methods disclosed treat a symptom of lung dysfunction, damage, or failure, or a combination thereof. In some embodiments, lung dysfunction comprises dyspnea, respiratory frequency greater than or equal to 30 breaths/min, measurements of SpO₂≤93%, PaO₂/FiO₂<300 mmHg, or lung infiltrates >50% within 24 to 48 hours, or any combination thereof. In some embodiments, lung dysfunction comprises dyspnea (shortness of breath). In some embodiments, lung dysfunction comprises respiratory frequency greater than or equal to 30 breaths/min. In some embodiments, lung dysfunction comprises measurements of SpO₂≤93%, PaO₂/FiO₂<300 mmHg. In some embodiments, lung dysfunction comprises lung infiltrates >50% within 24 to 48 hours. In some embodiments, lung dysfunction comprises acute respiratory distress syndrome (ARDS). In some embodiments, methods of treatments using early apoptotic cells treat respiratory complications.

In some embodiments, methods disclosed treat a symptom of heart dysfunction, damage, or failure, or a combination thereof. In some embodiments, methods disclosed treat a symptom of kidney dysfunction, damage, or failure, or a combination thereof. In some embodiments, methods disclosed treat a symptom of liver dysfunction, damage, or failure, or a combination thereof.

In some embodiments, methods disclosed treat a symptom of organ dysfunction, damage, or failure, or a combination thereof, comprising multiple organ dysfunction, damage, or failure. In some embodiments, multiple organ dysfunction, damage, or failure comprises dysfunction, damage, or failure of any combination of at least two of the lungs, heart, liver, or kidneys.

In some embodiments, treating COVID-19 in a subject infected by SARS-CoV-2 virus comprises treating, inhibiting, reducing the incidence of, ameliorating, or alleviating a symptom of COVID-19, wherein said symptom comprises organ failure. In some embodiments, treating COVID-19 in a subject infected by SARS-CoV-2 virus comprises treating, inhibiting, reducing the incidence of, ameliorating, or alleviating a symptom of COVID-19, wherein said symptom comprises organ dysfunction. In some embodiments, treating COVID-19 in a subject infected by SARS-CoV-2 virus comprises treating, inhibiting, reducing the incidence of, ameliorating, or alleviating a symptom of COVID-19, wherein said symptom comprises of organ damage. In some embodiments, treating COVID-19 in a subject infected by SARS-CoV-2 virus comprises treating, inhibiting, reducing the incidence of, ameliorating, or alleviating a symptom of COVID-19, wherein said symptom comprises acute multiple organ failure.

In some embodiments, treating COVID-19 in a subject infected by SARS-CoV-2 virus comprises administering early apoptotic cells to a subject suffering from organ dysfunction and results in treating, inhibiting, reducing the incidence of, ameliorating, or alleviating organ dysfunction. In some embodiments, treating COVID-19 in a subject infected by SARS-CoV-2 virus comprises administering early apoptotic cells to a subject suffering from organ failure and results in treating, inhibiting, reducing the incidence of, ameliorating, or alleviating organ failure. In some embodiments, treating COVID-19 in a subject infected by SARS-CoV-2 virus comprises administering early apoptotic cells to a subject suffering from organ damage and results in treating, inhibiting, reducing the incidence of, ameliorating, or alleviating organ damage. In some embodiments, treating COVID-19 in a subject infected by SARS-CoV-2 virus comprises administering early apoptotic cells to a subject suffering from acute multiple organ failure and results in treating, inhibiting, reducing the incidence of, ameliorating, or alleviating acute multiple organ failure.

In some embodiments, treating COVID-19 in a subject infected by SARS-CoV-2 virus comprises administering an early apoptotic supernatant to a subject suffering from organ dysfunction and results in treating, inhibiting, reducing the incidence of, ameliorating, or alleviating organ dysfunction. In some embodiments, treating COVID-19 in a subject infected by SARS-CoV-2 virus comprises administering an early apoptotic supernatant to a subject suffering from organ failure and results in treating, inhibiting, reducing the incidence of, ameliorating, or alleviating organ failure. In some embodiments, treating COVID-19 in a subject infected by SARS-CoV-2 virus comprises administering an early apoptotic supernatant to a subject suffering from organ damage and results in treating, inhibiting, reducing the incidence of, ameliorating, or alleviating organ damage. In some embodiments, treating COVID-19 in a subject infected by SARS-CoV-2 virus comprises administering an early apoptotic supernatant to a subject suffering from acute multiple organ failure and results in treating, inhibiting, reducing the incidence of, ameliorating, or alleviating acute multiple organ failure.

In some embodiments, organ failure during SARS-CoV-2 infection comprises failure of a vital organ, for example but not limited to lung, heart, kidney, liver, and blood organs. In some embodiments, multiple organ failure as a component of COVID-19 comprises failure of a combination of lung, the heart, a kidney, liver, and blood. In some embodiments, hematological aberrations during COVID-19 comprise thrombocytopenia, lymphopenia, neutropenia, or neutrophilia, or any combination thereof. In some embodiments, organ failure may be measured using standards known in the art including but not limited to the Sequential Organ Failure Assessment (SOFA) scores.

In some embodiments, treating COVID-19 in a subject in need comprises prevention, inhibiting, reducing the incidence of cardiovascular dysfunction. In some embodiments, treating COVID-19 in a subject in need comprises prevention, inhibiting, reducing the incidence of acute kidney injury. In some embodiments, treating COVID-19 in a subject in need comprises prevention, inhibiting, reducing the incidence of lung dysfunction. In some embodiments, treating COVID-19 in a subject in need comprises prevention, inhibiting, reducing the incidence of liver dysfunction. In some embodiments, treating COVID-19 in a subject in need comprises prevention, inhibiting, reducing the incidence of hematological aberrations. In some embodiments, treating COVID-19 in a subject in need comprises prevention, inhibiting, reducing the incidence of a combination of any of cardiovascular dysfunction, acute kidney injury, lung dysfunction, and hematological aberrations.

In some embodiments, administering early apoptotic mononuclear-enriched cells to a subject suffering from COVID-19 results in preventing, inhibiting, reducing the incidence of cardiovascular dysfunction. In some embodiments, administering early apoptotic mononuclear-enriched cells to a subject suffering from COVID-19 results in preventing, inhibiting, reducing the incidence of acute kidney injury. In some embodiments, administering early apoptotic mononuclear enriched cells to a subject suffering from COVID-19 results in preventing, inhibiting, reducing the incidence of lung dysfunction. In some embodiments, administering early apoptotic mononuclear-enriched cells to a subject suffering from COVID-19 results in preventing, inhibiting, reducing the incidence of liver dysfunction. In some embodiments, administering early apoptotic mononuclear-enriched cells to a subject suffering from COVID-19 results in preventing, inhibiting, reducing the incidence of hematological aberrations. In some embodiments, administering early apoptotic mononuclear enriched cells to a subject suffering from COVID-19 results in preventing, inhibiting, reducing the incidence of a combination of any of cardiovascular dysfunction, acute kidney injury, lung dysfunction, and hematological aberrations.

In some embodiments, administering an early apoptotic supernatant to a subject suffering from COVID-19 results in preventing, inhibiting, reducing the incidence of cardiovascular dysfunction. In some embodiments, administering an early apoptotic supernatant to a subject suffering from COVID-19 results in preventing, inhibiting, reducing the incidence of acute kidney injury. In some embodiments, administering an early apoptotic supernatant to a subject suffering from COVID-19 results in preventing, inhibiting, reducing the incidence of lung dysfunction. In some embodiments, administering an early apoptotic supernatant to a subject suffering from COVID-19 results in preventing, inhibiting, reducing the incidence of liver dysfunction. In some embodiments, administering an early apoptotic supernatant to a subject suffering from COVID-19 results in preventing, inhibiting, reducing the incidence of hematological aberrations. In some embodiments, administering an early apoptotic supernatant to a subject suffering from COVID-19 results in preventing, inhibiting, reducing the incidence of a combination of any of cardiovascular dysfunction, acute kidney injury, lung dysfunction, and hematological aberrations.

In some embodiments, administering early apoptotic mononuclear enriched cells to a subject suffering from COVID-19 results in treating, inhibiting, reducing the incidence of, ameliorating, or alleviating a symptom of COVID-19. In some embodiments, administering early apoptotic mononuclear enriched cells to a subject suffering from COVID-19 results in treating, inhibiting, reducing the incidence of, ameliorating, or alleviating a symptom of COVID-19, wherein said symptom comprises organ failure, organ dysfunction, organ damage, cytokine storm, a cytokine release syndrome, or a combination thereof. In some embodiments, administering early apoptotic mononuclear enriched cells to a subject suffering from COVID-19 results in treating, inhibiting, reducing the incidence of, ameliorating, or alleviating a symptom of COVID-19, wherein said symptom comprises organ failure, organ dysfunction, organ damage, cytokine storm, a cytokine release syndrome, or a combination thereof, compared with subjects not administered early apoptotic cells.

In some embodiments, administering an early apoptotic supernatant to a subject suffering from COVID-19 results in treating, inhibiting, reducing the incidence of, ameliorating, or alleviating a symptom of COVID-19. In some embodiments, administering an early apoptotic supernatant to a subject suffering from COVID-19 results in treating, inhibiting, reducing the incidence of, ameliorating, or alleviating a symptom of COVID-19, wherein said symptom comprises organ failure, organ dysfunction, organ damage, cytokine storm, a cytokine release syndrome, or a combination thereof. In some embodiments, administering an early apoptotic supernatant to a subject suffering from COVID-19 results in treating, inhibiting, reducing the incidence of, ameliorating, or alleviating a symptom of COVID-19, wherein said symptom comprises organ failure, organ dysfunction, organ damage, cytokine storm, a cytokine release syndrome, or a combination thereof, compared with subjects not administered an early apoptotic supernatant.

In some embodiments, administering early apoptotic mononuclear-enriched cells to a subject suffering from COVID-19 is highly effective in the treatment of COVID-19. In some embodiments, measure of an effective treatment of COVID-19 includes the percent of patients that recover from COVID-19 within a given timeframe. In some embodiments, measure of an effective treatment of COVID-19 includes the percent of patients that are released from intensive care compared with the percent of patients not administered early apoptotic cells. In some embodiments, a subject suffering from COVID-19 administered early apoptotic cells recovers more quickly than a subject suffering from COVID-19 and not administered early apoptotic cells. In some embodiments, a subject suffering from COVID-19 administered early apoptotic cells recovers more completely than a subject suffering from COVID-19 and not administered early apoptotic cells. In some embodiments, the mortality rate of patients suffering from COVID-19 and treated with early apoptotic cells is decreased, compared with patients not administered early apoptotic cells.

In some embodiments, a COVID-19 subject comprises a human. In some embodiments, a COVID-19 subject comprises a human adult. In some embodiments, a COVID-19 subject comprises a human child.

In some embodiments, methods of treating COVID-19 comprise treating mild COVID-19. In some embodiments, methods of treating COVID-19 comprise treating moderate COVID-19. In some embodiments, methods of treating COVID-19 comprise treating severe COVID-19. In some embodiments, methods of treating COVID-19 comprise treating critical COVID-19. The some embodiments, methods of treating COVID-19 comprise treating mild, moderate, severe, or critical COVID-19. The some embodiments, methods of treating COVID-19 comprise treating moderate, severe, or critical COVID-19. The some embodiments, methods of treating COVID-19 comprise treating severe or critical COVID-19.

In some embodiments, treating COVID-19 in a subject in need comprises treating, inhibiting, reducing the incidence of, ameliorating, or alleviating a cytokine storm. In some embodiments, treating COVID-19 in a subject in need comprises treating, inhibiting, reducing the incidence of, ameliorating, or alleviating a chemokine storm. comprises treating, inhibiting, reducing the incidence of, ameliorating, or alleviating a cytokine and chemokine storm.

In some embodiments, administering early apoptotic cells to a subject suffering from COVID-19 results in treating, inhibiting, reducing the incidence of, ameliorating, or alleviating a cytokine storm. In some embodiments, administering early apoptotic cells to a subject suffering from COVID-19 results in treating, inhibiting, reducing the incidence of, ameliorating, or alleviating a chemokine storm. In some embodiments, administering early apoptotic cells to a subject suffering from COVID-19 results in treating, inhibiting, reducing the incidence of, ameliorating, or alleviating a cytokine and chemokine storm.

In some embodiments, administering an early apoptotic supernatant to a subject suffering from COVID-19 results in treating, inhibiting, reducing the incidence of, ameliorating, or alleviating a cytokine storm. In some embodiments, administering an early apoptotic supernatant to a subject suffering from COVID-19 results in treating, inhibiting, reducing the incidence of, ameliorating, or alleviating a chemokine storm. In some embodiments, administering an early apoptotic supernatant to a subject suffering from COVID-19 results in treating, inhibiting, reducing the incidence of, ameliorating, or alleviating a cytokine and chemokine storm.

In some embodiments, treating COVID-19 in a subject in need comprises rebalancing the immune response in a subject. In some embodiments, treating COVID-19 in a subject in need comprises reducing secretion of pro-inflammatory cytokines. In some embodiments, treating COVID-19 in a subject in need comprises reducing secretion of pro-inflammatory cytokines/chemokines and anti-inflammatory cytokines/chemokines.

In some embodiments, administering early apoptotic cells to a subject suffering from COVID-19 results in rebalancing the immune response in a subject. In some embodiments, administering early apoptotic cells to a subject suffering from COVID-19 results in reducing secretion of pro-inflammatory cytokines. In some embodiments, administering early apoptotic cells to a subject suffering from COVID-19 results in reducing secretion of pro-inflammatory cytokines/chemokines and anti-inflammatory cytokines/chemokines.

In some embodiments, administering an early apoptotic supernatant to a subject suffering from COVID-19 results in rebalancing the immune response in a subject. In some embodiments, administering an early apoptotic supernatant to a subject suffering from COVID-19 results in reducing secretion of pro-inflammatory cytokines. In some embodiments, administering an early apoptotic supernatant to a subject suffering from COVID-19 results in reducing secretion of pro-inflammatory cytokines/chemokines and anti-inflammatory cytokines/chemokines.

In some embodiments, rebalancing the immune response comprises reducing the secretion of one or more proinflammatory cytokines, anti-inflammatory cytokines, chemokine, or immune modulator, or a combination thereof. In some embodiments, rebalancing the immune response comprises increasing the secretion of one or more anti-inflammatory cytokine or chemokine, or combination thereof. In some embodiments, rebalancing the immune response comprises reducing secretion of one or more pro- or anti-inflammatory cytokine or chemokine or immune modulator, and increasing one or more anti-inflammatory cytokine or chemokine.

In certain embodiments, methods of treating COVID-19 comprise increasing survival time of a COVID-19 subject, compared with a COVID-19 subject not administered early apoptotic mononuclear-cell-enriched population. In certain embodiments, methods of treating COVID-19 comprise increasing survival time of a COVID-19 subject, compared with a COVID-19 subject not administered an early apoptotic supernatant.

In certain embodiments, methods of treating symptoms of COVID-19 comprise increasing survival time of a COVID-19 subject, compared with a COVID-19 subject not administered early apoptotic mononuclear-cell-enriched population. In certain embodiments, methods of treating symptoms of COVID-19 comprise increasing survival time of a COVID-19 subject, compared with a COVID-19 subject not administered an early apoptotic supernatant.

In some embodiments, treating COVID-19 in a subject in need comprises a reduction in mortality of a subject suffering from COVID-19 and symptoms thereof. In some embodiments, treating COVID-19 in a subject in need comprises improving the survival time in the subject in need.

As skilled artisan would appreciate that treating COVID-19 may in certain embodiments, encompass treating symptoms of COVID-19.

In some embodiments, treating COVID-19 in a subject in need increase the survival time in said subject by greater than 60% compared with a subject not administered early apoptotic cells or an early apoptotic supernatant. In some embodiments, treating COVID-19 in a subject in need increase the survival time in said subject by greater than 70% compared with a subject not administered early apoptotic cells or an early apoptotic supernatant. In some embodiments, treating COVID-19 in a subject in need increase the survival time in said subject by greater than 80% compared with a subject not administered early apoptotic cells or an early apoptotic supernatant. In some embodiments, treating COVID-19 in a subject in need increase the survival time in said subject by greater than 90% compared with a subject not administered early apoptotic cells or an early apoptotic supernatant. In some embodiments, treating COVID-19 in a subject in need increase the survival time in said subject by greater than 95% compared with a subject not administered early apoptotic cells or an early apoptotic supernatant.

In some embodiments, treating COVID-19 in a subject in need increase the survival time in said subject by about 25-50%, compared with a subject not administered early apoptotic cells or a supernatant thereof. In some embodiments, treating COVID-19 in a subject in need increase the survival time in said subject by about 50%-100%, compared with a subject not administered early apoptotic cells or a supernatant thereof. In some embodiments, treating COVID-19 in a subject in need increase the survival time in said subject by about 80%-100%, compared with a subject not administered early apoptotic cells or a supernatant thereof. In some embodiments, methods of treating COVID-19 in a subject in need increase the survival time in said subject by about 80%, 90%, or 100% compared with a subject not administered early apoptotic cells or a supernatant thereof.

In some embodiments, method of treating COVID-19 in a subject in need increases the survival time in said subject by about 100%-2000%, compared with a subject not administered early apoptotic cells or a supernatant thereof. In some embodiments, method of treating COVID-19 in a subject in need increase the survival time in said subject by about 200%-300%, compared with a subject not administered early apoptotic cells or a supernatant thereof. In some embodiments, method of treating COVID-19 in a subject in need increase the survival time in said subject by greater than 100% compared with a subject not administered early apoptotic cells or a supernatant thereof. In some embodiments, method of treating COVID-19 in a subject in need increase the survival time in said subject by greater than 200% compared with a subject not administered early apoptotic cells or a supernatant thereof. In some embodiments, method of treating COVID-19 in a subject in need increase the survival time in said subject by greater than 300% compared with a subject not administered early apoptotic cells or a supernatant thereof. In some embodiments, method of treating COVID-19 in a subject in need increase the survival time in said subject by greater than 400% compared with a subject not administered early apoptotic cells or a supernatant thereof. In some embodiments, method of treating COVID-19 in a subject in need increase the survival time in said subject by greater than 500%, 600%, 700%, 800%, 900%, or 1000% compared with a subject not administered early apoptotic cells or a supernatant thereof.

In some embodiments, method of treating COVID-19 in a subject in need increase the survival time in said subject by about 100% compared with a subject not administered early apoptotic cells or a supernatant thereof. In some embodiments, method of treating COVID-19 in a subject in need increase the survival time in said subject by about 200%, 300%, 400%, 500%, 600%, 700%, 800%, 900%, or 1000%, compared with a subject not administered early apoptotic cells or a supernatant thereof.

In some embodiments, method of treating COVID-19 in a subject in need increase the survival time in said subject by about 100%-1000%, compared with a subject not administered early apoptotic cells or a supernatant thereof. In some embodiments, method of treating COVID-19 in a subject in need increase the survival time in said subject by about 100%-500%, compared with a subject not administered early apoptotic cells or a supernatant thereof. In some embodiments, method of treating COVID-19 in a subject in need increase the survival time in said subject by about 500%-1000%, compared with a subject not administered early apoptotic cells or a supernatant thereof. In some embodiments, method of treating COVID-19 in a subject in need increase the survival time in said subject by about 70-80%, compared with a subject not administered early apoptotic cells or a supernatant thereof. In some embodiments, method of treating COVID-19 in a subject in need increase the survival time in said subject by about 50% compared with a subject not administered early apoptotic cells or a supernatant thereof. In some embodiments, method of treating COVID-19 in a subject in need increase the survival time in said subject by about 60% compared with a subject not administered early apoptotic cells or a supernatant thereof. In some embodiments, method of treating COVID-19 in a subject in need increase the survival time in said subject by about 70% compared with a subject not administered early apoptotic cells or a supernatant thereof. In some embodiments, method of treating COVID-19 in a subject in need increase the survival time in said subject by about 80%, compared with a subject not administered early apoptotic cells or a supernatant thereof.

In some embodiments, a method described herein, decreasing or inhibiting cytokine production in a subject experiencing cytokine release syndrome or cytokine storm or vulnerable to a cytokine release syndrome or cytokine storm, decreases or inhibits cytokine production. In another embodiment, the method described herein decreases or inhibits pro-inflammatory cytokine production. In a further embodiment, the method described herein decreases or inhibits at least one pro-inflammatory cytokine.

In some embodiments, disclosed herein are methods of treating COVID-19 wherein the method inhibits or reduces the incidence of cytokine release syndrome or cytokine storm in a COVID-19 subject. In some embodiments, disclosed herein are methods of treating COVID-19 wherein the method inhibits or reduces the incidence cytokine production in a COVID-19 subject experiencing cytokine release syndrome or cytokine storm, said methods comprising the step of administering a composition comprising early apoptotic cells or a supernatant of early apoptotic cells. In another embodiment, disclosed herein are methods of treating cytokine release syndrome or cytokine storm in a COVID-19 subject. In another embodiment, disclosed herein are methods of preventing cytokine release syndrome or cytokine storm in a COVID-19 subject. In another embodiment, disclosed herein are methods of alleviating cytokine release syndrome or cytokine storm in a COVID-19 subject. In another embodiment, disclosed herein are methods of ameliorating cytokine release syndrome or cytokine storm in a COVID-19 subject.

A skilled artisan would appreciate that the term “production” as used herein in reference to a cytokine, may encompass expression of the cytokine as well as secretion of the cytokine from a cell. In one embodiment, increased production of a cytokine results in increased secretion of the cytokine from the cell. In an alternate embodiment, decreased production of a cytokine results in decreased secretion of the cytokine from the cell. In another embodiment, methods disclosed herein decrease secretion of at least one cytokine. In another embodiment, methods disclosed herein decrease secretion of IL-6. In another embodiment, methods disclosed herein increase secretion of at least one cytokine. In another embodiment, methods disclosed herein increase secretion of IL-2.

In another embodiment, a cell secreting at least one cytokine is a tumor cell. In another embodiment, a cell secreting at least one cytokine is a T-cell. In another embodiment, a cell secreting at least one cytokine is an immune cell. In another embodiment, a cell secreting at least one cytokine is a macrophage. In another embodiment, a cell secreting at least one cytokine is a B cell lymphocyte. In another embodiment, a cell secreting at least one cytokine is a mast cell. In another embodiment, a cell secreting at least one cytokine is an endothelial cell. In another embodiment, a cell secreting at least one cytokine is a fibroblast. In another embodiment, a cell secreting at least one cytokine is a stromal cell. A skilled artisan would recognize that the level of cytokines may be increased or decreased in cytokine secreting cells depending on the environment surrounding the cell.

In yet another embodiment, an additional agent used in the methods disclosed herein increases secretion of at least one cytokine. In yet another embodiment, an additional agent used in the methods disclosed herein maintains secretion of at least one cytokine. In still another embodiment, an additional agent used in the methods disclosed herein does not decrease secretion of at least one cytokine. In another embodiment, an additional agent used in the methods disclosed herein increases secretion of IL-2. In another embodiment, an additional agent used in the methods disclosed herein increases secretion of IL-2R. In another embodiment, an additional agent used in the methods disclosed herein maintains secretion levels of IL-2. In another embodiment, an additional agent used in the methods disclosed herein maintains secretion levels of IL-2R. In another embodiment, an additional agent used in the methods disclosed herein does not decrease secretion levels of IL-2R. In another embodiment, an additional agent used in the methods disclosed herein maintains or increases secretion levels of IL-2. In another embodiment, an additional agent used in the methods disclosed herein maintains or increases secretion levels of IL-2R. In another embodiment, an additional agent used in the methods disclosed herein does not decrease secretion levels of IL-2R.

In still a further embodiment, an additional agent used in the methods disclosed herein decreases secretion of IL-6. In another embodiment, an additional agent used in the methods disclosed herein maintains, increases, or does not decrease secretion levels of IL-2 while decreasing secretion of IL-6. In another embodiment, an additional agent used in the methods disclosed herein maintains, increases, or does not decrease secretion levels of IL-2R while decreasing secretion of IL-6.

### Administration

In one embodiment, methods disclosed herein administer compositions comprising early apoptotic mononuclear-enriched cells as disclosed herein. In another embodiment, methods disclosed herein administer compositions comprising early apoptotic cell supernatants as disclosed herein.

In some embodiments, a method disclosed herein comprises administering an early apoptotic cell population comprising a mononuclear enriched cell population, as described in detail above. In some embodiments, a method disclosed herein comprises administering an early apoptotic cell population comprising a stable population cell, wherein said cell population is stable for greater than 24 hours (See for example, Example 1). In some embodiments, a method disclosed herein comprises administering an early apoptotic cell population comprising a population of cells devoid of cell aggregates. Early apoptotic cell populations devoid of aggregates and methods of making them have been described in detail herein.

In some embodiments, a method disclosed herein comprises administering an autologous early apoptotic cell population to a subject in need. In some embodiments, a method disclosed herein comprises administering an allogeneic early apoptotic cell population to a subject in need. In some embodiments, administering comprises a single infusion of the early apoptotic mononuclear-cell-enriched population. In some embodiments, administering comprises a single infusion of an early apoptotic supernatant. In some embodiments, administering comprises multiple infusions of the early apoptotic mononuclear-cell-enriched population. In some embodiments, administering comprises multiple infusions of an early apoptotic supernatant.

In some embodiments, methods of administration of early apoptotic cell populations or supernatants thereof, or compositions thereof comprise administering a single infusion of said apoptotic cell population or composition thereof. In some embodiments, a single infusion may be administered as a prophylactic to a subject predetermined to be at risk for COVID-19. In some embodiments, a single infusion may be administered as a prophylactic to an asymptomatic COVID-19 subject. In some embodiments, a single infusion may be administered to a COVID-19 subject experiencing mild, moderate, severe, or critical COVID-19. In some embodiments, a single infusion may be administered as a prophylactic to an asymptomatic COVID-19 subject in order to prevent, reduce the risk of, or delay the appearance of mild, moderate, severe, or critical symptoms of COVID-19.

In some embodiments, methods of administration of early apoptotic cell populations or supernatants thereof, or compositions thereof comprise administering multiple infusions of said apoptotic cell population or supernatants thereof, or composition thereof. In some embodiments, multiple infusions may be administered as a prophylactic to a subject predetermined to be at risk for COVID-19. In some embodiments, multiple infusions may be administered as a prophylactic to an asymptomatic COVID-19 subject. In some embodiments, multiple infusions may be administered as a prophylactic to a COVID-19 subject in order to prevent, reduce the risk of, or delay the appearance of moderate, severe, or critical symptoms.

In some embodiments, multiple infusions comprise at least two infusions. In some embodiments, multiple infusions comprise 2 infusions. In some embodiments, multiple infusions comprise more than 2 infusions. In some embodiments, multiple infusions comprise at least 3 infusions. In some embodiments, multiple infusions comprise 3 infusions. In some embodiments, multiple infusions comprise more than 3 infusions. In some embodiments, multiple infusions comprise at least 4 infusions. In some embodiments, multiple infusions comprise 4 infusions. In some embodiments, multiple infusions comprise more than 4 infusions. In some embodiments, multiple infusions comprise at least 5 infusions. In some embodiments, multiple infusions comprise 5 infusions. In some embodiments, multiple infusions comprise more than 5 infusions. In some embodiments, multiple infusions comprise at least six infusions. In some embodiments, multiple infusions comprise 6 infusions. In some embodiments, multiple infusions comprise more than 6 infusions. In some embodiments, multiple infusions comprise at least 7 infusions. In some embodiments, multiple infusions comprise 7 infusions. In some embodiments, multiple infusions comprise more than 7 infusions. In some embodiments, multiple infusions comprise at least 8 infusions. In some embodiments, multiple infusions comprise 8 infusions. In some embodiments, multiple infusions comprise more than 8 infusions. In some embodiments, multiple infusions comprise at least nine infusions. In some embodiments, multiple infusions comprise 9 infusions. In some embodiments, multiple infusions comprise more than 9 infusions. In some embodiments, multiple infusions comprise at least 10 infusions. In some embodiments, multiple infusions comprise 10 infusions. In some embodiments, multiple infusions comprise more than 10 infusions.

In some embodiments, multiple infusions comprise smaller amounts of early apoptotic cell, wherein the total dosage of cells administered is the sum of the infusions.

In some embodiments, multiple infusions are administered over a period of hours. In some embodiments, multiple infusions are administered over a period of days. In some embodiments, multiple infusions are administered over a period of hours, wherein there is at least 12 hours between infusions. In some embodiments, multiple infusions are administered over a period of hours, wherein there is at least 24 hours between infusions. In some embodiments, multiple infusions are administered over a period of hours, wherein there is at least a day between infusions. In some embodiments, multiple infusions are administered over a period of hours, wherein there is at least two days between infusions. In some embodiments, multiple infusions are administered over a period of hours, wherein there is at least three days between infusions. In some embodiments, multiple infusions are administered over a period of hours, wherein there is at least four days between infusions. In some embodiments, multiple infusions are administered over a period of hours, wherein there is at least five days between infusions. In some embodiments, multiple infusions are administered over a period of hours, wherein there is at least six days between infusions. In some embodiments, multiple infusions are administered over a period of hours, wherein there is at least seven days between infusions. In some embodiments, multiple infusions are administered over a period of hours, wherein there is at least a week between infusions. In some embodiments, multiple infusions are administered over a period of hours, wherein there is at least two weeks between infusions.

In some embodiments, the number of cells in multiple infusions is essentially equivalent one to the other. In some embodiments, the number of cells in multiple infusions is different one to the other.

In some embodiments, the methods described herein further comprise administering an additional agent for the treatment of COVID-19 and symptoms thereof, to said subject. In some embodiments, the methods comprise a step of administering an additional therapy.

In some embodiments, an additional agent or therapy is administered concurrent or essentially concurrent with the early apoptotic cells or supernatant. In some embodiments, an additional agent or therapy is administered prior to administration of the early apoptotic cells or supernatant. In some embodiments, an additional agent or therapy is administered following the administration of the early apoptotic cells or supernatant. In some embodiments, an additional agent is comprised in the same composition as the early apoptotic cells or supernatant. In some embodiments, an additional agent is comprised in a different composition from the early apoptotic cells or supernatant thereof.

In some embodiments, methods disclosed herein comprise a first-line therapy.

A skilled artisan would appreciate that the term “first-line therapy” may encompass the first treatment given for a disease. When used by itself, first-line therapy is the one accepted as the best treatment. If it doesn't cure the disease or it causes severe side effects, other treatment may be added or used instead. Also called induction therapy, primary therapy, and primary treatment.

In some embodiments, methods disclosed herein comprise an adjuvant therapy.

A skilled artisan would appreciate that the term “adjuvant therapy” may encompass a treatment that is given in addition to the primary or initial treatment. In some embodiments, adjuvant therapy may comprise a treatment given prior to the primary treatment in preparation of a further treatment. In some embodiments, adjuvant therapy may comprise an additional treatment given after the primary treatment to lower the risk of moderate or severe or critical COVID-19 symptoms. In some embodiments, adjuvant therapy may comprise an additional treatment given after the primary treatment to lower the risk of severe or critical COVID-19 symptoms.

In some embodiments, administration of early apoptotic cells or a supernatant thereof to a subject experiencing COVID-19 comprises intravenous administration. In some embodiments, administration of apoptotic cells to a subject experiencing COVID-19 comprising intravenous administration following an initial standard of care treatment.

In some embodiments, administration of an early apoptotic cells or a supernatant thereof to a subject infected with the SARS-CoV-2 virus comprises administration between 12-24 hours post diagnosis of COVID-19. In some embodiments, administration of an early apoptotic cells or a supernatant thereof to a subject infected with the SARS-CoV-2 virus comprises administration between 12-36 hours post diagnosis of COVID-19. In some embodiments, administration of In some embodiments, administration of an early apoptotic cells or a supernatant thereof to a subject infected with the SARS-CoV-2 virus comprises administration between 24-36 hours post diagnosis of COVID-19. In some embodiments, administration of an early apoptotic cells or a supernatant thereof to a subject infected with the SARS-CoV-2 virus comprises administration between 12-18 hours post diagnosis of COVID-19. In some embodiments, administration of an early apoptotic cells or a supernatant thereof to a subject infected with the SARS-CoV-2 virus comprises administration between 18-24 hours post diagnosis of COVID-19. In some embodiments, administration of an early apoptotic cells or a supernatant thereof to a subject infected with the SARS-CoV-2 virus comprises administration between 18-30 hours post diagnosis of COVID-19. In some embodiments, administration of an early apoptotic cells or a supernatant thereof to a subject infected with the SARS-CoV-2 virus comprises administration between 24-30 hours post diagnosis of COVID-19. In some embodiments, administration of an early apoptotic cells or a supernatant thereof to a subject infected with the SARS-CoV-2 virus comprises administration between 24-36 hours post diagnosis of COVID-19.

In some embodiments, administration of early apoptotic cells or a supernatant thereof to a subject experiencing COVID-19 comprises administration about 12 hours post diagnosis of COVID-19. In some embodiments, administration of early apoptotic cells or a supernatant thereof to a subject experiencing COVID-19 comprises administration about 13, 14, 15, 16, 17, 18, 19, 20, 21, 22, 23, 24, 25, 26, 27, 28, 29, 30, 31, 32, 33, 34, 35, or hours post diagnosis of COVID-19. In some embodiments, administration of early apoptotic cells or a supernatant thereof to a subject experiencing COVID-19 comprises administration within 24 hours±6 hours post diagnosis of COVID-19.

In some embodiments, the response of a subject suffering COVID-19 and administered a composition comprising early apoptotic cells or a supernatant thereof comprises a dose response.

In some embodiments, a dose of about 140×10⁶-210×10⁶ early apoptotic cells are administered. In some embodiments, a dose of about 10-100×10⁶ early apoptotic cells is administered. In some embodiments, a dose of about 20×10⁶ early apoptotic cells is administered. In some embodiments, a dose of about 30×10⁶ early apoptotic cells is administered. In some embodiments, a dose of about 40×10⁶ early apoptotic cells is administered. In some embodiments, a dose of about 50×10⁶ early apoptotic cells is administered. In some embodiments, 60×10⁶ early apoptotic cells is administered. In some embodiments, a dose of about 60×10⁶ early apoptotic cells is administered. In some embodiments, a dose of about 70×10⁶ early apoptotic cells is administered. In some embodiments, a dose of about 80×10⁶ early apoptotic cells is administered. In some embodiments, a dose of about 90×10⁶ early apoptotic cells is administered. In some embodiments, a dose of about 1-15×10⁷ early apoptotic cells is administered. In some embodiments, a dose of about 10×10⁷ early apoptotic cells is administered. In some embodiments, a dose of about 15×10⁷ early apoptotic cells is administered.

In some embodiments, a dose of 10×10⁶ early apoptotic cells is administered. In another embodiment, a dose of 10×10⁷ early apoptotic cells is administered. In another embodiment, a dose of 10×10⁸ early apoptotic cells is administered. In another embodiment, a dose of 10×10⁹ early apoptotic cells is administered. In another embodiment, a dose of 10×10¹⁰ early apoptotic cells is administered. In another embodiment, a dose of 10×10¹¹ early apoptotic cells is administered. In another embodiment, a dose of 10×10¹² early apoptotic cells is administered. In another embodiment, a dose of 10×10⁵ early apoptotic cells is administered. In another embodiment, a dose of 10×10⁴ early apoptotic cells is administered. In another embodiment, a dose of 10×10³ early apoptotic cells is administered. In another embodiment, a dose of 10×10² early apoptotic cells is administered.

In some embodiments, a high dose of early apoptotic cells is administered. In some embodiments, a dose of 35×10⁶ early apoptotic cells is administered. In another embodiment, a dose of 210×10⁶ early apoptotic cells is administered. In another embodiment, a dose of 70×10⁶ early apoptotic cells is administered. In another embodiment, a dose of 140×10⁶ early apoptotic cells is administered. In another embodiment, a dose of 35-210×10⁶ early apoptotic cells is administered.

In some embodiments, a single dose of early apoptotic cells is administered. In some embodiments, multiple doses of early apoptotic cells are administered. In some embodiments, 2 doses of early apoptotic cells are administered. In some embodiments, 3 doses of early apoptotic cells are administered. In some embodiments, 4 doses of early apoptotic cells are administered. In some embodiments, 5 doses of early apoptotic cells are administered. In some embodiments, 6 doses of early apoptotic cells are administered. In some embodiments, 7 doses of early apoptotic cells are administered. In some embodiments, 8 doses of early apoptotic cells are administered. In some embodiments, 9 doses of early apoptotic cells are administered. In some embodiments, more than 9 doses of early apoptotic cells are administered. In some embodiments, multiple doses of early apoptotic cells are administered.

In some embodiments, the early apoptotic cells may be administered by any method known in the art including, but not limited to, intravenous, subcutaneous, intranodal, intrathecal, intrapleural, intraperitoneal and directly to the thymus.

In another embodiment, a dose of early apoptotic cell supernatant derived from the co-culture of about 10×10⁶ early apoptotic cells is administered. In another embodiment, a dose derived from 10×10⁷ early apoptotic cells is administered. In another embodiment, a dose derived from 10×10⁸ early apoptotic cells is administered. In another embodiment, a dose derived from 10×10⁹ early apoptotic cells is administered. In another embodiment, a dose derived from 10×10¹⁰ early apoptotic cells is administered. In another embodiment, a dose derived from 10×10¹¹ early apoptotic cells is administered. In another embodiment, a dose derived from 10×10¹² early apoptotic cells is administered. In another embodiment, a dose derived from 10×10⁵ early apoptotic cells is administered. In another embodiment, a dose derived from 10×10⁴ early apoptotic cells is administered. In another embodiment, a dose derived from 10×10³ early apoptotic cells is administered. In another embodiment, a dose derived from 10×10² early apoptotic cells is administered.

In some embodiments, a dose of early apoptotic cell supernatant derived from 35×10⁶ early apoptotic cells is administered. In another embodiment, a dose derived from 210×10⁶ early apoptotic cells is administered. In another embodiment, a dose derived from 70×10⁶ early apoptotic cells is administered. In another embodiment, a dose derived from 140×10⁶ early apoptotic cells is administered. In another embodiment, a dose derived from 35-210×10⁶ early apoptotic cells is administered.

In some embodiments, the early apoptotic cell supernatant, or composition comprising said early apoptotic cell supernatant, may be administered by any method known in the art including, but not limited to, intravenous, subcutaneous, intranodal, intrathecal, intrapleural, intraperitoneal and directly to the thymus, as discussed in detail herein.

In another embodiment, early apoptotic cells or early apoptotic cell supernatant may be administered therapeutically, once cytokine release syndrome has occurred. In one embodiment, early apoptotic cells or supernatant may be administered once cytokine release leading up to or attesting to the beginning of cytokine release syndrome is detected. In one embodiment, early apoptotic cells or supernatant can terminate the increased cytokine levels, or the cytokine release syndrome, and avoid its sequelae.

In another embodiment, early apoptotic cells or apoptotic cell supernatant may be administered therapeutically, at multiple time points. In another embodiment, administration of early apoptotic cells or apoptotic cell supernatant is at least at two time points described herein. In another embodiment, administration of early apoptotic cells or early apoptotic cell supernatant is at least at three time points described herein. In another embodiment, administration of early apoptotic cells or apoptotic cell supernatant is prior to CRS or a cytokine storm, and once cytokine release syndrome has occurred, and any combination thereof.

In one embodiment, early apoptotic cells are heterologous to the subject. In one embodiment, early apoptotic cells are derived from one or more donors. In one embodiment, early apoptotic cells are derived from one or more bone marrow donors. In another embodiment, early apoptotic cells are derived from one or more blood bank donations. In one embodiment, the donors are matched donors. In another embodiment, early apoptotic cells are from unmatched third-party donors. In one embodiment, early apoptotic cells are universal allogeneic apoptotic cells. In another embodiment, early apoptotic cells are from a syngeneic donor. In another embodiment, early apoptotic cells are from pooled third-party donor cells. In one embodiment, the donor is a bone marrow donor. In another embodiment, the donor is a blood bank donor. In another embodiment, early apoptotic cells are autologous to the subject. In this embodiment, the patient's own cells are used.

According to some embodiments, the therapeutic mononuclear-enriched cell preparation disclosed herein, or the early apoptotic cell supernatant is administered to the subject systemically. In another embodiment, administration is via the intravenous route. Alternately, the therapeutic mononuclear enriched cell or supernatant may be administered to the subject according to various other routes, including, but not limited to, the parenteral, intraperitoneal, intra-articular, intramuscular and subcutaneous routes.

According to some embodiments, the therapeutic mononuclear-enriched cell preparation disclosed herein, or the additional agent is administered to the subject systemically. In another embodiment, administration is via the intravenous route. Alternately, the therapeutic mononuclear enriched cell or the additional agent may be administered to the subject according to various other routes, including, but not limited to, the parenteral, intraperitoneal, intra-articular, intramuscular and subcutaneous routes.

In one embodiment, the preparation is administered in a local rather than systemic manner, for example, via injection of the preparation directly into a specific region of a patient's body.

In another embodiment, the therapeutic mononuclear enriched cells or supernatant are administered to the subject suspended in a suitable physiological buffer, such as, but not limited to, saline solution, PBS, HBSS, and the like. In addition, the suspension medium may further comprise supplements conducive to maintaining the viability of the cells. In another embodiment, the additional agent is administered to the subject suspended in a suitable physiological buffer, such as, but not limited to, saline solution, PBS, HBSS, and the like.

According to some embodiments the pharmaceutical composition is administered intravenously. According to another embodiment, the pharmaceutical composition is administered in a single dose. According to alternative embodiments the pharmaceutical composition is administered in multiple doses. According to another embodiment, the pharmaceutical composition is administered in two doses. According to another embodiment, the pharmaceutical composition is administered in three doses. According to another embodiment, the pharmaceutical composition is administered in four doses. According to another embodiment, the pharmaceutical composition is administered in five or more doses. According to some embodiments, the pharmaceutical composition is formulated for intravenous injection.

In some embodiments, a composition as disclosed herein is administered once. In another embodiment, the composition is administered twice. In another embodiment, the composition is administered three times. In another embodiment, the composition is administered four times. In another embodiment, the composition is administered at least four times. In another embodiment, the composition is administered more than four times.

A skilled artisan would recognize that an “effective amount” (or, “therapeutically effective amount”) may encompass an amount sufficient to effect a beneficial or desired clinical result upon treatment, for example but not limited to treating a symptom of COVID-19. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease or symptoms thereof, for example but not limited to respiratory distress. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

The skilled artisan can readily determine the number of cells and optional additives, vehicles, and/or carrier in compositions and to be administered in methods disclosed herein. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %. In another embodiment about 0.0001 to about 1 wt %. In still another embodiment, about 0.0001 to about 0.05 wt % or about 0.001 to about 20 wt %. In a further embodiment, about 0.01 to about 10 wt %. In another embodiment, about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. The time for sequential administrations can be ascertained without undue experimentation.

In some embodiments, the term “comprise” may encompass the inclusion of other components of the composition which affect the efficacy of the composition that may be known in the art. In some embodiments, the term “consisting essentially of” comprises a composition, which has early apoptotic cells or an early apoptotic cell supernatant. However, other components may be included that are not involved directly in the utility of the composition. In some embodiments, the term “consisting” encompasses a composition having early apoptotic cells or an early apoptotic cell supernatant as disclosed herein, in any form or embodiment as described herein.

A skilled artisan would appreciate that the term “about”, may encompass a deviance of between 0.0001-5% from the indicated number or range of numbers. Further, it may encompass a deviance of between 1-10% from the indicated number or range of numbers. In addition, it may encompass a deviance of up to 25% from the indicated number or range of numbers.

A skilled artisan would appreciate that the singular form “a”, “an” and “the” include plural references unless the context clearly dictates otherwise. For example, the term “an agent” or “at least an agent” may include a plurality of agents, including mixtures thereof.

In some embodiment, “treating” comprises therapeutic treatment and “preventing” comprises prophylactic or preventative measures, wherein the object is to prevent or lessen the targeted pathologic condition or disorder as described hereinabove. Thus, in some embodiments, treating may include directly affecting or curing, suppressing, inhibiting, preventing, reducing the severity of, delaying the onset of, reducing symptoms associated with COVID-19. Thus, in some embodiments, “treating,” “ameliorating,” and “alleviating” refer inter alia to delaying progression, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A skilled artisan would appreciate that the term “treatment” may encompass clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder. In some embodiments, improved prognosis comprises reduced hospital stay.

A skilled artisan would appreciate that the term “subject” may encompass a vertebrate, in some embodiments, to a mammal, and in some embodiments, to a human.

A skilled artisan would appreciate that the term “effective amount” may encompass an amount sufficient to have a therapeutic effect. In some embodiments, an “effective amount” is an amount sufficient to arrest, ameliorate, or inhibit the continued proliferation, growth, or metastasis (e.g., invasion, or migration) of a neoplasia.

Throughout this application, various embodiments disclosed herein may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

The following examples are presented in order to more fully illustrate embodiments disclosed herein. They should in no way be construed, however, as limiting the broad scope of the disclosure.

### EXAMPLES

### Example 1: Apoptotic Cell Production

Objective: To produce early apoptotic cells.

Methods: Methods of making populations of early-apoptotic cells have been well documented in International Publication No. WO 2014/087408 and United States Application Publication No. US2015/0275175-A1, see for example, the Methods section preceding the Examples at “Early apoptotic cell population Preparation” and “Generation of apoptotic cells” (paragraphs [0223] through [0288]), and Examples 11, 12, 13, and 14, which are incorporated herein in their entirety).

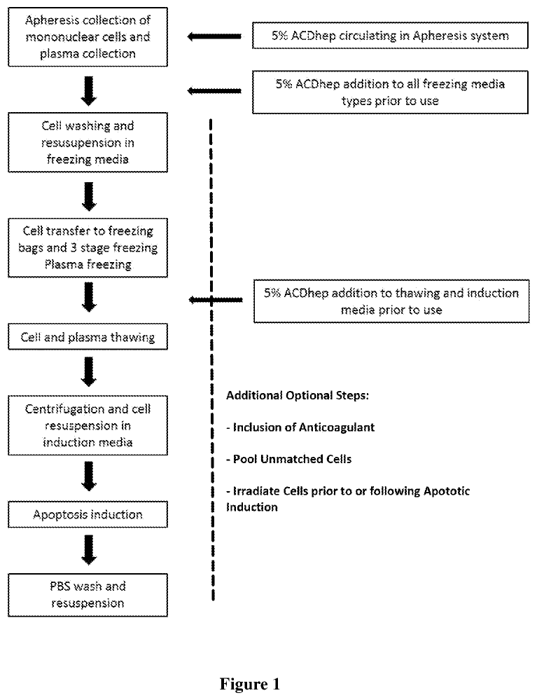

The flow chart presented in FIG. 1 provides an overview of one embodiment of the steps used during the process of producing a population of early apoptotic cells, wherein anticoagulants were included in the thawing and induction of apoptosis steps. As is described in detailed in Example 14 of International Publication No. WO 2014/087408 and United States Application Publication No. US US-2015-0275175-A1, early apoptotic cell populations were prepared wherein anti-coagulants were added at the time of freezing, or at the time of incubation, or at the time of freezing and at the time of incubation. The anticoagulant used was acid-citrate dextrose, NIH Formula A (ACD formula A) was supplemented with 10 U/ml heparin to a final concentration of 5% ACD of the total volume and 0.5 U/ml heparin.

Briefly: The cells were collected and then frozen with addition of 5% anticoagulant citrate dextrose formula A and 10 U/ml heparin (ACDhep) to the freezing media. Thawing, incubation in an apoptosis induction media containing 5% ACDhep, and final product preparation were performed in a closed system.

Apoptosis and viability analysis, potency assay, and cell population characterization were performed in each experiment. In order to establish consistence in production of the early apoptotic cell product, the final product (FP) of initial batches of apoptotic cells were stored at 2-8° C. and examined at t0, t24 h, t48 h and t72 h. At each point apoptosis analysis, short potency assay (Applicants CD14+ frozen cells), trypan blue measurement and cell population characterization were performed. The FP was tested for cell count to assess average cell loss during storage and apoptosis and viability analysis.

The methods sections cited above and Example 11 of International Publication No. WO 2014/087408 and United States Application Publication No. US US-2015-0275175-A1 provide details of preparing other embodiment of apoptotic cell populations in the absence of anti-coagulants, and are incorporated herein in full.

Methods of preparing irradiated apoptotic cells: Similar methods were used to prepare an inactivated apoptotic cell population, wherein a mononuclear early apoptotic cell population comprises a decreased percent of non-quiescent non-apoptotic cells, or a population of cells having a suppressed cellular activation of any living non-apoptotic cells, or a population of cells having a reduced proliferation of any living non-apoptotic cells, or any combination thereof.

Briefly, an enriched mononuclear cell fraction was collected via leukapheresis procedure from healthy, eligible donors. Following apheresis completion, cells were washed and resuspended with freezing media comprising 5% Anticoagulant Citrate Dextrose Solution-Formula A (ACD-A) and 0.5 U\ml heparin. Cell were then gradually frozen and transferred to liquid nitrogen for long term storage.

For preparation of irradiated early mononuclear enriched apoptotic cells derived from PBMC, cryopreserved cells were thawed, washed and resuspended with apoptosis induction media comprising 5% ACD-A, 0.5 U\ml heparin sodium and 50 μg/ml methylprednisolone. Cells were then incubated for 6 hours at 37° C. in 5% CO₂. At the end of incubation, cells were collected, washed and resuspended in Hartmann's solution using a cell processing system (Fresenius Kabi, Germany). Following manufacturing completion, ApoCell were irradiated at 4000 cGy using g-camera at the radiotherapy unit, Hadassah Ein Kerem. Apoptosis and viability of ApoCell determined using AnnexinV and PI (MBL, MA, USA) staining (≥40% and ≤15%, respectively) via Flow cytometer. Results analyzed using FCS express software. Thus, the early apoptotic cells were irradiated after they were prepared (after induction of apoptosis).

This irradiated Apocell population is considered to include early apoptotic cells, wherein any viable cells present have suppressed cellular activity and reduced or no proliferation capabilities. In certain cases, the Apocell population has no viable non-apoptotic cells.

Results: The stability of the FP produced with inclusion of anticoagulant at freezing and incubation (apoptotic induction) and then stored at 2-8° C. are shown below in Table 3.

TABLE 3
Cell count*-performed using a MICROS 60 hematology analyzer.

| FP Time point | Cell concentration (×l0⁶ cells\ml) | % of cell loss |
|---|---|---|
| t0 | 20.8 | NA |
| t24 h | 20.0 | −3.85 |
| t48 h | 20.0 | −3.85 |
| t72 h | 19.7 | −5.3 |

*Results Representative of 6 (six) experiments.

When manufacturing the cells without including an anticoagulant in the induction medium, cells were stable for 24 hours and less stable thereafter. Use of anticoagulants unexpectedly extended the stability of the apoptotic cell population for at least 72 hours, as shown in Table 3.

TABLE 4
Trypan blue measurement

| FP Time point | trypan blue positive cells (%) |
|---|---|
| t0 | 3.0 |
| t24 h | 5.9 |
| t48 h | 5.2 |
| t72 h | 6.5 |

The results of Table 4 show viability of the FP remained high for at least 72 hours.

TABLE 5
Apoptosis analysis-(AnPI staining) performed using
Flow Cytometry

| FP Time | 1.5 mM Ca | | |
|---|---|---|---|
| point | An-PI− (%) | An+PI− (%) | An+PI+ (%) |
| t0 | 44.3 | 50.9 | 4.8 |
| t24 h | 39.0 | 55.9 | 5.1 |
| t48 h | 34.8 | 60.1 | 5.1 |
| t72 h | 33.4 | 60.5 | 6.1 |

The data in Table 5 confirms that the majority of cells in the population produced are in early apoptosis, wherein the percent of cells in the population in early apoptosis (An+PI−) was greater than 50% and in some instances greater than 60%. The cell population produced comprises a minimal percent of cells in late apoptosis or dead cells (less than or equal to 6%). See also Table 5 below.

The results of Table 5 show that the percent apoptotic cells versus necrotic cells was maintained over at extended time period of at least 72 hours post preparation of the cells, as was the percentage of early apoptotic cells.

Inclusion of anticoagulants both at the time of freezing and during induction of apoptosis resulted in the most consistently high yield of stable early-apoptotic cells (average yield of early apoptotic cells 61.3±2.6% % versus 48.4±5.0%, wherein 100% yield is based on the number of cells at freezing). This high yield was maintained even after 24 hours storage at 2-8° C.

Next a comparison was made between the inclusion of the anticoagulant at freezing or thawing or both, wherein percent (%) recovery was measured as well as stability. Anticoagulant was included in the apoptotic incubation mix for all populations. Table 6 presents the results of these studies.

TABLE 6
Yield and stability comparison of final products (FP) manufactured from cells
collected, with (“+”) or without (“−”) addition of anticoagulant
during freezing (“F”) and thawing (“Tha”)

| | # of | | | | | | | | |
|---|---|---|---|---|---|---|---|---|---|
| | (×10⁹, | % Cell Recovery in Final Product of Collected Cells | | | | | | | |
| | 100%) | FP t0 | | | | FP t24 h* | | | |
| Donor | Collected | F−/ | F−/ | F+/ | F+/ | F−/ | F−/ | F+/ | F+/ |
| ID | Cells | Tha- | Tha+ | Tha+ | Tha− | Tha− | Tha+ | Tha+ | Tha− |
| 1 | 13.3 | 52.1 | 53.4 | 62.5 | 62 | 52.1 | 48.9 | 62.5 | 62 |
| 2 | 13.6 | 50.5 | 36.7 | 53.5 | 63.5 | 47.6 | 36.7 | 53.1 | 59.7 |
| 3 | 15.0 | 42.7 | 42 | 53.6 | 58.4 | 42.7 | 41.7 | 53.6 | 57.8 |
| Avg | 14.0 | 48.4 ± 5.0 | 44.0 ± 8.5 | 56.5 ± 5.2 | 61.3 ± 2.6 | 47.5 ± 4.7 | 42.4 ± 6.1 | 56.4 ± 5.3 | 59.8 ± 2.1 |

Additional population analysis comparisons of early apoptotic cell populations (batches of cells) prepared with and without anti-coagulant added, show the consistency of these results.

TABLE 7
Cell population analysis comparison between batches prepared with and
without anticoagulant

| | | | | | | ApoCell | |
|---|---|---|---|---|---|---|---|
| | | At | | ApoCell | | Time 24 h | |
| | | Thawing | | Time 0 h | | Storage | |
| | | w\o | | w\o | | w\o | |
| Test | Specification | ACDhep | +ACDhep | ACDhep | +ACDhep | ACDhep | +ACDhep |
| Change in | >35.0% | 85.5 | 82.8 | 49.9 | 66.7 | 49.0 | 66.7 |
| Total Cell | | (79.5-92.5) | (67.7-96.4) | (46.6-52.3) | (62.5-71.2) | 46.6-50.3) | (62.5-71.2) |
| Count | | | | | | | |
| Percent | | | | | | | |
| change | | | | | | | |
| (min-max) | | | | | | | |
| Changes in | 90.0 ± 10.0% | | | 100 | 100 | 98.2 | 100 |
| ApoCell | | | | | | (96.2-100) | |
| Percent | | | | | | | |
| change | | | | | | | |
| Range (min- | | | | | | | |
| max) | | | | | | | |
| Cell | >85.0% | 98.0 | 96.0 | 98.5 | 94.6 | 97.7 | 94.5 |
| viability PI | | (97.4-98.4) | (91.9-98.1) | (97.9-99.2) | (93.5-95.5) | (96.4-98.6) | (93.4-95.1) |
| exclusion | | | | | | | |
| Percent | | | | | | | |
| viable | | | | | | | |
| Range (min- | | | | | | | |
| max) | | | | | | | |
| Identity/ | CD3 (T | 75.7 | 66.5 | 73.3 | 62.8 | 71.6 | 64.2 |
| Purity | cells): | (71.6-81.4) | (60.1-70.1) | (70.3-78.3) | (61.1-65.3) | (61.5-79.1) | (61.6-68.1) |
| Analysis of | 71.9 | | | | | | |
| cell | (50.0-85.0) | | | | | | |
| phenotype | | | | | | | |
| Average | | | | | | | |
| (%) | ApoCell | | | | | | |
| (maximal | CD3: | | | | | | |
| calculated | 71.6 | | | | | | |
| range) | (50.0-85.0) | | | | | | |
| | CD19 (B | 7.5 | 9.8 | 9.0 | 9.9 | 9.5 | 9.7 |
| | cells): | (4.0-11.1) | (8.6-12.0) | (7.6-10.2) | (9.3-10.2) | (8.6-10.3) | (9.2-10.4) |
| | 9.3 | | | | | | |
| | (3.0-15.0) | | | | | | |
| | ApoCell | | | | | | |
| | CD19: | | | | | | |
| | 9.5 | | | | | | |
| | (4-15) | | | | | | |
| | CD14 | 9.8 | 14.0 | 11.6 | 15.4 | 9.3 | 16.1 |
| | (monocytes): | (6.4-13.0) | (8.8-22.1) | (10.2-13.3) | (8.2-19.3) | (4.8-17.2) | (9.0-20.4) |
| | 10.1 | | | | | | |
| | (2.5-22.0) | | | | | | |
| | ApoCell | | | | | | |
| | CD14: | | | | | | |
| | 10.6 | | | | | | |
| | (2.5-22.0) | | | | | | |
| | CD15^{high} | 0.2 | 0.46 | 0.2 | 0.083 | 0.1 | 0.09 |
| | (granulocytes): | (0-0.3) | (0.18-0.69) | (0.1-0.4) | (0.08-0.09) | (0.1-0.2) | (0.07-0.1) |
| | 0.4 | | | | | | |
| | (0-6.0) | | | | | | |
| | ApoCell | | | | | | |
| | CD15^{high}: | | | | | | |
| | 0.2 | | | | | | |
| | (0-2.0) | | | | | | |
| | CD 56 | 7.4 | 10.1 | 4.7 | 11.2 | 4.9 | 10.0 |
| | (NK): | (2.4-11.0) | (6.6-14.2) | (2.7-8.0) | (7.2-14.2) | (2.2-9.2) | (6.4-13.0) |
| | 7.2 | | | | | | |
| | (1.5-22.0) | | | | | | |
| | ApoCell | | | | | | |
| | CD56: | | | | | | |
| | 5.2 | | | | | | |
| | (1.5-15.0) | | | | | | |

Percentage of final product cells (yield) in the presence or absence of anticoagulants. Similar to the results presented above at Table 3, the data presented in Table 6 demonstrates that early apoptotic cells manufactured from cells frozen in the presence of anticoagulant had a beneficial effect on average yield of fresh final product (FP to) as compared to cells frozen without anticoagulant. The beneficial effect was seen when anticoagulant was used while freezing only (61.3±2.6% versus 48.4±5.0%), or both freezing and thawing (56.5±5.2% versus 48.4±5.0%). The beneficial effect was less significant when anticoagulant was used upon thawing only (44.0±8.5% versus 48.4±5.0%). These were non-high triglyceride samples.

Effect of anticoagulants on aggregation. No cell aggregations were seen in these 3 non-high triglyceride samples, or in 21 additional samples (data not shown). However, in 41 other non-high triglyceride samples manufactured without anticoagulants (data not shown), mild aggregates were seen in 10 (24.4%) and severe aggregates in 5 (12.2%); thus, anticoagulants avoid completely cell aggregates.

Effect of anticoagulants on stability. Fresh FPs manufactured with- or without anticoagulants were stored at 2-8° C. for 24 hours to determine whether addition of ACDhep to the manufacturing procedure impairs the stability of the FP. Cells were sampled following 24 hours of storage and yield was calculated In cell count. Similar to the results shown in Table 3 for extended time periods (up to 72 hours), Table 6 shows that the beneficial effect was kept and observed when anticoagulant was used while freezing only (59.8±2.1% versus 47.5±4.7%), or both freezing and thawing (56.4±5.3% versus 47.5±4.7%). The beneficial effect was less significant when anticoagulant was added only upon thawing (42.4±6.1% versus 47.5±4.7%). These were all non-high triglyceride samples. These results show minimal cell loss following 24 hours of FP storage in all treatments with significant advantage to cells treated with anticoagulant during both freezing and thawing. Average loss of cells treated with anticoagulant during freezing only was 2.3±3.2% compared to 1.9±3.3% without anticoagulants, upon thawing only was 3.0±4.7 compared to 1.9±3.3% without anticoagulants, and 0.2±0.4% compared to 1.9±3.3% without anticoagulants when cells were both frozen and thawed with ACDhep. In summary, the beneficial effect of anticoagulants on yield was kept for at least 24 hours.

The characteristics of a representative cell population of the FP are shown below in Table 8.

TABLE 8
Characterization of the cell population of fresh (t0) FP manufactured from cells
collected with (“+”) or without (“−”) addition of anticoagulant
during freezing (“F”) and thawing (“Tha”) procedures.*

| | FP t0 | | | | | | | | | |
|---|---|---|---|---|---|---|---|---|---|---|
| | F−/Tha− | | | | | F−/Tha+ | | | | |
| Donor | CD3+ | CD19+ | CD56+ | CD14+ | CD15+ | CD3+ | CD19+ | CD56+ | CD14+ | CD15+ |
| ID | (%) | (%) | (%) | (%) | (%) | (%) | (%) | (%) | (%) | (%) |
| 1-3 | 62.2 ± 6.1 | 5.6 ± 0.7 | 9.8 ± 0.9 | 13.5 ± 1.1 | 0 ± 0 | 61 ± 6.1 | 8.6 ± 0.4 | 86 ± 0.9 | 14.1 ± 1.1 | 0 ± 0 |
| | FP t0 | | | | | | | | | |
| | F+/Tha+ | | | | | F+/Tha− | | | | |
| Donor | CD3+ | CD19+ | CD56+ | CD14+ | CD15+ | CD3+ | CD19+ | CD56+ | CD14+ | CD15+ |
| ID | (%) | (%) | (%) | (%) | (%) | (%) | (%) | (%) | (%) | (%) |
| 1-3 | 63.9 ± 5.8 | 7.4 ± 0.6 | 9.4 ± 0.8 | 13.3 ± 1.90 ± 0 | 61.9 ± 6.0 | 11.5 ± 1.1 | 10.1 ± 1.0 | 14.3 ± 1.3 | 14 | 0 ± 0 |

*Induction of apoptosis was performed using a medium containing anticoagulant for all batches.

The results of Table 8 show the cell characteristics of the final products (FP) manufactured with or without anticoagulant at freezing and thawing. Batches were sampled, stained for mononuclear markers, and analyzed via flow cytometry to determine the cell distribution in each sample and to examine whether the addition of anticoagulant affected the cell population. As presented in Table 7, there were no significant differences detected in cell populations manufactured with or without anticoagulants at freezing or thawing. The average T cell population (CD3+ cells) in fresh FP was 62.3±1.2% between treatments compared to 62.9±1.1% before freezing; the average B cell population (CD19+ cells) was 8.3±2.5% between treatments compared to 3.1±0.8% before freezing; the average natural killer cell population (CD56+ cells) was 9.5±0.7% between treatments compared to 12.9±0.5% before freezing; the average monocyte cell population (CD14+ cells) was 13.8±0.5% between treatments compared to 17.5±0.3% before freezing; and the average granulocyte population (CD15+ cells) was 0.0% in the fresh FP compared to 0.35±0.2% at freezing.

The potency of the early apoptotic population was also examined.

TABLE 9
Potency analysis of fresh (t0) FP manufactured from cells with (“+”) or without (“−”)
addition of anticoagulant during freezing (“F”) and thawing (“Tha”) procedures.

| | Donor ID # | | | | | | | |
|---|---|---|---|---|---|---|---|---|
| | FP t0 | | | | | | | |
| Treatment | F−/Tha− | | F−/Tha+ | | F+/Tha+ | | F+/Tha− | |
| Median | DR | CD86 | DR | CD86 | DR | CD86 | DR | CD86 |
| fluorescence | | | | | | | | |
| DCs 1:2 Early | 3% | 28% | 4% | 24% | 5% | 24% | 9% | 15% |
| apoptotic cell | | | up | | | | | |
| population + | | | from | | | | | |
| LPS | | | LPS | | | | | |
| DCs 1:4 Early | 4% | 38% | 6% | 35% | 6% | 34% | 6% | 24% |
| apoptotic cell | | | | | | | | |
| population + | | | | | | | | |
| LPS | | | | | | | | |
| DCs 1:8 Early | 13% | Not | 10% | 45% | 15% | 54% | 8% | 48% |
| apoptotic cell | | done | | | | | | |
| population + | | | | | | | | |
| LPS | | | | | | | | |

The results presented in Table 9 are from a potency assay performed to determine the ability of each final product to enhance a tolerogenic state in immature dendritic cells (iDCs) following stimulation with (LPS). The tolerogenic effect was determined by assessing downregulation of co-stimulatory molecule HLA-DR and CD86 expression on iDCs following interaction with the early apoptotic cell populations and different treatments leading to LPS upregulation. The analysis was performed on DCsign+ cells. Results represent the percent delay in maturation following interaction with early apoptotic cell population and following addition of LPS versus LPS-induced maturation. The experiment tested the potency of fresh FP (t0) manufactured with- or without anticoagulant. Results presented in Table 9 show that apoptotic cells manufactured with or without anticoagulant enhance the tolerance effect of both co-stimulatory markers in a dose-dependent manner.

The early apoptotic cells produced herein were from non-high triglyceride samples. This consistent high yield of stable early apoptotic cells was produced even in the cases when the donor plasma is high in triglycerides (See for example, Examples 12 and 13 of International Publication No. WO 2014/087408 and United States Application Publication No. US US-2015-0275175-A1). Note that anti-coagulants were not added to the PBS media used for formulation of the final early apoptotic cell dose for infusion.

Summary

The objective of this study was to produce a stable, high yield early apoptotic cell population. The rational for use of anticoagulants was that aggregates were seen first in patients with high triglycerides, but later in a significant portion of other patients. A concern here was the disclosure in U.S. Pat. No. 6,489,311 that the use of anticoagulants prevented cell apoptosis.

In short, with minimal impact on the composition, viability, stability, and the apoptotic nature of the cells, there was a significant improvement of at least 10-20% in the number of collected cells in the final product (Yield) when anticoagulant was added. In this study an up to 13% increase in yield was shown, which represents 26.8% augmentation in yield in controlled conditions but in real GMP conditions it went up to 33% and more augmentations in cell number then can be produced in a single collection. This effect is crucial, since it may avoid the need for a second apheresis from a donor.

This effect was surprising because the anticipated impact was expected to be dissolution of mild aggregates. It had been hypothesized that thawing cells with anticoagulant reduced the number of aggregates. When formed, these aggregates eventually lead to massive cell loss. Cells collected and frozen without anticoagulant demonstrated aggregate formation at thawing, immediately after wash. Furthermore, a high level of aggregates was also detected in cells that were frozen without anticoagulant and resuspended with media containing anticoagulant. No aggregates were seen in cells that were both frozen and resuspended with media containing anticoagulant. Taken together, it was concluded that the addition of anticoagulants during freezing and apoptosis induction is of high importance, and did not appear to negatively impact the induction of early apoptosis on the cell population.

Recovery of early apoptotic cells was further tested, for example, following 24 hours of storage at 2-8° C., for stability purposes, during which an average cell loss of 3-4.7% was measured, regardless of manufacturing conditions, with favorable results for cells that were both frozen and thawed with media containing anticoagulant (0.2±0.4% cell loss following 24 hours of FP storage), suggesting that addition of anticoagulant is critical during freezing and thawing, but once finally formulated, the early apoptotic cell population is stable. Extended time point studies showed this stability to at least 72 hours.

Apoptosis and viability, as well as cell composition of the FP product were not significantly affected by the addition of anticoagulant at the freezing and/or thawing stage. Values measured from a wide variety of characteristics were similar, indicating the ACDhep did not change the early apoptotic cell characteristics and the final product met the acceptance criteria of ≥40% apoptotic cells.

The assay used to test apoptotic cells potency was based on immature dendritic cells (iDCs), DCs that are characterized by functions such as phagocytosis, antigen presentation, and cytokine production.

The HLA-DR (MHC class II) membrane molecule and co-stimulatory molecule CD86 were selected as markers to detect the tolerogenic effects of antigen-presenting cells (APCs). Using flow cytometry, changes in expression of HLA-DR and CD86 on iDCs were measured following stimulation with LPS, as well as in the presence of the early apoptotic cell population manufactured with- or without anticoagulant and stimulated with LPS. Early apoptotic cell populations were offered to DCs in ascending ratios of 1:2, 1:4, and 1:8 iDCs:early apoptotic cell population. As presented in Table 6, it was shown that early apoptotic cell population enhanced the tolerogenic effect over stimulated DCs in a dose-dependent manner, with slightly better results for early apoptotic cell population manufactured with anticoagulant both at freezing and apoptosis induction.

Taken together, it was concluded that addition of anticoagulant to both freezing and apoptosis media is of high importance to increase cell recovery and avoid massive cell loss due to aggregates, and to avoid in many cases a second round of apheresis from a donor. It was shown that all cells met acceptance criteria for the validated FP, indicating that the addition of anticoagulant does not impair the FP.

### Example 2: Stability Criteria for Apoptotic Cells from Multiple Individual Donors

The objective of this study is to develop stability criteria for apoptotic cells from multiple individual donors with comparability studies to non-irradiated HLA-matched apoptotic cells (Mevorach et al. (2014) Biology of Blood and Marrow Transplantation 20(1): 58-65; Mevorach et al. (2015) Biology of Blood and Marrow Transplantation 21(2): S339-S340).

Apoptotic cell final product preparations will be evaluated for cell number, viability, early apoptotic phenotype and potency after storage at 2 to 8° C. for 8, 24, 48, and 60 hours with sampling at each time point. Apoptotic cell final product lots will be prepared following standard operating procedures (SOPs) (Example 1) and batch records (BRs; i.e., specific manufacturing procedures). For potency evaluation, samples of early apoptotic cell preparation final product lots will be tested for inhibition of lipopolysaccharide (LPS) induced upregulation of MHC-II expression on immature dendritic cells (time points 0-24 h) or monocytes (time points 0-6) and will be performed according to SOPs and recorded on BR. These series of test will be performed on pooled and non-pooled products that are in preparations originating from multiple individual donors and from single donors, respectively.

In addition, flow cytometric analysis of CD3 (T cells), CD19 (B cells), CD14 (monocytes), CD15^{high} (granulocytes) and CD56 (NK cells) will be documented. The aims of these studies are to demonstrate consistency with a narrow range of results. Preliminary results are consistent with these goals and no deviations from the SOP are noted and no technical problems are reported. However, further studies are needed in order to conclude the range and stability of effective treatment. Preliminary results show equivalence in all these parameters. Further, single donor stability studies showed stability at least through a 48-hour period (See, Example 1).

### Example 3: Effect of Irradiation on Final Apoptotic Cell Product

Apoptotic cells are increasingly used in novel therapeutic strategies because of their intrinsic immunomodulatory and anti-inflammatory properties. Early apoptotic cell preparations may contain as much as 20-40% viable cells (as measured by lack of PS exposure and no PI admission; Annexin V negative and Propidium iodide negative) of which some may be rendered apoptotic after use in a transfusion, but some will remain viable. In the case of bone marrow transplantation from a matched donor, the viable cells do not represent a clinical issue as the recipient is already receiving many more viable cells in the actual transplant. However, in the case of a third-party transfusion, (or fourth party or more as may be represented in a pooled mononuclear apoptotic cell preparation) use of an apoptotic cell population that includes viable cells may introduce a second GvHD inducer. Furthermore, the implication of irradiation on the immunomodulatory potential of early apoptotic cells has so far been not assessed. A skilled artisan may consider that additional irradiation of an early apoptotic cell population may lead cells to progress into later stages of apoptosis or necrosis. As this appears a particularly relevant question with regard to clinical applications, the experiments presented below were designed to address this issue, with at least one goal being to improve the biosafety of functional apoptotic cells.

Thus, the aim was to facilitate the clinical utilization of apoptotic cells for many indications wherein the potency of apoptotic cells may rely on a bystander effect rather than engraftment of the transplanted cells.

Objective: Examine the effect of irradiation on early apoptotic cells, wherein irradiation occurs following induction of apoptosis.

Methods (in brief): Three separate early apoptotic cell batches were prepared on different dates (collections 404-1, 0044-1 and 0043-1).

Each final product was divided into three groups:

Untreated

2500 rad

4000 rad.

Following irradiation, early apoptotic cells were tested immediately (to) for cell count, Annexin V positive-PI negative staining, cell surface markers (% population of different cell types) and potency (dendritic cells (DCs)). Following examination at to, early apoptotic cells were stored at 2-8° C. for 24 hours, and examined the next day using the same test panel (t_{24 h}) (cell count, Annexin V positive-PI negative staining, and cell surface markers and potency).

Previously, a post-release potency assay was developed, which assesses the ability of donor mononuclear early apoptotic cells (Early apoptotic Cells) to induce tolerance (Mevorach et al, BBMT 2014 ibid). The assay is based on using flow cytometric evaluation of MHC-class II molecules (HLA-DR) and costimulatory molecule (CD86) expression on iDC membranes after exposure to LPS. As previously and repeatedly shown, tolerogenic DCs can be generated upon interaction with apoptotic cells (Verbovetsky et al., J Exp Med 2002, Krispin et al., Blood 2006), and inhibition of maturation of LPS-treated DCs (inhibition of DR and CD86 expression), occurs in a dose dependent manner.

During phase 1/2a of the early apoptotic cell clinical study, the post-release potency assay was conducted for each early apoptotic cell batch (overall results n=13) in order to evaluate the ability of each batch to induce tolerance (Results are shown in FIG. 1, Mevorach et al. (2014) Biology of Blood and Marrow Transplantation 20(1): 58-65).

DCs were generated for each early apoptotic cell batch from fresh buffy coat, collected from an unknown and unrelated healthy donor, and were combined with early apoptotic cells at different ratios (1:2, 1:4 and 1:8 DC:Early apoptotic Cells, respectively). After incubation with early apoptotic cells and exposure to LPS, potency was determined based on downregulation of DC membrane expression of either HLA DR or CD86 at one or more ratios of DC:early apoptotic cells. In all 13 assays, early apoptotic cells demonstrated a tolerogenic effect, which was seen with preparations at most DC:early apoptotic cells ratios, and for both markers, in a dose dependent manner.

Monocyte obtained immature DCs (iDCs) were generated from peripheral blood PBMCs of healthy donors and cultured in the presence of 1% autologous plasma, G-CSF and IL-4. iDCs were then pre-incubated for 2 hours at 1;2, 1;4 and 1;8 ratios with apoptotic cells either freshly prepared final product or final product stored at 2-8° C. for 24 hours. The two final products were examined simultaneously in order to determine whether storage affects potency ability of apoptotic cells. Following incubation, LPS was added to designated wells were left for additional 24 hours. At the end of incubation, iDCS were collected, washed and stained with both DC-sign and HLA-DR or CD86 in order to determine changes in expression. Cells were analyzed using flow cytometer and analysis performed using FCS-express software from DC-sign positive cells gate to assure analysis on DCs only.

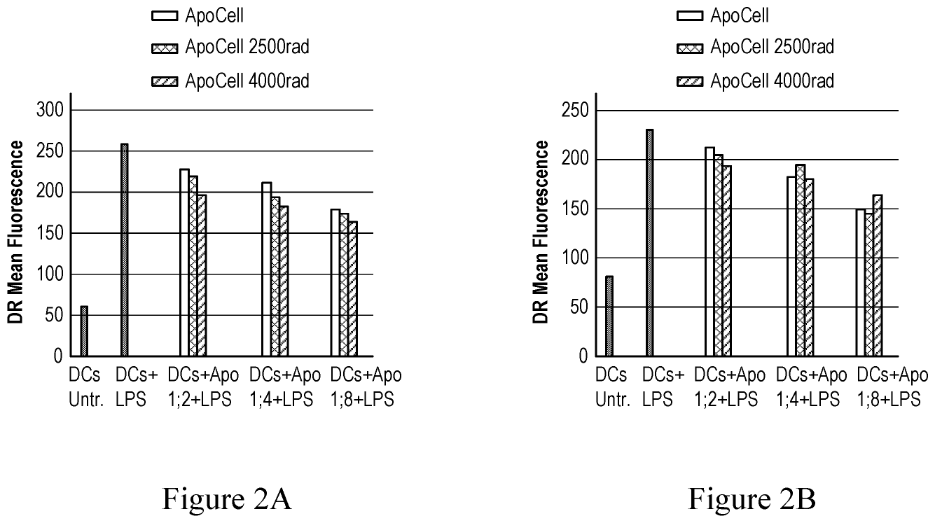

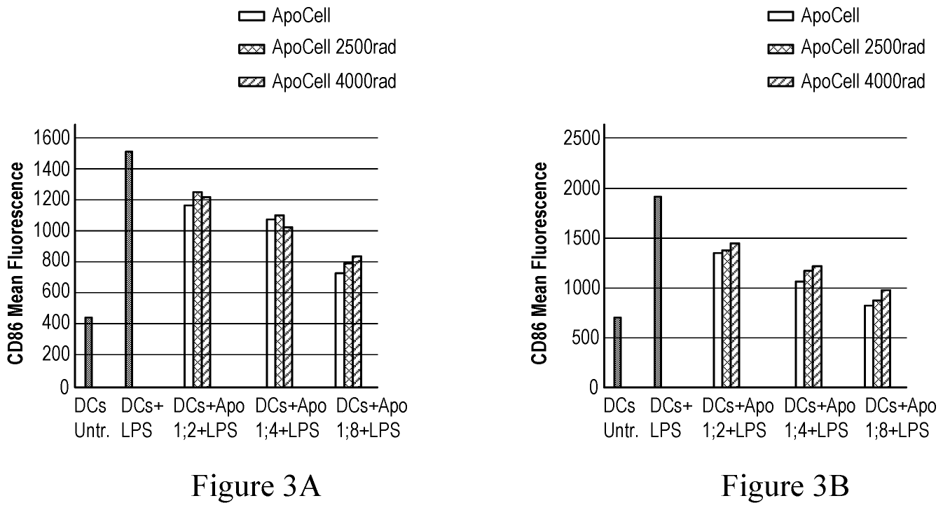

FIGS. 2A and 2B and FIGS. 3A and 3B show potency test of irradiated pooled apoptotic cells compared to non-irradiated single donor cell.

Results:

Single Donor Preparations

Table 10 presents the comparative results of non-radiated and irradiated apoptotic cells; Average cell loss (%) at 24 hours; Annexin positive(⁺) Propidium Iodide (PI) negative(⁻) % at 0 hours and 24 hrs (% of early apoptotic cells; Annexin positive (⁺) Propidium Iodide (PI) positive (⁺) % at 0 hours and 24 hrs (% of late apoptotic cells); presence of cell surface antigens CD3 (T cells), CD19 (B cells), CD56 (NK cells), CD14 (monocytes), and CD15^{high} (granulocyte), at 0 hours and 24 hours.

TABLE 10

| Final product | Apoptotic | Apoptotic Cell | Apoptotic Cell |
|---|---|---|---|
| description | Cell | 2500rad | 4000rad |
| An⁺PI⁻ t₀ (%) | 59.2 | 59.6 | 58.4 |
| Range (min-max) | (52.6-66.1) | (51.6-68.7) | (50.4-65.1) |
| An⁺PI⁻ t_{24 h} (%) | 62.6 | 68.1 | 66.7 |
| Range (min-max) | (53.6-76.3) | (52.0-81.3) | (52.9-77.1) |
| An⁺PI⁺ t₀ (%) | 4.9 | 6.0 | 6.1 |
| Range (min-max) | (3.2-7.0) | (5.2-7.4) | (4.0-9.1) |
| An⁺PI⁺ t_{24 h} (%) | 7.3 | 8.6 | 9.0 |
| Range (min-max) | (5.0-11.8) | (6.4-11.8) | (6.0-14.9) |
| CD3+ t₀ (%) | 56.9 | 58.3 | 57.5 |
| Range (min-max) | (47.4-66.3) | (48.8-67.7) | (48.6-66.4) |
| CD3+ t_{24 h} (%) | 56.8 | 57.1 | 56.6 |
| Range (min-max) | (49.6-64.0) | (48.0-66.1) | (49.7-63.4) |
| CD19+ t₀ (%) | 10.6 | 9.5 | 9.6 |
| Range (min-max) | (10.1-11.0) | (7.7-11.3) | (8.5-10.7) |
| CD19+ t_{24 h} (%) | 11.8 | 9.2 | 8.8 |
| Range (min-max) | (10.2-13.4) | (6.9-11.5) | (7.5-10.1) |
| CD56+ t₀ (%) | 12.2 | 13.0 | 14.4 |
| Range (min-max) | (7.0-17.3) | (7.6-18.4) | (21.2-7.6) |
| CD56+ t_{24 h} (%) | 12.9 | 14.1 | 17.1 |
| Range (min-max) | (8.8-13.4) | (10.4-17.8) | (10.0-24.1) |
| CD14+ t₀ (%) | 23.1 | 25.2 | 24.6 |
| Range (min-max) | (13.1-33.1) | (13.8-36.5) | (14.0-35.2) |
| CD14+ t_{24 h} (%) | 21.9 | 23.7 | 24.4 |
| Range (min-max) | (13.8-30.0) | (13.8-33.6) | (15.4-33.4) |
| CD15^{high} t₀ (%) | 0.0 | 0.0 | 0.01 |
| Range (min-max) | | | (0.0-0.02) |
| CD15^{high} t_{24 h}(%) | 0.0 | 0.0 | 0.01 |
| Range (min-max) | | | (0.0-0.02) |

The results in Table 10 show that both non-irradiated apoptotic cells and irradiated apoptotic cells had comparable percentages of early (rows 2 and 3) and late (rows 4 and 5) apoptotic cells. Thus, 25 or 40 Gy irradiation did not accelerate the apoptotic or necrotic process induced prior to this high level of gamma-irradiation. Further, there was consistency between irradiated cell populations vs. control non-irradiated population with regard to cell type.

The results of potency assays, presented in FIGS. 2A-2B (HLA-DR expression) and FIGS. 3A-3B (CD86 expression) show that there was no change in the immune modulatory capacity of fresh (FIG. 2A, FIG. 3A) and 24 hour-stored (FIG. 2B and FIG. 3B) irradiate apoptotic cells when compared with non-irradiated apoptotic cells.

In both FIGS. 2A-2B and FIGS. 3A-3B there is a clear upregulation in both HLA-DR and CD86 expression, following exposure to maturation agent LPS. Significant (p<0.01), dose-dependent down regulation of both co-stimulatory markers was observed in the presence of freshly prepared apoptotic cells both from a single donor or irradiated pooled donors. In addition, dose dependent down regulation was maintained in both markers in the presence of apoptotic cells stored at 2-8° C. for 24 hours, indicating final product stability and potency following 24 hours of storage.

Effect on dendritic cells. In order to test the immunomodulatory capacity of apoptotic cells a post release potency assay was used (Mevorach et al., (2014) BBMT, ibid). No change in immune modulatory assay in dendritic cells was observed. (Data not shown)

Effect on Mixed Lymphocyte Reaction (MLR). In order to further test the immunomodulatory effect a standardized MLR assay was established. Here, co-cultivation of stimulator and responder cells, i.e., a MLR, yielded strong and reliable proliferation. Upon addition of non-irradiated apoptotic cells to the MLR, the lymphocyte proliferation was significantly reduced by >5-fold, clearly demonstrating cell inhibition of proliferation. Inhibition of lymphocyte proliferation in MLRs mediated by irradiated apoptotic cells was completely comparable. (Data not shown)

The next step was to evaluate in vivo, irradiated and non-irradiated apoptotic cells in a completely mismatched mouse model. As shown, irradiated and non-irradiated early apoptotic cell preparations had comparable in vivo beneficial effect.

Single Donor Preparations Conclusion:

In conclusion, irradiation of 25 Gy or 40 Gy did not significantly accelerate apoptosis or induced necrosis in populations of apoptotic cells. Significantly, these populations maintained the immunomodulatory effect of apoptotic cells both in vitro and in vivo.

Multiple Donor Preparations

Next, experiments were performed to verify that the phenomenon observed with single donor, third party preparation was also true for multiple third-party donors. Unexpectedly, when using pooled individual donor apoptotic cell preparations, the beneficial effect of a single unmatched donor was lost. This was not due to GvHD, as the beneficial effect of each donor separately was maintained (test results no shown). One possibility is that the beneficial effect of the early apoptotic cell preparation was lost due to the interaction of the individual donor cells among themselves. It was further examined whether this possible interaction of different donors could be avoided by gamma irradiation.

As shown, the beneficial effect of a single donor was completely restored following gamma irradiation, wherein the irradiated multiple donor preparation and the single donor preparation (irradiated or non-irradiated) had similar survival patterns.

Conclusion:

It is shown here for the first time that surprisingly irradiation (and possibly any method leading to T-cell Receptor inhibition) not only avoided unwanted proliferation and activation of T-cells but also allowed for the beneficial effects of immune modulation when using a preparation of multiple donor third party apoptotic cells.

### Example 4: Prevention of SARS2-CoV2 Corona-Virus-Related Organ Failure

Asymptomatic subjects exposed to SARS2-CoV2 corona virus are treated by the early apoptosis compositions disclosed herein. Then, their overall health, including cytokine/chemokine levels and key organ function is monitored for at least about three weeks in search of rise in body temperature or any other clinical indication or symptom associated with SARS2-CoV2 corona virus infection. The function of key organs is also monitored according to standard protocols. Subjects may be model animal subjects, such as ferrets or bats.

### Example 5: Treatment of SARS2-CoV2 Corona-Virus-Related Organ Failure

Symptomatic subjects exposed to SARS2-CoV2 corona virus are treated by the early apoptosis compositions disclosed herein. Then, their overall health, including cytokine/chemokine levels and key organ function is monitored for at least about three weeks is search of rise in body temperature or any other clinical indication or symptom associated with Corona virus infection.

### Example 6: A Multi-Center Open Label Study, Evaluating Safety of Allocetra-OTS for the Prevention of Organ-Failure Deterioration in Severe Patients with COVID19 and Respiratory Dysfunction

Objective: To assess the use of early apoptotic cells (Allocetra) in combination with standard of care therapy in patients with COVID-19, which is some cases was associated lung dysfunction. Evaluation includes safety, tolerability, cytokine profile, and efficacy parameters, wherein changes in PaO₂/FiO₂ ratio number, and severity of adverse events and serious adverse events serve as the co-primary study endpoints.

Preliminary Efficacy: To assess prevention of respiratory deterioration associated with COVID-19.

Primary end point: To evaluate safety of Allocetra-OTS in subjects with respiratory dysfunction and COVID19.

Methods:

Study Design—This is a multi-center, open-label study evaluating safety of Allocetra-OTS, in subjects with severe or critical COVID19 and respiratory dysfunction.

A preliminary clinical study was performed wherein five (5) COVID-19 patients were selected, and identified as suffering from severe (3 subjects) or critical (2 subject) COVID19. The two (2) critical subjects were not on a respirator. Thus, the patients enrolled in the study included those with COVID-19 in difficult and serious condition.

After signing an informed consent by the patient and within 24+6 hours following the time of eligibility (time 0), on Day 1, eligible recipient subjects received single intravenous (IV) administration of investigational product (IP) as described below. Administration included a composition comprising Allocetra-OTS treatment (unmatched early apoptotic mononuclear cells from a foreign donor) at 140×10⁶±20% cells/kg body weight (screening body weight) in 375 mL of Ringer's lactate solution. Subjects were followed for efficacy and safety assessments over time, for example 28 days following investigational product administration. Further, changes in PaO₂/FiO₂ ratio will be followed for at least 28 days (See Follow-up in Example 7).

Subjects were hospitalized for COVID 19, and later as medically indicated. Following administration of the Allocetra product by intravenous (IV) injection (Day 1), subjects were followed for efficacy and safety assessments through 28 days. Number of visits for subjects participating in this study was on Days 3, 5, and 7. The larger multi-center study will include visits at days 14 and 28, as well.

Study Duration—For each participating subject, the duration in the study was up to 28 days as follows:

TABLE 11
Study Duration

| Study Periods | Duration |
|---|---|
| Screening (Day-1) | Up to 1 day |
| Treatment Day (Day 1) | 1 day |
| Short term follow-up (Day 2 to Day 7 | 6 days |
| (inclusive)) | |
| Medium term follow-up (Day 8 to Day 14 | 7 days |
| (inclusive)) | |
| Long term follow-up (Day 15 to Day 28) | 14 days |

Eligibility Criteria—male or female >18 and <80-year-old diagnosed with respiratory dysfunction and COVID19, as defined below:
Laboratory confirmation of SARS-COV2 infection by reverse-transcription polymerase chain reaction (RT-PCR) from any diagnostic sampling source.
Patients classified as severe or critical according to NIH severity classification (See below).
The 5 patients in the clinical study were additionally treated by the treating physician with Clexane at a minimal dose of 40 mg a day, dexamethasone, and for some remdesivir.

Exclusion Criteria—
Pregnancy, lactation and childbearing potential woman who are not willing to use acceptable contraceptives measures for the entire study duration.
Combined with other organ failure (need organ support not including respirator)) including Stage 4 severe chronic kidney disease or requiring dialysis (i.e. estimated glomerular filtration rate (eGFR)<30).
Patients with malignant tumor, other serious systemic diseases and psychosis.
Patients who are participating in other clinical trials or treated with any experimental agents that may contradict this trial (i.e, biologics)
Co-Infection of HIV, tuberculosis.
Known immunocompromised state or medications known to be immunosuppressive (see concomitant prohibited medications on the next page).
Intubated patients (due to inability to sign an informed consent)
Patients with P/F ratio of <150 or a change in status of eligibility manifested by a rapid decline of P/F ratio between eligibility status and actual drug delivery.

Study intervention, Route of Administration and Dosage Form

Investigational Product (IP): Allocetra-OTS is a cell-based therapeutic composed of donor early apoptotic cells. Early apoptotic cells were prepared as per Example 1 above. The product contained allogeneic donor mononuclear enriched cells (unmatched cells from a foreign donor) in the form of a liquid suspension with at least 40% early apoptotic cells (Annexin V≥40% and PI≤15%). The suspension was prepared with Ringer's lactate solution and administered by IV. The suspension was stored at 2-8° C. until 20+25 minutes before infusion and at room temperature thereafter.

Allocetra-OTS dose contained 140×10⁶±20% cells per kg of recipient body weight (at screening) in a total volume of 375 mL Ringer's lactate solution in a transfer pack that underwent irradiation and was administered by an intravenous route via adjusted filter and using a volumetric pump, at a starting rate of 48 mL/hour (16 drops per minute) with a gradual increase every 15-25 minutes of 15 mL/hour (additional 5 drops per minute) to a maximal rate of 102 mL/hour. Each of the 5 subjects received a single dose of Allocetra-OTS. Additionally, patients received Clexane, dexamethasone, and for some remdesivir.

The study intervention was completed within 72 hours of completing the manufacturing process.

During product administration no other IV fluids such as Ringer's lactate or normal saline was given in parallel, unless medically indicated due to volume depletion.

Patient Classification (NIH:

https://www.covid19treatmentguidelines.nih.gov/overview/management-of-covid-19/):

In general, adults with COVID-19 can be grouped into the following severity of illness categories:
Asymptomatic or Pre-symptomatic Infection: Individuals who test positive for SARS-CoV-2 by virologic testing using a molecular diagnostic (e.g., polymerase chain reaction) or antigen test, but have no symptoms.
Mild Illness: Individuals who have any of the various signs and symptoms of COVID 19 (e.g., fever, cough, sore throat, malaise, headache, muscle pain) without shortness of breath, dyspnea, or abnormal chest imaging.
Moderate Illness: Individuals who have evidence of lower respiratory disease by clinical assessment or imaging and a saturation of oxygen (SpO₂)≥94% on room air at sea level.
Severe Illness: Individuals who have respiratory frequency >30 breaths per minute, SpO₂<94% on room air at sea level, ratio of arterial partial pressure of oxygen to fraction of inspired oxygen (PaO₂/FiO₂)<300 mmHg, or lung infiltrates >50%
Critical Illness: Individuals who have respiratory failure, septic shock, and/or multiple organ dysfunction.

In the clinical study, 2 COVID-19 patients were identified as having critical illness and 3 COVID-19 patients were identified as having severe illness, based on the NIH guidelines provided.

Standard of Care (SOC): The SOC for COVID 19 was according to institutional standards. Institutional SOC may include Clexane, anti-viral agents, chloroquine or hydroxychloroquine, remdesivir or other agents.

Concomitant Medications: Prohibited medications: Significant immune suppressing agents including chronic corticosteroids >10 mg/day, azathioprine, cyclosporine, cyclophosphamide, and any biological treatment. The known SOC medications to treat COVID19; hydroxychloroquine, chloroquine, and azithromycin, are not known to have any possible interaction with Allocetra-OTS. Neither are anti-viral agents.

Handling of blood samples: Blood samples were obtained before investigational product administration (Day 1) and thereafter on day 3, 7, 14, 28, or until release for cytokines/chemokines measurements. Blood samples were obtained and handled according to the institutional guidelines and approval.

Statistical Analysis: The data will be summarized in tables by treatment group over time, listing the mean, standard deviation, minimum, median, maximum and number of subjects for continuous data, or in tables listing count and percentage for categorical and event data, as appropriate. “Time to” data will be described using survival curves. Data listings by subject will be provided.

Descriptive analyses and, where appropriate, statistical testing in the large Open-Label clinical trial will compare between each of the two groups (Allocetra-OTS and vehicle).

All statistical analyses will be performed, and data appendixes will be created using the SAS® system (SAS Institute, Cary, N.C.), Version 9.4 or higher. The effects of noncompliance, dropouts, and possible covariates such as age, gender, and center, will be assessed descriptively to determine the impact on the general applicability of results from this study.

Safety, subject disposition and baseline characteristics will be presented on the safety population. Efficacy will be assessed on FAS and PP populations. A comprehensive description of these analyses will be detailed in the statistical analysis plan (SAP).

Results:

Results showed positive results of a clinical trial of Allocetra™ in COVID-19 patients in severe or critical condition.

Treatment with Allocetra-OTS was safe and all 5 patients responded well. There were no reported severe adverse events relating to the administration of Allocetra™ in the patients, and the therapy was well-tolerated.

All five patients had complete recovery from their respective severe or critical condition and were released from the hospital after an average of 5.5 days (severe) and 8.5 days (critical), following administration of Allocetra™. The average stay for the severe COVID-19 patients treated was 5.5 days and for the critical COVID-19 patients treated it was 8.5 days. Treatment with Allocetra-OTS resulted in all 5 patients being PCR negative for the SARS-CoV-2 virus at the time of release from the hospital. Moreover, there was a dramatic amelioration of O₂ saturation/Flow ratio in the treated patients. Analysis showed that CRP and ferritin levels were reduced.

Analysis of improved respiratory tract dysfunction in a severe COVID-19 patient and improve the PaO₂/FiO₂ ratio in these patients was be performed (See, updated date in Example 7). Patients treated with Allocetra-OTS were released from the hospital healthy and negative for the virus. This included patients in difficult and critical conditions prior to treatment.

Conclusion: At this time and based on the surprising effects of Allocetra-OTS on severe and clinical COVID-19 patients, it was concluded the Allocetra-OTS is safe and should be evaluated in a larger study, especially for use in treating COVID-19 patients in severe or critical COVID-19 condition.

### Example 7: Phase Ib & II Clinical Trials in COVID-19 Patients in Severe or Critical Condition

Objective: To evaluate the safety and efficacy of Allocetra-OTS for the treatment and prevention of organ-failure deterioration in severe and critical patients with COVID19 and respiratory dysfunction.

Methods:

Patients included 5 patients for the evaluation of safety (Phase Ib; This is the same patient population as presented in Example 6. Additional data retrieved from those patients is presented herein.) and up to 24 patients for evaluation both efficacy and safety (Phase II). Inclusion Criteria: (1) Confirmed SARS-CoV-2 infection by PCR and clinical COVID-19, (2) Meets NIH classification for severe or critical disease (See Guidelines above in Example 6), (3) Not on a respirator, and (4) Age 18-82.

The characteristics of the Phase Ib patients are provide in Table 12 below. This is the patient population described and treated in Example 6 above.

TABLE 12
Characteristics of Patients Upon Entry to the Study

| | | | | | | |
|---|---|---|---|---|---|---|
| Patient | 001 | 002 | 003 | 005 | 006 | All |
| Age-years | 44 | 53 | 47 | 59 | 46 | Average 48.5 |
| Gender | F | M | M | F | M | 2 females |
| | | | | | | 3 males |
| Ethnic group | Arab | Arab | Jewish | Arab | Jewish | 3 Arab |
| | | | | | | 2 Jewish |
| Weight (kg.) | 80 | 75 | 108 | 100 | 100 | Average 92.6 |
| Coexisting conditions: | | | | | | |
| Hypertension | No | No | No | Yes | No | 1/5 |
| Diabetes mellitus | No | No | No | Yes | No | 0/5 |
| Chronic kidney disease | No | No | No | No | No | 0/5 |
| Ischemic heart disease | No | No | No | No | No | 0/5 |
| Congestive heart failure | No | No | No | No | No | 0/5 |
| Pregnancy | No | No | No | No | No | 0/5 |
| Overweight (BMI) | Yes (33.3) | No | No (27.8) | Yes (39.1) | Yes | 2/5 |
| Chronic lung disease | No | No | No | No | No | 0/5 |
| Chronic liver disease | No | No | No | No | No | 0/5 |
| Immunosuppression | No | No | No | No | No | 0/5 |
| Malignancy | No | No | No | No | No | 0/5 |

Endpoints for evaluation included safety, time to discharge from the hospital, ventilator and oxygen free days, vasopressor-free days, days with return to basic National Early Warning Score 2 (NEWS2; used to determines the degree of illness of a patient and prompts critical care intervention), time to basic 7 points evaluation ((1) Death; (2) Hospitalized, on invasive mechanical ventilation or extracorporeal membrane oxygenation (ECMO); (3) Hospitalized, on non-invasive ventilation or high flow oxygen devices; (4) Hospitalized, requiring supplemental oxygen; (5) Hospitalized, not requiring supplemental oxygen; (6) Not hospitalized, limitation on activities; (7) Not hospitalized, no limitations on activities), mortality from any cause, cumulative days in the Intensive Care Unit (ICU) or Intensive Management Unit (IMU) and/or in hospital, time to CRP <20 mg/L, and changes in cytokines or chemokine levels for example but not limited to IL-6, IL-18, IFN-α, IFN-γ, IL-10, IL-2Rα, IL-8, and IL-7.

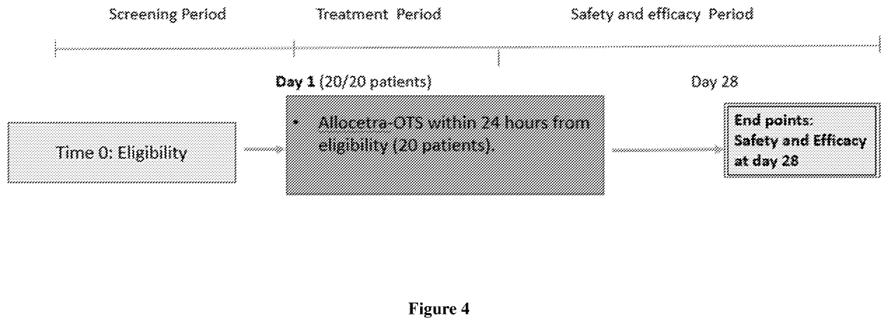

FIG. 4 provides a schematic for the Study.

NIH Guidelines were used to characterize patients. As provided above, patients with severe illness include those individuals who have SpO₂<94% on room air at sea level, a ratio of arterial partial pressure of oxygen to fraction of inspired oxygen (PaO₂/FiO₂)<300 mmHg, respiratory frequency >30 breaths per minute, or lung infiltrates >50%. Patients with critical illness include individuals who have respiratory failure, septic shock, and/or multiple organ dysfunction. (See also Example 6)

Treatment: The patients received a “standard of care” for COVID19 that included both remdesivir (4/5) and dexamethasone (5/5). (See also Example 6)

A single dose of Allocetra-OTS was administered by IV injection. The preparation, dosage, and administration of the Allocetra-OTS were as presented in Example 6 except that in the Phase II trial, a fixed dose of 1×10⁹±20% cells (Allocetra-OTS) was administered again by intravenous (IV) injection.

Results:

Summary Phase I Trial (Initial Results and details presented in Example 6):

Secondary end points. All results of all secondary endpoint are presented individually in Table 13 and further summarized in Table 14.

TABLE 13
COVID-19 Summary of Clinical Characterizations of 5 patients

| | | | | | | |
|---|---|---|---|---|---|---|
| Patient | 001 | 002 | 003 | 005 | 006 | All |
| Covid-19 in real time PCR | Yes | Yes | Yes | Yes | Yes | Yes |
| assay upon presentation | | | | | | |
| NIH severity | Severe | Critical | Critical | Severe | Critical | 2 Severe, 3 |
| classification* | | | | | | Critical |
| Current Treatment: | | | | | | |
| Zinc | − | − | − | − | − | −5/5 |
| Vitamin D | − | − | − | − | − | −5/5 |
| Hydroxychloroquine | − | − | − | − | − | −5/5 |
| Azithromycin | − | − | − | − | − | −5/5 |
| Lopinavir/Ritonavir | − | − | − | − | − | −5/5 |
| Convalescent plasma | − | − | − | − | − | −5/5 |
| Non-specific IVIG | − | − | − | − | − | −5/5 |
| Tocilizumab | − | − | − | − | − | −5/5 |
| Enoxaparin prophylaxis | + | + | + | + | + | +5/5 |
| Remdesivir | No | Yes | Yes | Yes | Yes | +4/5 |
| Favipravir | − | − | − | − | − | −5/5 |
| Dexamethasone | + | + | + | + | + | +5/5 |

TABLE 14
COVID-19 Summary of Clinical Characterizations of 5 patients

| | | | | | | |
|---|---|---|---|---|---|---|
| Patient | 001 | 002 | 003 | 005 | 006 | All |
| Respiratory support | | | | | | |
| category-no. (%) | | | | | | |
| Ambient air | − | − | − | − | − | 0/5 |
| O₂ Nasal Canula | + | − | − | + | − | 2/5 |
| Facial mask/ High | − | + | + | − | + | 3/5 |
| flow oxygen | | | | | | |
| Invasive ventilation | − | − | − | − | − | 0/5 |
| % O₂ Saturation | 89 (RA) | 83 (RA) | 90 (RA) | 90 (RA) | 88 (RA) | 88 (RA) in |
| | 92 (3L; NC) | | 97 (5L; FM) | | | average |
| Lung infiltrates | + | + | + | + | + | |
| Minimal Sat/O₂ | 306 | 121 | 242 | 335 | 268 | 254.4 in |
| Concentration at the | | | | | | average |
| day of Allocetra IV | | | | | | |
| administration | | | | | | |
| ARDS | +Mild | +Moderate | +Moderate | No | +Mild | 4/5 with ARDS |
| | | | | | | 2/5 Moderate |
| | | | | | | 2/5 Mild |
| ICU/Death | − | − | − | − | − | 0/5 |

Three patients with severe COVID-19 (defined as <94% oxygen saturation and the presence of lung infiltrates) and 2 patients with critical COVID-19 (neither was on mechanical ventilation, but both received high flow oxygen and lung infiltrates), were included in this study and were treated.

Following administration of Allocetra, all patients had a favorable outcome, manifested by gradual improvement in respiratory function as shown by gradual improvement in their oxygen saturation/inspired oxygen concentration ratio and clinical signs.

Table 11 above shows patient characteristics including background medical history and concomitant drug administration.

Table 14 above summarizes the clinical course in all 5 patients. On the 7-point severity scale, the initial scores averaged 3.6 and returned to normal (7 points) within 8.8 days for all patients. The NEWS2 was 5 in average and went back to normal (0/1) within 8.8 days. In addition, 4 patients had mild-to-moderate ARDS (2/3 moderate ARDS) and 3 met criteria for critical condition (NIV). All five completely recovered with negative PCR upon discharge. The average hospitalization time was 10.4 days; 6.6 days following administration of Allocetra. No patient needed ICU hospitalization or a respirator even though 4/5 had ARDS. The average stay following Allocetra administration was 3.5 days for patients in severe condition and 8.6 days for patients in critical condition.

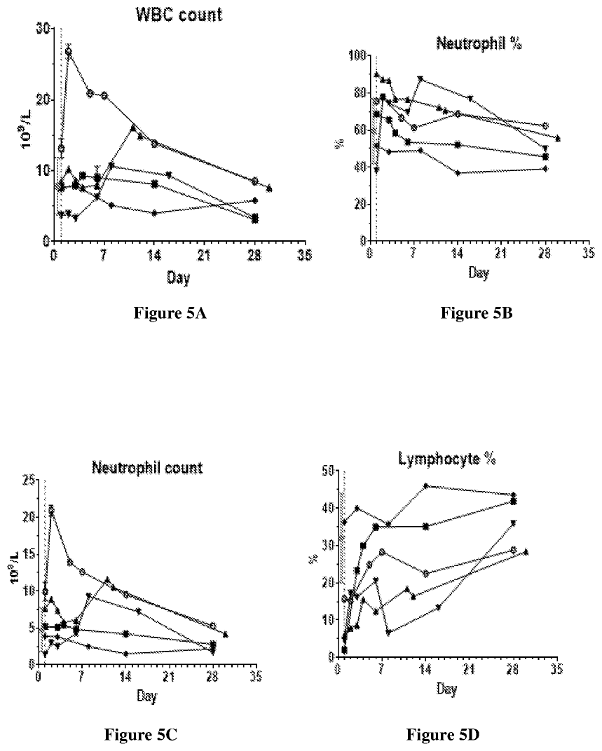

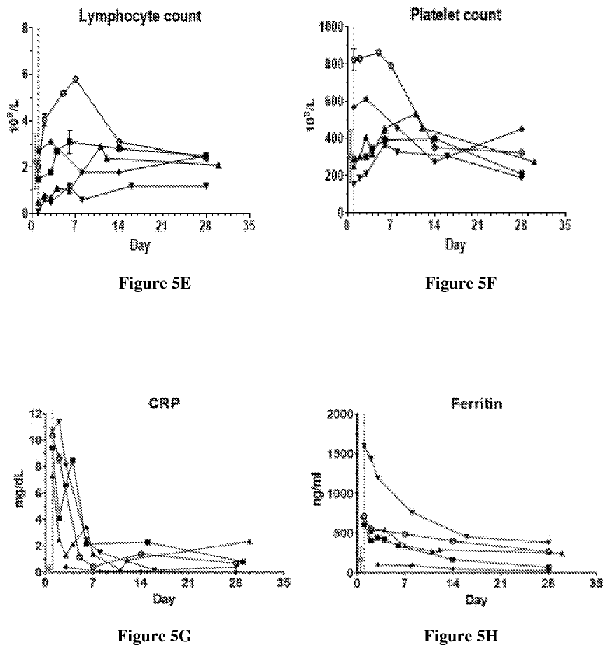

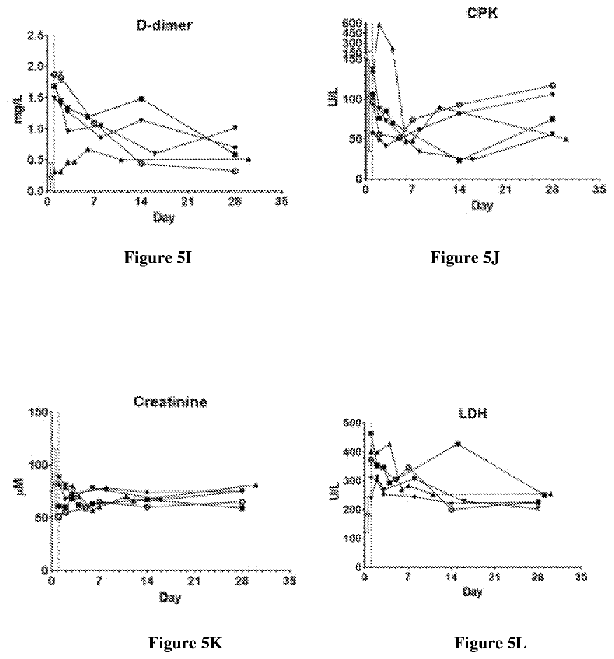

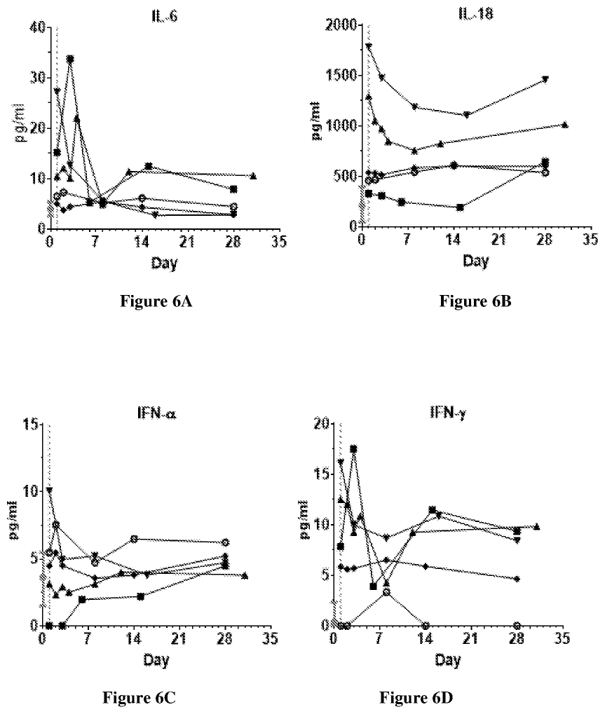

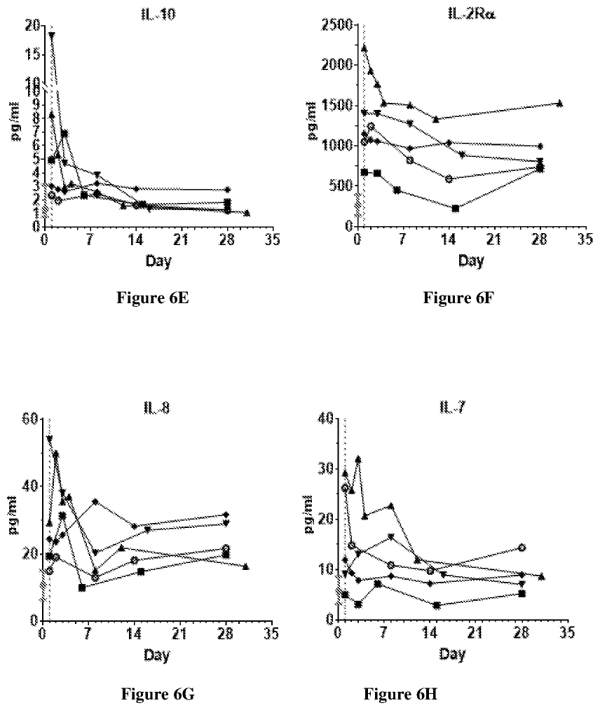

The lab results for each patient are shown in FIGS. 5A-5L and 6A-6H. FIGS. 5A-5L show the Phase I COVID-19 positive biomarkers' profile over time (per day). Markers included WBC (FIG. 5A), Neutrophil % (FIG. 5B), Neutrophil Count (FIG. 5C), Lymphocyte % (FIG. 5D), Lymphocyte count (FIG. 5E), Platelet Count (FIG. 5F), CRP (FIG. 5G), Ferritin (FIG. 5H), D-dimer (FIG. 5I), CPK (FIG. 5J), Creatinine (FIG. 5K), and LDH (FIG. 5L). FIGS. 6A-6H show Phase I COVID-19 positive cytokine profile over time (per day). Cytokines measured included IL-6 (FIG. 6A), IL-18 (FIG. 6B), IFN-α (FIG. 6C), IFN-γ (FIG. 6D), IL-10 (FIG. 6E), IL-2Ra (FIG. 6F), IL-8 (FIG. 6G), and IL-7 (FIG. 6H).

Lymphocyte count improved (FIG. 5E), and levels of CRP (FIG. 5G), ferritin (FIG. 5H), and D-dimer (FIG. 5I) decreased following treatment with Allocetra-OTS, correlating with amelioration of the clinical status. All patients had mild elevation of liver enzymes before Allocetra administration that resolved by day 28. Most notably, all patients were discharged with a negative PCR for SAR-CoV-2 (COVID-19). As shown in FIGS. 6A-6H, the cytokine storm resolved following treatment with Allocetra. Interestingly IFN-α (IFN-alpha) was increased in most patients.

Interim Summary Results for Phase II Trial.

Twenty-one severe and critical patients (non-ventilated) were recruited to-date so far (5 in Phase Ib, 16 in Phase II). Eighteen patients have completed the 28-day assessment. Current patient characteristics were as follows: Males/females (16/4), Average age 57 (37-81), Obesity (9/20), hypertension (7/20). Fourteen of the 16 patients who participated in the Phase II trial have been released to their home within an average time of 5.3 days from the first administration of the Allocetra therapy.

The Phase II study is still on going. Following single dose administration of Allocetra, in combination with SOC treatment, (1) 11/11 of the severe patients were discharged healthy, with an average duration of hospitalization post Allocetra treatment of 5.3 days; (2) 7/9 of the critical patients were discharged healthy, having an average duration of hospitalization post Allocetra treatment of 7.6 days; and (3) 2/9 of the critical patients were ventilated in ICU on day 28. Table 15 presents patient characteristics and outcomes.

TABLE 15
Patient Characteristics and Outcomes

| | | | | | Avg. days |
|---|---|---|---|---|---|
| | | Discharged | In ICU | Mortality | to |
| | # Patients | healthy | Day-28 | Day-28 | discharge |
| Severe | | | | | |
| No ARDS | 1 | 1/1 (100%) | 0/1 (0%) | 0/1 (0%) | |
| Mild | 10 | 10/10 (100% | 0/10 (0%) | 0/10 (0%) | |
| ARDS | | | | | |
| Total | 11 | 11/11 (100%) | 0/11 (0%) | 0/11 (0%) | 5.3 |
| severe | | | | | |
| Critical | | | | | |
| Moderate | 6 | 6/6 (100%) | 0/6 (0%) | 0/6 (0%) | |
| ARDS | | | | | |
| Severe | 3 | 1/3 (33%) | 2/3 (67%) | 0/3 (0%) | |
| ARDS | | | | | |
| Total | 9 | 7/9 (77%) | 2/9 (23%) | 0/8 (0%) | 7.6 for |
| critical | | | | | discharged |
| Total for | 20 | 18/20 (90%) | 2/20 (10%) | 0/20 (0%) | 6.07 for |
| all 20 | | | | | discharged |

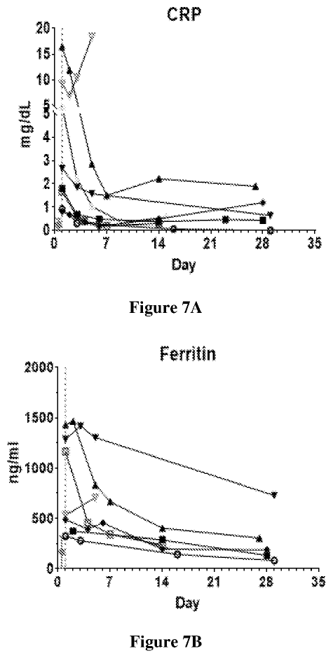

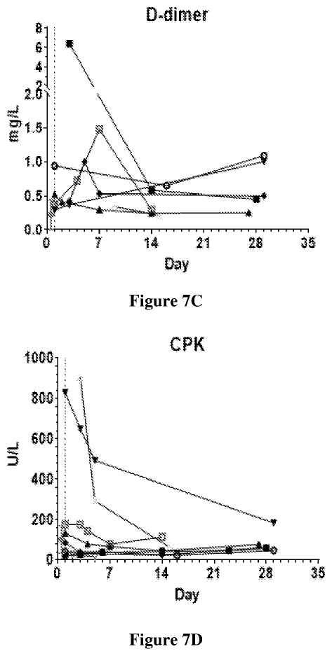

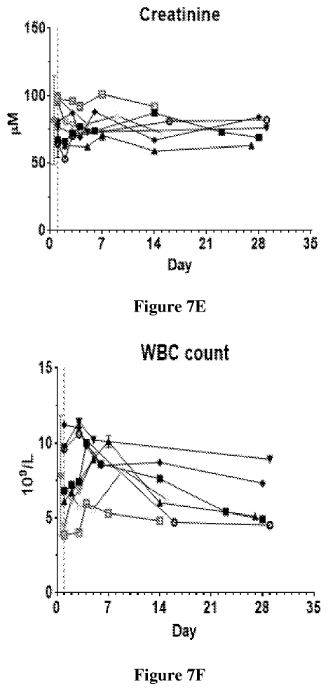

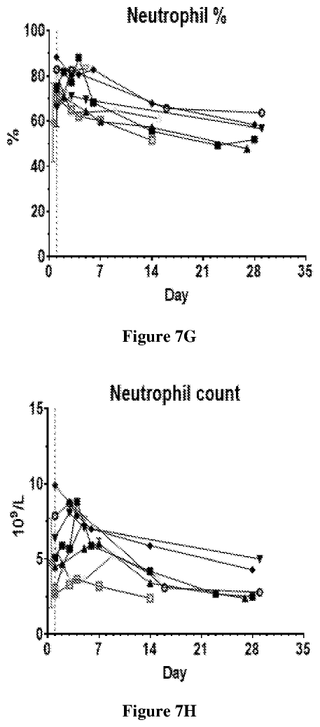

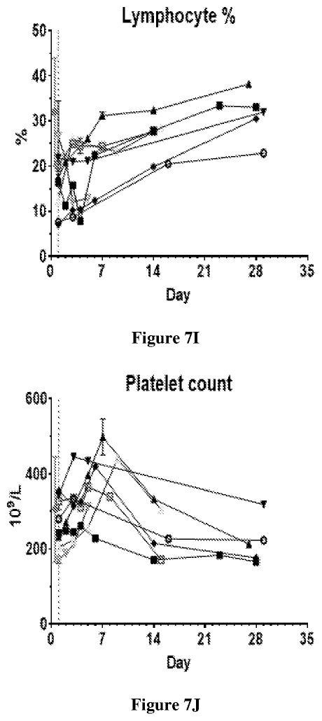

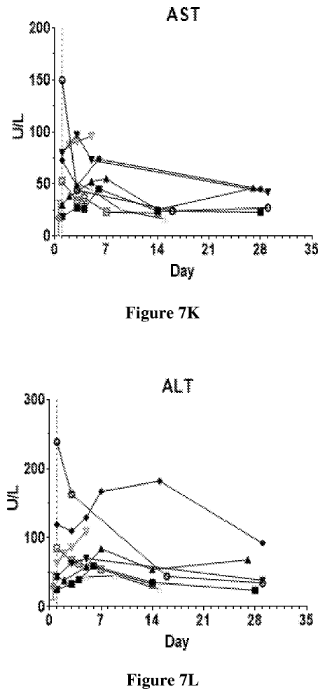

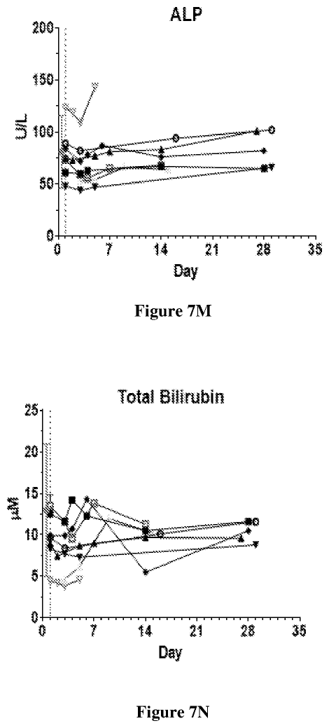

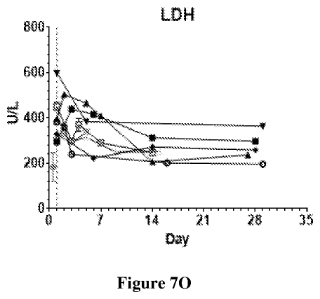

FIGS. 7A-7O show the interim measurements of Phase II COVID-19 positive biomarkers' profile over time (per day). Markers included CRP (FIG. 7A), Ferritin (FIG. 7B), D-dimer (FIG. 7C), CPK (FIG. 7D), Creatinine (FIG. 7E), WBC (FIG. 7F), Neutrophil % (FIG. 7G), Neutrophil Count (FIG. 7H), Lymphocyte % (FIG. 7I), Lymphocyte Count (FIG. 7J), Aspartate transaminase (AST) (FIG. 7K), Alanine aminotransferase (ALT) (FIG. 7L), Alkaline phosphatase (ALP) (FIG. 7M), Total Bilirubin (FIG. 7N), and Lactate dehydrogenase (LDH) (FIG. 7O).

Safety: Four serious adverse effects (SAEs; not related to Allocetra) were documented in 20 of the patients so far: 2 patients progressed to a ventilator followed by extracorporeal membrane oxygenation (ECMO). One AE was possibly related to the Allocetra administration. That reported AE was short shivering towards the end of Allocetra administration in patient 006, which was resolved following IV administration of 12.5 mg of promethazine. This AE was possibly related to the Allocetra administration, but could also have been due to bacteremia, or was possibly a manifestation of COVID-19.

While certain features disclosed herein have been illustrated and described herein, many modifications, substitutions, changes, and equivalents will now occur to those of ordinary skill in the art. It is, therefore, to be understood that the appended claims are intended to cover all such modifications and changes as fall within the true spirit disclosed herein.

1. A method of treating COVID-19 in a subject infected by SARS-CoV-2 virus, said method comprising administering a composition comprising an early apoptotic mononuclear-cell-enriched cell population to the subject, wherein said administration treats COVID-19.

2. The method of claim 1, wherein said treating comprises treating, inhibiting, reducing the incidence of, ameliorating, or alleviating a symptom of COVID-19.

3. The method of claim 2, wherein said symptom comprises organ failure, organ dysfunction, organ damage, a cytokine storm, a cytokine release syndrome, or a combination thereof.

4. The method of claim 3, wherein said organ comprises a lung, a heart, a kidney, or a liver, or any combination thereof.

5. The method of claim 4, wherein said organ dysfunction, failure, or damage comprises lung dysfunction, failure, or damage.

6. The method of claim 5, wherein said lung dysfunction comprises acute respiratory distress syndrome (ARDS) or pneumonia.

7. The method of claim 3, wherein said organ failure comprises acute multiple organ failure.

8. The method of claim 3, wherein said treating organ failure comprises reducing, slowing, inhibiting, reversing, or repairing said organ failure, or a combination thereof.

9. The method of claim 1, wherein said treating increases survival time of a COVID-19 subject, compared with a COVID-19 subject not administered said early apoptotic mononuclear-cell-enriched population.

10. The method of claim 1, wherein said COVID-19 comprises mild, moderate, severe, or critical COVID-19.

11. (canceled)

12. The method of claim 1, wherein the early apoptotic mononuclear-cell-enriched cell population comprises (a) a decreased number of non-quiescent non-apoptotic cells, a suppressed cellular activation of any living non-apoptotic cells, or a reduced proliferation of any living non-apoptotic cells, or (b) a pooled population of early apoptotic mononuclear-enriched cells, or (c) any combination thereof.

13. The method of claim 1, wherein administering comprises a single infusion or multiple infusions of the early apoptotic mononuclear-cell-enriched population.

14. (canceled)

15. The method of claim 1, wherein administering comprises intravenous (IV) administration.

16. The method of claim 1, wherein said early apoptotic mononuclear-cell-enriched population comprises early apoptotic cells irradiated after induction of apoptosis.

17. The method of claim 1, further comprising a step of administering an additional therapy, wherein the additional therapy is administered prior to, concurrent with, or following the step of administering the early apoptotic mononuclear-cell-enriched population.

18. (canceled)

19. The method of claim 1, wherein the method comprises rebalancing the immune response of the subject.

20. The method of claim 19, wherein rebalancing comprises (a) reducing the secretion of one or more proinflammatory cytokines, anti-inflammatory cytokines, chemokine, or immune modulator, or a combination thereof; or wherein rebalancing comprises increasing the secretion of one or more anti-inflammatory cytokine or chemokine, or combination thereof; or a combination thereof.

21. (canceled)

22. (canceled)

23. The method of claim 1, wherein the method reduces the subjects stay in an intensive care unit (ICU), compared with a subject not administered early apoptotic mononuclear-enriched cells.

24. The method of claim 1, wherein the method reduces hospitalization time for said subject, compared with a subject not administered early apoptotic mononuclear-enriched cells.
